SEQUENCE LISTING (116 sequences)

SEQ ID NO: 1 - protein, 699 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKASGGGGGQAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 2 - protein, 588 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 3 - protein, 717 aa, synthetic construct
SPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEPASGGGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGSQVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 4 - protein, 713 aa, synthetic construct
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLAGGGGGPAGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGSEVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGGGGSGGGGSGGGGSQAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVL

SEQ ID NO: 5 - protein, 701 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKASGGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 616 aa, synthetic construct
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 7 - protein, 603 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 8 - protein, 629 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 9 - protein, 576 aa, synthetic construct
QAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 10 - protein, 716 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSHLEQPQISSTKTLSKTARLECVVSGITISATSVYWYRERPGEVIQFLVSISYDGTVRKESGIPSGKFEVDRIPETSTSTLTIHNVEKQDIATYYCALWDRYYKKLFGSGTTLVVTDGGGGSGGGGSGGGGSQKVTQAQSSVSMPVRKAVTLNCLYETSWWSYYIFWYKQLPSKEMIFLIRQGSDEQNAKSGRYSVNFKKAAKSVALTISALQLEDSAKYFCALGVRAFLRDWGIRVLIFGKGTRVTVEP

SEQ ID NO: 11 - protein, 574 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 12 - protein, 548 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 13 - protein, 216 aa, synthetic construct
QAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 460 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSNPSSTVAVGCLAQDFLPDSITFSWKYKNNSDISSTRGFPSVLRGGKYAATSQVLLPSKDVAQGTNEHVVCKVQHPNGNKEKDVPLPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 603 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 16 - protein, 228 aa, synthetic construct
HLEQPQISSTKTLSKTARLECVVSGITISATSVYWYRERPGEVIQFLVSISYDGTVRKESGIPSGKFEVDRIPETSTSTLTIHNVEKQDIATYYCALWDRYYKKLFGSGTTLVVTDGGGGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 459 aa, synthetic construct
QKVTQAQSSVSMPVRKAVTLNCLYETSWWSYYIFWYKQLPSKEMIFLIRQGSDEQNAKSGRYSVNFKKAAKSVALTISALQLEDSAKYFCALGVRAFLRDWGIRVLIFGKGTRVTVEPGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 609 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGGGGSGGGGSGGGGSQAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 19 - protein, 241 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGEC

SEQ ID NO: 20 - protein, 588 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLA

SEQ ID NO: 21 - protein, 469 aa, synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 22 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGEC

SEQ ID NO: 23 - protein, 457 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 469 aa, synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAGGGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 25 - protein, 586 aa, synthetic construct
QKVTQAQSSVSMPVRKAVTLNCLYETSWWSYYIFWYKQLPSKEMIFLIRQGSDEQNAKSGRYSVNFKKAAKSVALTISALQLEDSAKYFCALGVRAFLRDWGIRVLIFGKGTRVTVEPGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 26 - protein, 577 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGGSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKP

SEQ ID NO: 27 - protein, 600 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGPAGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 28 - protein, 725 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGPAGGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGGGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 29 - protein, 562 aa, synthetic construct
QAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLGGGGGGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEP

SEQ ID NO: 30 - protein, 699 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGGGGGGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGQVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 31 - protein, 501 aa, synthetic construct
QITCPPPMSVEHADIWVKSYSLYSRERYICNSGFKRKAGTSSLTECVLNKATNVAHWTTPSLKCIRDPALGGGGGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGAPGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 32 - protein, 702 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINGGGGGGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGAPGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGQVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 33 - protein, 512 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRGRAGSGATNFSLLKQAGDVEENPGPMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAP

SEQ ID NO: 34 - protein, 454 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 558 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRGRAGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 36 - protein, 630 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAP

SEQ ID NO: 37 - protein, 699 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRGRAGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 38 - protein, 448 aa, synthetic construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 497 aa, synthetic construct
HLEQPQISSTKTLSKTARLECVVSGITISATSVYWYRERPGEVIQFLVSISYDGTVRKESGIPSGKFEVDRIPETSTSTLTIHNVEKQDIATYYCALWDRYYKKLFGSGTTLVVTDGGGGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGPAGPTTTPAPRPPTPAPTIASQPLSLRPEACRPAAGGAVHTRGLDFACDIYIWAPLAGTCGVLLLSLVITLYCKRGRAGSGATNFSLLKQAGDVEENPGPMRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKK

SEQ ID NO: 40 - protein, 720 aa, synthetic construct
QKVTQAQSSVSMPVRKAVTLNCLYETSWWSYYIFWYKQLPSKEMIFLIRQGSDEQNAKSGRYSVNFKKAAKSVALTISALQLEDSAKYFCALGVRAFLRDWGIRVLIFGKGTRVTVEPGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 41 - protein, 476 aa, synthetic construct
HLEQPQISSTKTLSKTARLECVVSGITISATSVYWYRERPGEVIQFLVSISYDGTVRKESGIPSGKFEVDRIPETSTSTLTIHNVEKQDIATYYCALWDRYYKKLFGSGTTLVVTDGGGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRGRAGSGATNFSLLKQAGDVEENPGPMEFGLSWVFLVALLRGVQCAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 42 - protein, 611 aa, synthetic construct
QKVTQAQSSVSMPVRKAVTLNCLYETSWWSYYIFWYKQLPSKEMIFLIRQGSDEQNAKSGRYSVNFKKAAKSVALTISALQLEDSAKYFCALGVRAFLRDWGIRVLIFGKGTRVTVEPGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKALIHLSDLREYRRFEKEKLKSQWNNDNPLFKSATTTVMNPKFAESGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 43 - protein, 427 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKLGFFKRQYKDMMSEGGPPGAEPQGGSVKFSRSADAPAYQQGQNQLYNELNLGRREEYDVLDKRRGRDPEMGGKPQRRKNPQEGLYNELQKDKAEAYSEIGMKGERRRGKGHDGLYQGLSTATKDTYDALHMQALPPR

SEQ ID NO: 44 - protein, 389 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKALIHLSDLREYRRFEKEKLKSQWNNDNPLFKSATTTVMNPKFAESGGSRGRKKLLYIFKQPFMRPVQTTQEEDGCSCRFPEEEEGGCEL

SEQ ID NO: 45 - protein, 612 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEFGLSWVFLVALLRGVQCAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 46 - protein, 399 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 47 - protein, 397 aa, synthetic construct
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAGGGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 48 - protein, 581 aa, synthetic construct
QVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 49 - protein, 420 aa, synthetic construct
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLAGGGGGHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLAGAPGGGRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGPTTTPAPRPPTPAPTIASQPLSLRPEACRPAAGGAVHTRGLDFACDIYIWAPLAGTCGVLLLSLVITLYCKRR

SEQ ID NO: 50 - protein, 396 aa, synthetic construct
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLAPAGGGGGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLGGPAGPTTTPAPRPPTPAPTIASQPLSLRPEACRPAAGGAVHTRGLDFACDIYIWAPLAGTCGVLLLSLVITLYCKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 51 - protein, 101 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNV

SEQ ID NO: 52 - protein, 100 aa, synthetic construct
GSASAPTLFPLVSCENSSPSSTVAVGCLAQDFLPDSITFSWKYKNNSDISSTRGFPSVLRGGKYAATSQVLLPSKDVMQGTDEHVVCKVQHPNGNKEKDV

SEQ ID NO: 53 - protein, 100 aa, synthetic construct
GSASAPTLFPLVSCENSNPSSTVAVGCLAQDFLPDSITFSWKYKNNSDISSTRGFPSVLRGGKYAATSQVLLPSKDVAQGTNEHVVCKVQHPNGNKEKDV

SEQ ID NO: 54 - protein, 96 aa, synthetic construct
GSASAPTLFPLVSCDTSSVAVGCLAQDFLPDSITFSWKYKNNSDISSTRGFPSVLRGGKYAATSQVLLPSKDVMQGTDEHVVCKVQHPNGNKEKNV

SEQ ID NO: 55 - DNA, 130 bp, unidentified
  source 1..130: mol_type = other DNA
ctttcttgctgtccaatttctattaaaggttcctttgttccgtaagtccaactactaaactgggggatattatgaagggccttgagcatctggattctgcctaataaaaaacatttattttcattgcaat

SEQ ID NO: 56 - protein, 606 aa, synthetic construct
DIQLTQSPSSLSASVGDRVSITCKASQDVSIAVAWYQQKPGKAPKLLIYSASYRTTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 57 - protein, 459 aa, synthetic construct
QVQLGQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 617 aa, synthetic construct
QVQLGQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 59 - protein, 699 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGQVQLGQSGSELKKPGASVKVSCKASGYTFTSYAMNWVRQAPGQGLKWMGWINTYTGETPYTDDFKGRFAFSLDTSVSTAYLQISSLKADDTAVYFCARGGFGSSYWYFDVWGQGTLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 60 - protein, 460 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 538 aa, synthetic construct
DIQLTQSPSSLSASVGDRVSITCKASQDVSIAVAWYQQKPGKAPKLLIYSASYRTTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 62 - protein, 716 aa, synthetic construct
QVQLGQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKPAGGGGGSQAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLGGGGSGGGGSGGGGSEVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSS

SEQ ID NO: 63 - protein, 540 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 64 - protein, 550 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 65 - protein, 549 aa, synthetic construct
QVQLGQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 66 - protein, 615 aa, synthetic construct
DIQLTQSPSSLSASVGDRVSITCKASQDVSIAVAWYQQKPGKAPKLLIYSASYRTTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKQKVYACEVTHQGLSSPVTKSFLRGECGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLV

SEQ ID NO: 67 - protein, 599 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGCGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGAPGGGGSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 68 - protein, 609 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGAPGGGGSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 69 - protein, 361 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 70 - protein, 615 aa, synthetic construct
EVQLVESGGGLVQPGGSLKLSCAASGFTFNTYAMNWVRQAPGKGLEWVGRIRSKYNNYATYYADSVKGRFTISRDDSKNTLYLQMNSLKTEDTAVYYCVRHGNFGNSYVSWFAYWGQGTLVTVSSGGGGSGGGGSGGGGSQAVVTQEPSLTVSPGGTVTLTCGSSTGAVTTSNYANWVQQKPGQAFRGLIGGTNKRAPGTPARFSGSLLGGKAALTLSGAQPEDEAEYYCALWYSNLWVFGGGTKLTVLASGGGGGPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVPAGGGGGTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 451 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGAPGGGGGPPCPPCPAPEAAGASSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 72 - protein, 519 aa, synthetic construct
QIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGAPGGGGGPPCPPCPAPEAAGASSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 73 - protein, 476 aa, synthetic construct
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPAGGGGSGGPPCPPCPAPEAAGASSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 74 - protein, 817 aa, synthetic construct
QRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGAPGGGGGPPCPPCPAPEAAGASSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 75 - protein, 214 aa, synthetic construct
EIVLTQSPATLSLSPGERATLSCRASQSIGTSIHWYQQKPGQAPRLLIKYASESISGIPARFSGSGSGTDFTLTISSLEPEDFAVYYCQQSYIWPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 598 aa, synthetic construct
QVQLVQSGAEVKKPGASVKVSCKASGYTFTNYWLGWVRQAPGQGLEWMGNIFPGGDYTNYNENFKGRVTMTRDTSISTAYMELSRLRSDDTAVYYCARSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVESKYGPPCPPCPAPEAAGASSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLPKGGGGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 77 - protein, 778 aa, synthetic construct
QVQLVQSGAEVKKPGASVKVSCKASGYTFTNYWLGWVRQAPGQGLEWMGNIFPGGDYTNYNENFKGRVTMTRDTSISTAYMELSRLRSDDTAVYYCARSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLV

SEQ ID NO: 78 - protein, 778 aa, synthetic construct
QVQLVQSGAEVKKPGASVKVSCKASGYTFTNYWLGWVRQAPGQGLEWMGNIFPGGDYTNYNENFKGRVTMTRDTSISTAYMELSRLRSDDTAVYYCARSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLV

SEQ ID NO: 79 - protein, 760 aa, synthetic construct
QIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGAPAGGGGGGPPCPPCPAPEAAGASSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 80 - protein, 388 aa, synthetic construct
DIQLTQSPSSLSASVGDRVSITCKASQDVSIAVAWYQQKPGKAPKLLIYSASYRTTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGTHTCPPCPAPEAAGGPGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 81 - protein, 393 aa, synthetic construct
QVQLGQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGTHTCPPCPAPEAAGGPGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 82 - protein, 281 aa, synthetic construct
EIVLTQSPATLSLSPGERATLSCRASQSIGTSIHWYQQKPGQAPRLLIKYASESISGIPARFSGSGSGTDFTLTISSLEPEDFAVYYCQQSYIWPTTFGQGTKLEIKGGGTHTCPPCPAPEAAGGPGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 83 - protein, 287 aa, synthetic construct
QVQLVQSGAEVKKPGASVKVSCKASGYTFTNYWLGWVRQAPGQGLEWMGNIFPGGDYTNYNENFKGRVTMTRDTSISTAYMELSRLRSDDTAVYYCARSMDYWGQGTLVTVSSGGGTHTCPPCPAPEAAGGPGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 84 - protein, 554 aa, synthetic construct
QVQLVQSGAEVKKPGASVKVSCKASGYTFTNYWLGWVRQAPGQGLEWMGNIFPGGDYTNYNENFKGRVTMTRDTSISTAYMELSRLRSDDTAVYYCARSMDYWGQGTLVTVSSGGGGSGGGGSGGGGSEIVLTQSPATLSLSPGERATLSCRASQSIGTSIHWYQQKPGQAPRLLIKYASESISGIPARFSGSGSGTDFTLTISSLEPEDFAVYYCQQSYIWPTTFGQGTKLEIKGGGGASGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGGGSGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 85 - protein, 644 aa, synthetic construct
QVQLVQSGAEVKKPGASVKVSCKASGYTFTNYWLGWVRQAPGQGLEWMGNIFPGGDYTNYNENFKGRVTMTRDTSISTAYMELSRLRSDDTAVYYCARSMDYWGQGTLVTVSSGGGGSGGGGSGGGGSEIVLTQSPATLSLSPGERATLSCRASQSIGTSIHWYQQKPGQAPRLLIKYASESISGIPARFSGSGSGTDFTLTISSLEPEDFAVYYCQQSYIWPTTFGQGTKLEIKGGGGASGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGGGSGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 86 - protein, 428 aa, synthetic construct
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQGGGGGSGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGGGGSGGGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 87 - protein, 677 aa, synthetic construct
QRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGGASCKVPVKKATVVYQGERVKIQEKFKNGMLHGDKVSFFCKNKEKKCSYTEDAQCIDGTIEVPKCFKEHSSLAFWKTDASDVKPCGGGGGGASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 88 - protein, 21 aa, synthetic construct
RGECGGGAPGGGDKTHTCPPC

SEQ ID NO: 89 - protein, 21 aa, synthetic construct
DVPLGGGAPGGGDKTHTCPPC

SEQ ID NO: 90 - protein, 20 aa, synthetic construct
RGECGGGAPGGDKTHTCPPC

SEQ ID NO: 91 - protein, 20 aa, synthetic construct
DVPLGGGAPGGDKTHTCPPC

SEQ ID NO: 92 - protein, 17 aa, synthetic construct
RGECGGGGDKTHTCPPC

SEQ ID NO: 93 - protein, 17 aa, synthetic construct
DVPLGGGGDKTHTCPPC

SEQ ID NO: 94 - protein, 15 aa, synthetic construct
RGESGGDKTHTCPPC

SEQ ID NO: 95 - protein, 15 aa, synthetic construct
DVPLGGDKTHTCPPC

SEQ ID NO: 96 - protein, 15 aa, synthetic construct
RGECGGDKTHTCPPC

SEQ ID NO: 97 - protein, 15 aa, synthetic construct
DVPLGGDKTHTCPPC

SEQ ID NO: 98 - protein, 19 aa, synthetic construct
RGECGGGAPGGGGGTCPPC

SEQ ID NO: 99 - protein, 19 aa, synthetic construct
DVPLGGGAPGGGGGTCPPC

SEQ ID NO: 100 - protein, 19 aa, synthetic construct
RGECPAGGGGGGTHTCPPC

SEQ ID NO: 101 - protein, 19 aa, synthetic construct
DVPLPAGGGGGGTHTCPPC

SEQ ID NO: 102 - protein, 15 aa, synthetic construct
RGECGGGGTHTCPPC

SEQ ID NO: 103 - protein, 15 aa, synthetic construct
DVPLGGGGTHTCPPC

SEQ ID NO: 104 - protein, 13 aa, synthetic construct
RGESGGTHTCPPC

SEQ ID NO: 105 - protein, 13 aa, synthetic construct
DVPLGGTHTCPPC

SEQ ID NO: 106 - protein, 13 aa, synthetic construct
RGECGGTHTCPPC

SEQ ID NO: 107 - protein, 13 aa, synthetic construct
DVPLGGTHTCPPC

SEQ ID NO: 108 - protein, 19 aa, synthetic construct
RGECGGPAGGGGGTHTCPP

SEQ ID NO: 109 - protein, 18 aa, synthetic construct
NVPLPAGGGGGTHTCPPC

SEQ ID NO: 110 - protein, 20 aa, synthetic construct
RGECGGPAGGGGGTHTCPPC

SEQ ID NO: 111 - protein, 21 aa, synthetic construct
DVPLPGGPAGGGGGTHTCPPC

SEQ ID NO: 112 - DNA, 130 bp, unidentified; identical to SEQ ID NO: 55
  source 1..130: mol_type = other DNA

SEQ ID NO: 113 - DNA, 144 bp, unidentified
  source 1..144: mol_type = other DNA
ttccagccagacacccgccccccggccctggctaagaagttgcttcctgttgccagcatgacctaccctcgcctctttgatgccatccgctgccacctccttttgctcctggaccctttagcctctctgcccttccactctctg

SEQ ID NO: 114 - DNA, 136 bp, unidentified
  source 1..136: mol_type = other DNA
ctggtactgcatgcacgcaatgctagctgcccctttcccgtcctgggcaccccgagtctcccccgacctcgggtcccaggtatgctcccacctccacctgccccactcaccacctctgctagttccagacacctcc

SEQ ID NO: 115 - DNA, 139 bp, unidentified
  source 1..139: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccac

SEQ ID NO: 116 - DNA, 155 bp, unidentified
  source 1..155: mol_type = other DNA
gccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccc

(19) United States
(12) Patent Application Publication
(10) Pub. No.: US 2024/0400705 A1
(43) Pub. Date: Dec. 5, 2024

(54) COMPOSITION OF RECOMBINANT ANTIGEN BINDING MOLECULES AND METHOD OF MAKING AND USING THEREOF

(71) Applicant: Synergy IMT, Inc., Kirkland, WA (US)

(72) Inventors: DONALD E. STAUNTON, Kirkland, WA (US); AMA SIKORSKI, Kirkland, WA (US); JOHN M HARLAN, Kirkland, WA (US)

(73) Assignee: Synergy IMT, Inc., Kirkland, WA (US)

(21) Appl. No.: 18/694,342

(22) PCT Filed: Sep. 26, 2022
(86) PCT No.: PCT/US2022/077041

§ 371 Date: Mar. 21, 2024

Related U.S. Application Data
(60) Provisional application No. 63/248,322, filed on Sep. 24, 2021.

(51) Int. Cl. C07K 16/28; C07K 16/30

(52) U.S. Cl. CPC C07K 16/2878; C07K 16/2809; C07K 16/30; C07K 2317/31; C07K 2317/622

(57) ABSTRACT

The present invention is related to multispecific recombinant antigen binding molecules characterized by heterodimerization of a first subunit consisting of an Ig heavy chain, a second subunit consisting of a light chain fused to a hinge-CH2-CH3 domain, and at least a first scFv or other type of ligand binding domain that is linked through a first linker to either the N-terminus or the C-terminus of the multispecific recombinant antigen binding molecule, wherein the first subunit and the second subunit are correctly paired through a CH1-CL interaction and stabilized through inter-chain disulfide bonds within a modified hinge region.

### CROSS-REFERENCE TO RELATED APPLICATIONS

This U.S. Non-provisional patent application, which was filed as U.S. patent application Ser. No. 18/694,342 on Mar. 21, 2024, claims priority under 35 U.S.C. § 371 to PCT Patent Application No. PCT/US2022/077041, which was filed on Sep. 26, 2022 and claims the benefit of U.S. Provisional Patent Application No. 63/248,322, which was filed on Sep. 24, 2021. The entirety of PCT Patent Application No. PCT/US2022/077041 and U.S. Provisional Patent Application No. 63/248,322 is incorporated herein by reference.

### STATEMENT REGARDING SEQUENCE LISTING

The Sequence Listing XML associated with this application is provided in XML format and is hereby incorporated by reference into the specification. The name of the XML file containing the sequence listing is 4768-P1WO_Seq_List_20220923_ST26.xml. The XML file is 149 KB; was created on Sep. 23, 2022; and is being submitted via Patent Center with the filing of the specification.

### BACKGROUND

The present disclosure generally relates to the technical field of cancer immunotherapy, and more particularly to recombinant antigen binding molecules, compositions thereof, and their uses, wherein the molecules have multiple antigen binding specificities and multiple anti-tumor mechanisms.

Unless otherwise indicated herein, the materials described in this section are not prior art to the claims in this application and are not admitted as prior art by inclusion in this section.

Despite the recent advances in drug discovery and clinical imaging, cancer remains one of the deadliest diseases in humans. Our understanding of tumors, particularly how a tumor initiates, survives under stress, colonizes/metastasizes to a distant organ and/or site, and becomes resistant to chemotherapeutic drugs are still limited. The American Cancer Society estimated new cases of cancer in the U.S. in 2014 of 1.6 million, with no approved curative treatment for most of the predominant types of cancer. For example, gastrointestinal (GI) cancers (colorectal, gastric, pancreatic, esophageal, bile duct, and liver) are leading causes of morbidity and mortality worldwide. Colorectal carcinoma (CRC) alone represents approximately 10% of all cancer diagnosis and is the second leading cause of cancer deaths world-wide. In the U.S., colorectal cancer is the third most common cancer (excluding skin cancers) with 104,610 colon and 43,340 rectal cancer cases estimated for 2020.

Harnessing the immune system's anti-tumor mechanisms through immunotherapeutics has shown great promise; however, advances are still urgently needed to effectively treat solid tumors. Bispecific antibodies that target CD3 positive T cells and CD19 positive B cells have proven effective for treating hematologic malignancies (Labrijn A F, Janmaat M L, Reichert J M, Parren PWHI. Bispecific antibodies: a mechanistic review of the pipeline. Nat Rev Drug Discov. 2019 August; 18(8):585-608. doi: 10.1038/s41573-019-0028-1. PMID: 31175342; Yu S, Li A, Liu Q, Yuan X, Xu H, Jiao D, Pestell R G, Han X, Wu K. Recent advances of bispecific antibodies in solid tumors. J Hematol Oncol. 2017 Sep. 20; 10(1):155. doi: 10.1186/s13045-017-0522-z. PMID: 28931402; PMCID: PMC5607507; Suurs F V, Lub-de Hooge M N, de Vries E G E, de Groot D J A. A review of bispecific antibodies and antibody constructs in oncology and clinical challenges. Pharmacol Ther. 2019 September; 201:103-119. doi: 10.1016/j.pharmthera.2019.04.006. Epub 2019 Apr. 24. PMID: 31028837; Bates A, Power C A. David vs. Goliath: The Structure, Function, and Clinical Prospects of Antibody Fragments. Antibodies (Basel). 2019 Apr. 9; 8(2):28. doi: 10.3390/antib8020028. PMID: 31544834; PMCID: PMC6640713). Attempts to effectively treat solid tumors with bispecific antibodies, however, has had limited success, possibly due to unfavourable biodistribution and insufficient anti-tumor activity. Lack of efficacy can be due to dose-limiting toxicities or loss of tumor target antigen expression. Improved immunotherapies combined with improved multispecific antibody-based recombinant antigen binding molecules are needed to more effectively: 1) stimulate more than one mechanism of anti-tumor activity; and 2) enhance biodistribution for the efficient and safe localization of anti-tumor activity. Therefore, there is a need for an improved multispecific antibody-based scaffold with improved target antigen binding affinities to better support biodistribution and stimulation of multiple mechanisms of anti-tumor activity and there is further need for such multispecific, multifunctional, scaffolds that can be incorporated into chimeric antigen receptor (CAR) cell therapies.

### SUMMARY

This summary is provided to introduce a selection of concepts in a simplified form that is further described below in the Detailed Description. This summary is not intended to identify key features of the claimed subject matter, nor is it intended to be used as an aid in determining the scope of the claimed subject matter.

Disclosed herein are embodiments of an antibody-derived recombinant antigen binding molecule.

In one aspect, the antibody-derived recombinant antigen binding molecule can comprise a scaffold with at least a first subunit having an N-terminus and a C-terminus, at least a second subunit having an N-terminus and a C-terminus, at least a third subunit having an N-terminus and a C-terminus, and at least a fourth subunit having an N-terminus and a C-terminus. The first subunit can comprise from the N-terminus to the C-terminus, a first antigen binding domain (A) and a CL light chain constant region of an Igκ or an Igλ. The second subunit can comprise from the N-terminus to the C-terminus, a second antigen binding domain (B), a CH1 heavy chain constant region of an IgA, an IgM, an IgG₂, or an IgG₃ (Ig-M/A/G₂/G₃), an immunoglobulin hinge region, a heavy chain Fc domain from an IgG, and a third antigen binding domain (C). The third subunit can comprise from the N-terminus to the C-terminus, a fourth antigen binding domain (D), a CL light chain constant region of an Igκ or an Igλ, an immunoglobulin hinge region, a heavy chain Fc domain from an IgG, and a fifth antigen binding domain (E). The fourth subunit can comprise from the N-terminus to the C-terminus, a sixth antigen binding domain (F) and a CH1 heavy chain constant region of an Ig-M/A/G₂/G₃. The high-affinity binding between the CL light chain constant region in the first subunit and the CH1 heavy chain constant region in the second subunit and a second high-affinity binding between the CL light chain constant region in the third subunit and the CH1 heavy chain constant region in the fourth subunit can drive heterodimerization of the molecule.

In another aspect, the antibody-derived recombinant antigen binding molecule can comprise at least a first subunit having an N-terminus and a C-terminus and at least a second subunit having an N-terminus and a C-terminus. The first subunit can comprise from the N-terminus to the C-terminus, a first antigen binding domain (A) and a CH1 heavy chain constant region of an Ig-M/A/G₂/G₃. The second subunit can comprise from the N-terminus to the C-terminus, a second antigen binding domain (B) and a CL light chain constant region of an Igκ or an Igλ. The first subunit and the second subunit are linked to a chimeric antigen receptor, wherein the chimeric antigen receptor can comprise a hinge region, a transmembrane domain, and at least one intracellular signaling domain. The high-affinity binding between the CH1 heavy chain constant region in the first subunit and the CL light chain constant region in the second subunit can drive heterodimerization of the molecule. In some embodiments, the chimeric antigen receptor is a phagocytic chimeric antigen receptor.

In another aspect of the invention, the disclosure provides for a single chain antibody-derived recombinant antigen binding molecule. The single chain antibody-derived recombinant antigen binding molecule can comprise an antigen binding domain and at least one subunit of a tumor necrosis factor superfamily (TNFSF) ligand, wherein at least one subunit of the TNFSF ligand is tethered by a flexible linker to an N-terminal and/or a C-terminal antigen binding domain.

In another aspect of the invention, the disclosure provides for an antibody-derived recombinant antigen binding molecule comprising a scaffold with at least a first subunit having an N-terminus and a C-terminus, at least a second subunit having an N-terminus and a C-terminus; and at least one subunit of a tumor necrosis factor superfamily (TNFSF) ligand; the first subunit, comprising from the N-terminus to the C-terminus, a first antigen binding domain, a CH1 heavy chain constant region, an immunoglobulin hinge region, a heavy chain Fc domain from an IgG, and a second antigen binding domain; and the second subunit, comprising from the N-terminus to the C-terminus, a third antigen binding domain, a CL light chain constant region of an Igκ or an Igλ, an immunoglobulin hinge region, a heavy chain Fc domain from an IgG, and a fourth antigen binding domain; and wherein the TNFSF ligand is linked to a terminal end of the first and/or second subunit.

In another aspect of the invention, the disclosure provides for a genetically modified host cell that can produce an antibody-derived recombinant antigen binding molecule as described above, wherein the genetic modification comprises a recombinant expression vector, and wherein the genetically modified host cell produces an increased amount of the antibody-derived recombinant antigen binding molecule compared to host cells which are not genetically modified in the same way.

In another aspect of the invention, the disclosure provides for a method to express an antibody-derived recombinant antigen binding molecule. The method can comprise culturing a genetically modified host cell comprising at least one recombinant expression vector that can express an antibody-derived recombinant antigen binding molecule as described above; wherein culturing the genetically modified host cell under conditions such that high-affinity binding between the CL light chain constant region and the CH1 heavy chain constant region drive heterodimerization of the molecule; and purifying the heterodimeric recombinant antigen binding molecule from the culture media.

The foregoing aspects and many of the attendant advantages of this invention will become more readily appreciated as the same become better understood by reference to the following detailed description, when taken in conjunction with the accompanying drawings.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1B illustrate the heterodimeric core structure of the TetrAx recombinant antigen binding molecule. The TetrAx core heterodimerization is supported by a high-affinity CH1-CL binding interface. The Kd and delta-G calculated from IgM IgA1 and IgG1 crystal structures indicates that the IgM CH1 (MCH1)-CL interface Kd is 350-fold lower than that of IgG1.

FIGS. 2A through 2I illustrate different formats for multispecific recombinant antigen binding molecules targeting TNFSF, and certain other immune and tumor cell antigens. FIGS. 2A, 2B, and 2D illustrate the different configurations for the 2-subunit TetrAx antibody-derived recombinant antigen binding molecule. FIG. 2C illustrates the related multispecific homo- or heterodimer molecule incorporating a TetrAx linker-truncated Ig hinge. FIG. 2E through 2I illustrate formats for small targeted TNFSF recombinant antigen binding molecules. Examples of binding domains that can be linked to the N- and C-terminus of subunits are bracketed.

FIGS. 3A through 3E. 3A and 3B demonstrate the relatively high production of TetrAx recombinant antigen binding molecules comprising an IgM CH1 when compared side-by-side with TetrAx recombinant antigen binding molecules comprising IgA1-CH1 or IgG1-CH1 isotypes, in the same system, prior to any process development. FIGS. 3C through 3E show representative SDS-PAGE gels depicting efficient production and one-step purification of various TetrAx recombinant antigen binding molecules.

FIGS. 4A through 4E illustrate SDS-PAGE and flow cytofluorimetry graphs demonstrating that the N-linked glycosylation motif in the TetrAx IgM-CH1 recombinant antigen binding molecule does not have a major impact on an adjacent binding domain specific for T cell CD3.

FIGS. 5A through 5F illustrate the binding of TetrAx recombinant antigen binding molecules to purified-phosphatidylserine or tumor cell phosphatidylserine. FIGS. 5A to 5E are representative flow cytofluorimetry graphs. FIG. 5F is a representative graph of ELISA results both illustrating the binding of TetrAx recombinant antigen binding molecules possessing a b2GP1 domain to the immune check point tumor antigen, phosphatidylserine (PS).

FIGS. 6A through 6D illustrate flow cytofluorimetry graphs representing the binding of the multispecific recombinant antigen-binding molecules to their target antigens on tumor cells (A-C) and T cells (D).

FIGS. 7A through 7D demonstrate T cell activation properties of the multispecific recombinant molecules including activation-induced cell death (AICD) and tumor target cell dependent induction of CD137 expression.

FIG. 8 compares the cytotoxic activity mediated by TetrAx recombinant antigen binding molecules comprised of different CH1 isotypes, a CD3 binding moiety and bivalent b2GP1 D5 targeting of DLD1 tumor cell phosphatidylserine (PS).

FIGS. 9A and 9B compare the cytotoxic activity mediated by T cell engaging TetrAx recombinant antigen binding molecules that are monovalent or bivalent for DLD1 tumor cell phosphatidylserine (PS). FIGS. 9A and 9B also illustrate the results from repeated assays.

FIG. 10 compares the potent cytotoxic activity of T cell engaging MCH1 TetrAx recombinant antigen binding molecules, mono- or bivalent for phosphatidylserine (PS) when targeting DLD1, AGS or SW480 tumor cells.

FIGS. 11A through 11C illustrate that TetrAx specific for TROP2 and CD3 can mediate potent tumor cytotoxicity. FIG. 11A demonstrates that the cytotoxic activity of the MCH1 TetrAx recombinant antigen binding molecule is enhanced >10,000-fold when specificity for tumor cell PS is added to that of Trop2. FIGS. 11B and 11C demonstrate the cytotoxic activity of MCH1 TetrAx recombinant antigen binding molecules targeting AGS and SW480 tumor cell TROP2. FIG. 11D demonstrates DLD1 cytotoxicity mediated by Trop2, CD3, CD17 trispecific (20v8-18v23).

FIG. 12 demonstrates potent tumor cell cytotoxicity using T cell engaging heterodimeric molecules comprised of a NKG2D homodimeric receptor to target NKG2D ligands.

FIGS. 13A and 13B demonstrate potent TetrAx recombinant antigen binding molecule cytotoxicity in a 3D DLD1 tumor model. At 48 hours, TetrAx recombinant antigen binding molecules that target CD3, Trop2 and PS kill ˜60% of the tumor cells with an EC50 of 21 pM whereas TetrAx recombinant antigen binding molecules that target CD3 and are bivalent for PS kill ˜60% of the tumor cells with an EC50 of 7 pM.

FIGS. 14A and 14B illustrate tumor target antigen-dependent T cell signaling through CD137 induced by TetrAx multispecific molecule specific for TROP2 and possessing one (A) or two (B) CD137L monomers.

FIGS. 15A and 15B illustrate an example of robust T cell CD137 signaling mediated by a multispecific TetrAx molecule that binds CD3 and possesses a single CD137L domain (A) that does not result in significant activation-induced cell death (AICD) (B).

FIGS. 16A and 16B demonstrate that a tumor targeted (TROP2) non-CD3 binding multispecific molecule possessing a CD137L dimer (one monomer on each chain of the heterodimer) can stimulate T cell CD137 expression (A) and tumor cell killing (B). The same TetrAx possessing only one CD137L monomer did not induce substantial CD137 expression or tumor cell cytotoxicity.

FIGS. 17A through 17C illustrate a mechanism of tumor antigen dependent CD137 activation in which TetrAx bound to tumor cells can avidly present CD137L dimers to induce T cell activation.

FIGS. 18A through 18E. FIGS. 18A through 18C illustrate the antibody-derived recombinant chimeric antigen binding (MCAR/MPCAR) molecule formats comprising a TetrAx MCH1 core component. FIGS. 18D and 18E illustrate MCAR expression by protein L binding and function by binding to the target antigen Trop2.

### DETAILED DESCRIPTION

While illustrative embodiments have been illustrated and described, it will be appreciated that various changes can be made therein without departing from the spirit and scope of the invention.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The disclosure provides, among others, isolated antibody-based recombinant binding molecules, methods of making such molecules, bispecific or multispecific antigen binding molecules, antigen binding fragments, recombinant antigen binding molecule-drug conjugates and/or immunoconjugates composed from such molecules, and pharmaceutical compositions containing the antibody-based recombinant antigen binding molecules, bispecific or multispecific antigen binding molecules, recombinant antigen binding molecule-drug conjugates and/or immuno-conjugates. The disclosure also provides methods for making the molecules and compositions, and methods for treating cancer using the molecules and compositions as disclosed herein.

### I. Definitions

The term “antibody-based” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody on which the recombinant antigen binding molecule is based can be monoclonal, polyclonal, chimeric, single chain, bispecific or bi-effective, simianized, human and humanized antibodies as well as active fragments thereof. Examples of active fragments of molecules that bind to known antigens include Fab, F(ab′)₂, scFv, and Fv fragments, including the products of a Fab immunoglobulin expression library and epitope-binding fragments of any of the antibodies and fragments mentioned above. In some embodiments, the antibody-based recombinant binding molecule can include portions of an immunoglobulin molecule and immunologically active portions of immunoglobulin molecules, i.e., molecules that contain a binding site that immunospecifically binds an antigen. The immunoglobulin can be of any type (IgG, IgM, IgD, IgE, IgA, and IgY) or class (IgG₁, IgG₂, IgG₃, IgG₄, IgA₁, and IgA₂) or subclasses of immunoglobulin molecule. In one embodiment, the antibody can be whole antibodies and any antigen-binding fragment derived from the whole antibodies.

A typical antibody refers to heterotetrameric protein comprising, typically, two heavy (H) chains and two light (L) chains. Each heavy chain is comprised of a heavy chain variable domain (abbreviated as VH) and 3 heavy chain constant domains (abbreviated as CH1, CH2, and CH3). Each light chain is comprised of a light chain variable domain (abbreviated as VL) and a light chain constant domain (abbreviated as CL). The VH and VL regions can be further subdivided into domains of hypervariable complementarity determining regions (CDR), and more conserved regions called framework regions (FR).

Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interact with an antigen.

As used herein, the term “hinge region” includes the portion of a heavy chain molecule that joins the CH1 domain to the CH2 domain; the CH1 domain to the CH3 domain; or the CH2 domain to the CH3 domain. This hinge region comprises approximately 25 residues and is flexible, thus allowing the two N-terminal antigen binding regions to move independently. Hinge regions can be subdivided into three distinct domains: upper, middle, and lower hinge domains (Roux K H, Strelets L, Brekke O H, Sandlie I, Michaelsen T E. Comparisons of the ability of human IgG3 hinge mutants, IgM, IgE, and IgA2, to form small immune complexes: a role for flexibility and geometry. J Immunol. 1998 Oct. 15; 161(8):4083-90. PMID: 9780179).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure can be made by the hybridoma method first described by Kohler and Milstein (Köhler, G., Milstein, C. Continuous cultures of fused cells secreting antibody of predefined specificity. Nature 256, 495-497 (1975)), or can be made by recombinant DNA methods (see, e.g., U.S. Pat. No. 4,816,567).

The term “monoclonal antibody can include a “chimeric” antibody (immunoglobulin) in which a portion of the heavy and/or light chain is identical with or homologous to corresponding sequences in an antibody derived from a particular species or belonging to a particular antibody class or subclass, while the remainder of the chain(s) is identical with or homologous to corresponding sequences in an antibody derived from another species or belonging to another antibody class or subclass, as well as fragments of such an antibody, so long as they exhibit the desired biological activity (see e.g., U.S. Pat. No. 4,816,567; and Morrison (Morrison S L, Johnson M J, Herzenberg L A, Oi V T. Chimeric human antibody molecules: mouse antigen-binding domains with human constant region domains. Proc Natl Acad Sci USA. 1984 November; 81(21):6851-5, incorporated herein by incorporation).

A monoclonal antibody can be produced using various methods including mouse hybridoma or phage display (Siegel D L. Recombinant monoclonal antibody technology. Transfus Clin Biol. 2002 January; 9(1):15-22 for a review) or from molecular cloning of an antibody directly from primary B cells (see e.g., Tiller T. Single B cell antibody technologies. N Biotechnol. 2011 September; 28(5):453-7).

The term “antigen- or epitope-binding portion or fragment” refers to fragments of an antibody that are capable of binding to an antigen (for example, TROP2, CD19, CD20, and CD22). These fragments have the antigen-binding function and additional functions of the intact antibody. Examples of binding fragments include but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular the CDR3 regions from either the VL and/or VH domains (for example see Beiboer S H, Reurs A, Roovers R C, Arends J W, Whitelegg N R, Rees A R, Hoogenboom H R. Guided selection of a pan carcinoma specific antibody reveals similar binding characteristics yet structural divergence between the original murine antibody and its human equivalent. J Mol Biol. 2000 Feb. 25; 296(3):833-49). Antibody fragments are produced using conventional methods known to those skilled in the art. The antibody fragments can be screened for utility using the same techniques employed with intact antibodies.

The “antigen or epitope binding domains” used to make the recombinant antigen binding molecules of the present disclosure can be derived from an antibody by any number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-669, Academic Press, 1986. (each incorporated herein in their entirety). Further, the “antigen binding domain” is any structural motif that is capable of associating with and binding to an antigen. As used herein, the term “antigen binding molecule” refers to a secondary antigen binding domain linked to a base antigen binding domain through an immunoglobin linker. Thus, an antigen binding molecule is any structural motif that is capable of associating with and binding to an antigen that is linked to an antigen binding domain through an immunoglobin linker.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. The Fab fragment can contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “light chains” of an antibody (immunoglobulin) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG₁, IgG₂, IgG₃, and IgG₄; IgA₁ and IgA₂. The heavy chain constant domains that correspond to the different classes of immunoglobulins are called alpha (α), delta (δ), epsilon (ε), gamma (γ), and μ, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues can be altered to preserve binding affinity. Methods to obtain “humanized antibodies” are well known to those skilled in the art (see, e.g., Queen et al., Proc. Natl. Acad. Sci. USA, 86:10029-10032 (1989), Hodgson et al., Bio/Technology, 9:421 (1991)).

As used herein, the term “chimeric antigen receptor” (MCAR) can bind to more than one target and/or mediate more than one mechanism of activity and comprises an extracellular antigen binding domain supported by a stalk and a transmembrane domain attached to an intracellular domain. The stalk and/or transmembrane domain can originate from various membrane proteins including CD8, CD28, glycosaminoglycans, integrins and the like. The intracellular domain can originate from various TCR or co-stimulatory receptors such as CD3z, CD137, CD40, ICOS, CD2, DAP10, DAP12 or cytokine receptors including IL15, IL7, IL18, and the like.

As used herein, the term “phagocytic chimeric antigen receptor” (MPCAR) can bind to more than one target antigen and mediate more than one mechanism of action, including phagocytosis and comprises an extracellular antigen binding domain supported by a stalk and a transmembrane domain attached to an intracellular domain. The stalk region can originate from various membrane proteins including CD8, CD28, glycosaminoglycans, and the like. The intracellular domain can originate from various phagocytic receptors such as CR1, CD11b/CD18, CD11c/CD18, aVb3, aVb5, LPR1, MER, AXL and scavenger receptors (Lemke G. How macrophages deal with death. Nat Rev Immunol. 2019 September; 19(9):539-549. doi: 10.1038/s41577-019-0167-y. PMID: 31019284; PMCID: PMC6733267). Additionally, the phagocytic antigen receptor structure can result in separation of integrin transmembrane domains and thus mimic the integrin active state to support phagocytic activity.

The terms “polypeptide”, “peptide”, and “protein”, as used herein, are interchangeable and are defined to mean a biomolecule composed of amino acids linked by a peptide bond.

The term “subunit” as used herein refers to a molecule that can bind or interact with a second molecule, i.e., a first subunit binds to a second subunit to form a dimer.

In some aspects of the present description the regions of the recombinant antigen binding molecule described herein are connected with a short linker peptide of 2 to about 15 amino acids. The linker can be rich in glycine for flexibility, as well as serine or threonine for solubility, and can either connect, for example, the C-terminus of the VH with the N-terminus of the VL, or vice versa, and can also be used to operatively associate other domains and/or regions of the recombinant antigen binding molecule described herein.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having a different antigen binding specificity.

“Recombinant” means the recombinant antigen binding molecule or antibody is generated using recombinant nucleic acid techniques in an exogeneous host cell.

The term “antigen” refers to an entity or fragment thereof which can induce an immune response in an organism, particularly an animal, more particularly a mammal including a human. The term includes immunogens and regions thereof responsible for antigenicity or antigenic determinants.

Also, as used herein, the term “immunogenic” refers to substances which elicit or enhance the production of antibodies, T cells or other reactive immune cells directed against an immunogenic agent and contribute to an immune response in humans or animals. An immune response occurs when an individual produces sufficient antibodies, T cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a disassociation constant (KD) for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure can be caused for instance by addition, deletion, substitution, insertion, or recombination.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “mechanism of anti-tumor activity” (MOA) refers broadly to the immune response for preventing or inhibiting tumors or tumor growth. Recombinant antigen binding molecules can stimulate two or three mechanisms of anti-tumor activity. MOAs include T cell or natural killer cell mediated killing and phagocytosis. MOAs can also include natural killer cells killing and blockade of tumor immunosuppression. MOAs can also include antibody dependent cellular cytotoxicity (ADCC) and activation of apoptosis through death domain receptors. MOAs include regulating tumor associated macrophages (TAMs) or cancer associated fibroblasts (CAFs) functioning in the tumor microenvironment (TME). MOAs can also include decreasing immunosuppression and decreasing extracellular matrix survival signals, and broadly disrupting a pro-tumor stroma/microenvironment.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

As used herein, the term “antibody scaffold” refers to a core structure at least comprising a first subunit comprising from the N-terminal end to the C-terminal end of a CL domain linked to a CH2/CH3 IgG Fc domain with a modified immunoglobin hinge and a second subunit comprising from the N-terminal end to the C-terminal end of an Ig-M/A/G₂/G₃ CH1 domain linked to a CH2/CH3 IgG Fc domain with a modified immunoglobin hinge. In some embodiments the core structure (CHAx) is comprised of an Fc domain that comprises a second CH3 dimer located at one of three positions. The antibody scaffold can include the TetrAx scaffold, which comprises 2, 3, or 4 asymmetric subunits or a 4-subunit symmetric core domain; the CHAx core, which comprises a 2-subunit asymmetric core domain; and the chimeric antigen receptor (MCAR) and the phagocytic antigen receptor (MPCAR), which comprise a 2-subunit core domain.

### II. Overview

To enable immunotherapeutics for more effective cancer treatment, especially solid tumors, a combination therapeutic is paramount that incorporates multiple target specificities and/or mechanisms of action beyond that of typical monospecific or bispecific antibodies. A therapeutic that is essentially a combination treatment such as that described here, is necessary to more effectively treat cancer and to increase the frequency of complete and durable clinical responses. Moreover, the therapeutics described here can decrease manufacturing costs by combining more than one mechanism into a single molecule, circumventing the need to produce separate drugs with a single mechanism of activity.

Prior art tri-specific antibodies, that are based on a whole antibody, can have a greater mass and demonstrate decreased tumor penetration relative to the tri-specific recombinant antigen binding molecules described here. While antibodies with a smaller mass, based on antibody fragments, can have greater tumor penetration, they generally possess poor pharmacokinetic properties, such as FDA-approved bispecific antibody Blincyto® that does not possess an Fc region. Specifically, there is a need for an antibody-based scaffold that enables construction of a “combination” therapeutic with more than one mechanism of action, while also having efficient manufacturing, low antigenicity and pharmacokinetic properties similar to prior art mono- or bispecific antibodies. Small targeted TNFSF ligands are also proposed that are designed to induce an anti-tumor immune response over several doses and not be dependent on a long circulatory half-life for efficacy.

In this context, the following Detailed Description illustrates the various antibody-based recombinant antigen binding molecule formats that can be designed for multiple anti-tumor activity, safety, efficient manufacturing, and stability.

The present disclosure can be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### III. Antibody Platform

Disclosed herein are heterodimeric polypeptides referred to as antibody-derived recombinant antigen binding molecules that are characterized by the following core structure or scaffold: identified herein by the names TetrAx, CHAx, STaT, MCAR, and MPCAR. In some embodiments, the antibody-based recombinant antigen binding molecules constitute a versatile modular platform or scaffold in which binding domains can be readily added at various positions to create bivalent, trivalent, tetravalent, or pentavalent antibodies. Positional optimization of binding domains can be readily accomplished through techniques well known to one of ordinary skill in the art. In some embodiments, the binding domains added to the antibody-scaffold can enable bivalent binding to the same target to enhance binding through increased avidity. In some embodiments, the multispecific antigen binding molecules and their relative affinities can optimize biodistribution. In some embodiments, the multispecific antigen binding molecules allow for more than one mechanism of anti-tumor activity. In some embodiments, the TetrAx and CHAx molecules are designed to target certain combinations of tumor antigens and effector cell receptors to stimulate various anti-tumor activities as shown in Table 1.

TABLE 1
lists specific examples of TetrAx and CHAx antibody-based recombinant
antigen binding molecules.

| | | Structural Features of chains possessing | SEQ |
|---|---|---|---|
| | | either MCH1 or CL (& IgG Fc) | ID |
| Format | Specificity | Monomers (from N- to C-terminus) | NO: |
| 2 subunit | Trop2 × CD3 × | CL: RS7scFv-linker-hSP34Vk, CL, | 1 |
| | PDL1 | linker-hinge, CH2—CH3(hole) | |
| | | CH1: hSP34Vh, MCH1wt, linker-hinge, | 2 |
| | | CH2—CH3(knob)-linker PD1 | |
| 2 subunit | Trop2 × CD3 × | CL: PD1 linker-CL, linker-hinge, | 3 |
| | PDL1 × | CH2—CH3, linker, RS7scFv | |
| | TREM2Ls | CH1: TREM2, linker, MCH1wt, linker- | 4 |
| | | hinge, CH2—CH3, SP34scFv | |
| 2 subunit | Trop2 × IL15 × | CL: hRS7scFv, linker, DR5Vk, CL, | 5 |
| | DR5 × | linker-hinge, CH2—CH3 | |
| | TREM2Ls | CH1: DR5Vh, MCH1wt, linker-hinge, | 6 |
| | | CH2—CH3, linker, CD137L | |
| 2 subunit | NKG2DLs × | CL: NKG2D, linker, CL, linker-hinge, | 7 |
| | CD137L × | CH2—CH3, linker, PD1 | |
| | PDL1 | CH1: NKG2D, linker, MCH1wt, linker- | 8 |
| | | hinge, CH2—CH3, linker CD137L | |
| 2 subunit | MR1 × CD3 × | CL: hSP34Vk, CL, linker-hinge, | 9 |
| | PDL1 | CH2—CH3(hole), PD1 | |
| | | CH1: SP34Vh, MCH1wt, linker-hinge, | 10 |
| | | CH2—CH3(knob), linker, sc VyV8 | |
| 2 subunit | Trop2 × PD1 × | CL: RS7Vk, CL, linker-hinge, CH2— | 11 |
| | CD137 × FcR | CH3(hole), PD1 | |
| | | CH1: RS7Vh, MCH1wt, linker-hinge, | 12 |
| | | CH2—CH3, linker, b2GP1 | |
| 3 subunit | Trop2 × CD3 × | CL: hSP34Vk, CL | 13 |
| | PDL1 | CH1: hSP34Vh, MCH1, linker-hinge, | 14 |
| | | CH2—CH3(hole) | |
| | | Linker: hRS7scFv, linker-hinge, CH2— | 15 |
| | | CH3(knob), PD1 | |
| 3 subunit | MR1 × CD3 × | CL: Vg, CL | 16 |
| | PDL1 | CH1: Vd, MCH1, linker-hinge, CH2— | 17 |
| | | CH3(hole) | |
| | | Linker: hSP34scFv, linker-hinge, | 18 |
| | | CH2—CH3(knob). PD1 | |
| 3 subunit | NKG2DLs × | CL: NKG2D, CL | 19 |
| | PtS/Gal9 × | CH1: NKG2D, MCH1wt, linker-hinge, | 20 |
| | PDL1 × | CH2—CH3(hole), TREM2 | |
| | TREM2Ls | Linker: TIM3-linker-hinge, CH2— | 21 |
| | | CH3, PD1 | |
| 3 subunit | TROP2 × PtS × | CL: RS7Vk, CL | 22 |
| | PD1 × CD16 | CH1: RS7Vh, MCH1wt, linker-hinge, | 23 |
| | | CH2—CH3(hole; LL) | |
| | | Linker: TIM3, linker-hinge, CH2— | 24 |
| | | CH3(knob, LL), PD1 | |
| 4 subunit | TROP2 × | CL: Vg, CL | 16 |
| | PDL1 × CD3 × | CH1: Vd, MCH1wt, linker-hinge, | 25 |
| | TIM3L | CH2—CH3(hole), PD1 | |
| | | CH1(2): SP34Vh, MCH1wt, linker- | 26 |
| | | hinge, CH2—CH3(knob), TIM3 | |
| | | CHL(2): SP34Vk, CL | 13 |
| CHAx | NKG2DL × | A1: NKG2D, linker-hinge, CH2, | 27 |
| Core A | DR5 × PD1 | CH3(hole), linker, CH3(knob), PD1 | |
| | | A2: NKG2D, linker-hinge, CH2, | 28 |
| | | CH3(knob), linker, CH3(hole), linker, | |
| | | DR5scFv | |
| CHAx | TROP2 × | B1: SP34Vk, linker, CH3(knob), linker, | 29 |
| Core B | CD3 × PD1 | CH2, CH3(hole), PD1 | |
| | | B1: SP34Vh, linker, CH3(hole), CH2, | 30 |
| | | CH3(knob), linker, RS7scFv | |
| CHAx | TROP2 × | C1: IL15a, linker, CH3(knob), linker- | 31 |
| Core C | IL15Rc × PtS | hinge(LL), CH2, CH3(hole), linker, | |
| | | b2GP1-D5 | |
| | | C2: IL15, linker, CH3(hole), linker- | 32 |
| | | hinge(LL), CH2, CH3(knob), linker, | |
| | | RS7scFv | |

### A. Structure

### 1. Antigen-Binding Molecules

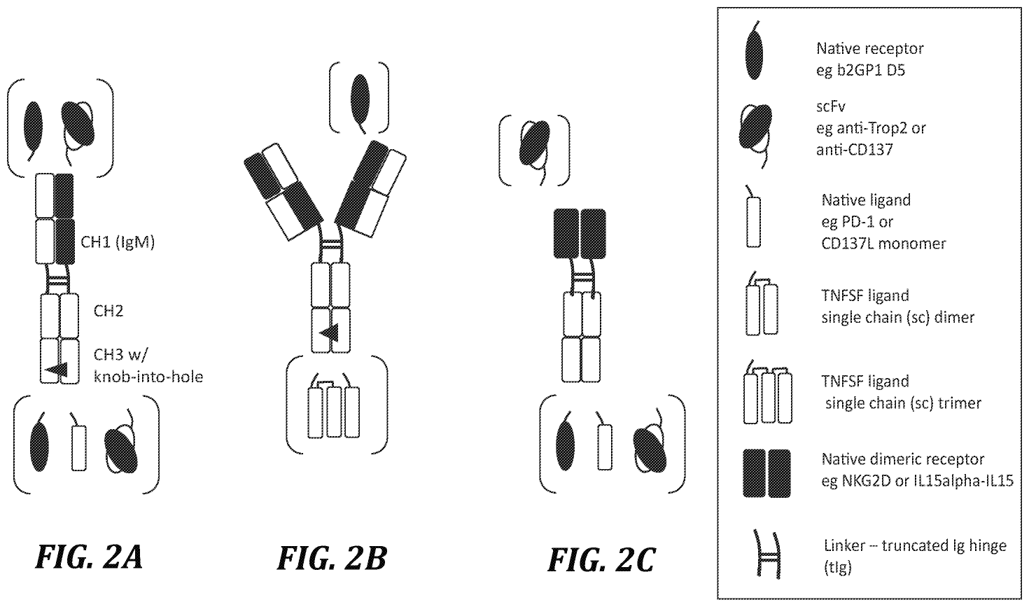

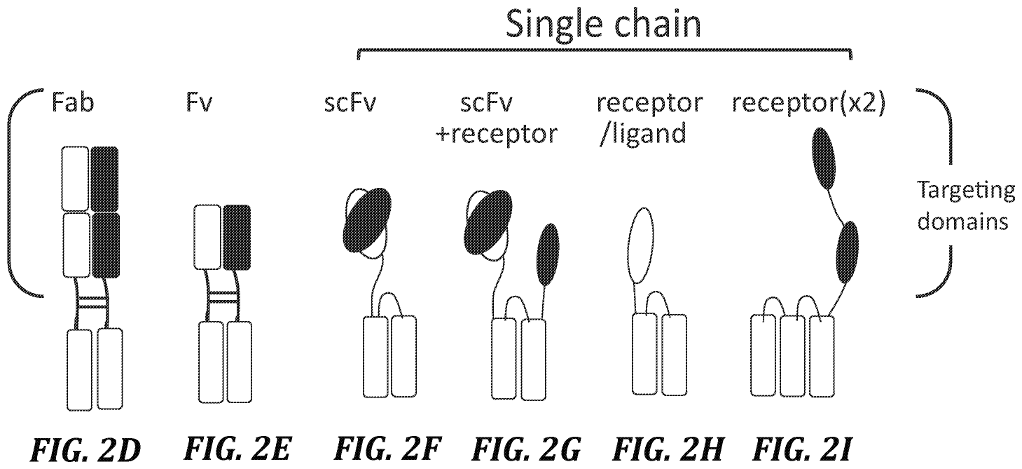

Disclosed herein are antibody-based recombinant antigen binding molecules comprising antigen-binding domains attached to the N-terminal end or C-terminal end of a core domain. The antigen-binding domains are located at 1, 2, 3, 4, 5, 6, or more sites within the antibody-based recombinant antigen binding molecule as illustrated in FIG. 2. In some embodiments, the antibody-based recombinant antigen binding molecule has one binding site located at the N-terminal end and C-terminal end of the first subunit and the N-terminal end and C-terminal end of the second subunit. In some embodiments, the antibody-based recombinant antigen binding molecule has two binding sites located at the N-terminal end and C-terminal end of the first subunit and the N-terminal end and C-terminal end of the second subunit. In some embodiments, the antibody-based recombinant antigen binding molecule has more than two binding sites located at the N-terminal end and C-terminal end of the first subunit and the N-terminal end and C-terminal end of the second subunit.

These antigen-binding domains can include various structures and classes such as scFv, Fv, F(ab′)₂, a Fab, T cell receptor-alpha (TCR-alpha) and T cell receptor-beta (TCR-beta) variable domains, T cell receptor-gamma (TCR-gamma) and T cell receptor-delta (TCR-delta) variable domains, or a VHH-based single domain. The binding domain can be a domain originating from a natural protein domain, such as a native cell receptor, a cellular ligand, or a secreted protein. For example, a cytokine, an immune checkpoint, (i.e., PD-1), a chemokine, a membrane protein that stimulates T cells, NK cells (i.e., CD137L) or dendritic cells (i.e., CD40L), a natural killer receptor (i.e., NKG2D), a gamma/delta T cell receptor (for example, targeting MR1), or another immune regulator such as CD2, CD80, CD40 or CD137 as illustrated in FIG. 2. In some embodiments, the immune checkpoint protein is PD-1, Lag3, B7H3, TIGIT, TIM3, SigLec15, or TREM2. In some embodiments, the cytokine is IL15, IL7, or IL12. In some embodiments, the chemokine is CXCL10, CCL19, or CCL20. In some embodiments, the immune cell activator is CD137, OX40, CD40, ICOS, CD2, CD28, CD3 or their ligands. In other embodiments, the natural killer receptor is NKG2D, NKp44, NKp46, NKp30, or NKRP1A.

In some embodiments, the antigen binding domain is specific for binding to a tumor associated antigen, a tumor specific antigen, a death domain (DD) receptor, a leukocyte antigen, an effector cell receptor, a cytokine receptor, a Toll-like receptor (TLR), a phagocytic receptor, or an immune checkpoint protein. In some embodiments, the tumor associated antigen is Trop2, Her2, B7H3, FAPa, CD19, CD22, MR1, CD1d, MICA/B, ULBP, Claudin18.2, MARCO, GPC3, Galectin-9, or NKp44L. In some embodiments, the DD receptor is DR4, DR5, DR6, or EDAR. In other embodiments, the effector cell receptors are TCRs, NK receptors, CD137, OX40, CD40, ICOS, CD2, CD28, phagocytic receptors, or TLRs. In some embodiments, the cytokine receptor is IL15, IL12, or IL7. In still other embodiments, the immune checkpoint target is PD-1, PD-L1, Lag3, B7H3, TIGIT, TIM3, TREM2, Siglec15, phosphatidylserine (PS), or CTLA-4.

In some embodiments, the antibody-based recombinant antigen binding molecules specifically bind to tumor targets. In some embodiments, the tumor targets can include tumor associated antigens such as Trop2, Her2, B7H3, MARCO, FAPa, Siglec15, CD19, EpCAM, GPC3, Claudin18.2, galectin-9, cadherins, CEACAMs, and the like. In some embodiments, the tumor targets can include stress antigens, such as EphA2, phosphatidylserine (PS), Annexins, F1-ATPase, MR1, CD1d, ULBPs, MICA/B, and the like. In some embodiments, the tumor targets can include natural cytotoxicity receptor ligands such as NKp44L/21spe-MLL5, PCNA, B7-H6, and the like.

In some embodiments, additional binding domains can be linked to the N-terminus of the antigen binding molecule. For example, in some embodiments the additional binding domains can be linked to the N-terminus of the IL15 alpha receptor domain or to IL15 as illustrated in FIG. 2. In other embodiments, the additional binding domain can be linked to the N-terminus of the NKG2D dimer as illustrated in FIG. 2. Additional binding domains can confer a second mechanism of activity, or greater tumor biodistribution (greater avidity for target cells).

In some embodiments, the N-terminal Fab (or TCR-based) antigen-binding domains can be the same, i.e., symmetrical Fab domains. In some embodiments, the N-terminal Fab (or TCR-based) antigen-binding domains can be different, i.e., asymmetrical Fab domains and enable specific and distinct Fab heterodimerization.

In some embodiments, the C-terminal Fc binding domain comprises 2 antigen-binding domains as illustrated in FIG. 2. In some embodiments, the C-terminal Fc binding domain comprises 2 antigen-binding domains, wherein the antigen-binding domains are different. In some embodiments, the C-terminal Fc binding domain comprises 2 antigen-binding domains, wherein the antigen-binding domains are the same. In still other embodiments, the C-terminal Fc binding domain comprises 1 antigen-binding domain. In some embodiments, the C-terminal Fc binding domain comprises 1 antigen-binding domain, wherein the antigen-binding domain is attached to the C-terminal end of the first subunit, the C-terminal end of the second subunit, or the C-terminal end of the third subunit.

In some embodiments, the TetrAx and CHAx molecules can target effector cell receptors or ligands including CD3, CD137, CD28, CD2, OX40, CD40, ICOS, CD137L, OX40, ICOSL, CD11b, CD11c, or CD16a. In some embodiments, the TetrAx, STaT, and CHAx molecules can target tumor receptors including DR5, DR4, DR6 or EDAR. In some embodiments, the TetrAx and CHAx molecules can target effector cell receptors including cytokine receptors IL15, IL12, and IL7. In some embodiments, TetrAx and CHAx can target effector cell receptors including NK receptors such as NKG2D, NKp44, NKRP1A, NKp80, or NKp46. In still other embodiments, the TetrAx and CHAx molecules can target effector cell receptors including immune check point inhibitors such as PD-1, PD-L1, B7H3, Siglec15, TIGIT, phosphatidylserine (PS), and CTLA4. In some embodiments, the TetrAx and CHAx molecules can target effector cell receptors including receptors that are involved in modifying a tumor microenvironment (TME), such as CD137, CD206, CD11b/CD18, FAPa, phosphatidylserine (PS), or TREM-2. In some embodiments, the TetrAx and CHAx molecules can also target effector cell receptors including phagocytic receptors such as, CD11b/CD18, MER, P3 integrins, LRP1, TREM2, or CD11c/CD18. In still other embodiments, the TetrAx and CHAx molecules can be developed to treat infectious diseases and possess both viral neutralization and vaccinal activity. In some embodiments, the viral target proteins can include E protein, hemagglutinins, and SARS Cov-2 Spike protein (receptor binding domain). For example, a multispecific TetrAx can bind to Cov-2 Spike protein, CD11b/c and CD40 to facilitate phagocytosis by dendritic cells and macrophages and efficiently stimulate an anti-Cov-2 immune response. In still other embodiments, the TetrAx and CHAx molecules can be developed to treat Alzheimer's Disease or other neurodegenerative diseases. In some embodiments, multispecific TetrAx and CHAx molecules can bind and support phagocytosis of amyloid-β oligomers, amyloid-β-lipoprotein complexes, phospho-Tau proteins, or alpha-synuclein, and stimulate neurogenesis. For example, a multispecific TetrAx specific for Trem2 ligands, CD11b/c and possessing a linked BDNF can target to plaques to facilitate BDNF induced neurogenesis and amyloid-β oligomers phagocytosis or clearance (Konishi H, Kiyama H. Microglial TREM2/DAP12 Signaling: A Double-Edged Sword in Neural Diseases. Front Cell Neurosci. 2018 Aug. 6; 12:206).

In some embodiments, the MCAR binding domain or cytoplasmic domain can express a PA2 ribosomal skip peptide resulting in membrane localization of, for example, a decoy immune checkpoint such as PD-1 possessing a transmembrane domain but lacking a cytoplasmic signaling domain, a regulator of tumor microenvironment such as CD206, CD11b/CD18 agonist, or TREM2 decoy that can stimulate or support macrophage M2 to M1 transition, or an immune cell activator such as CD137L that can stimulate CD8 T cell and NK cell activation (Deczkowska A, Weiner A, Arnit V The Physiology, Pathology, and Potential Therapeutic Applications of the TREM2 Signaling Pathway. Cell. 2020 Jun. 11; 181(6):1207-1217). In some embodiments, one, two, or three CD137L monomers can be linked to a TetrAx molecule (FIG. 2); CD137L is a TNF superfamily ligand naturally forms a trimer which binds at three sites to its trimeric TNF superfamily receptor to stimulate signaling. However, multiple TetrAx molecules possessing one or two monomers can bind to its target tumor antigen to enable avid and functional presentation of CD137L to CD137 expressed on effector cells such as T cells or NKs. Similarly, a TetrAx molecule possessing 1, 2, or 3 TRAIL monomers can be presented in a multivalent manner (following binding to a tumor target antigen, such as TROP2, or to Fc receptors) such that it can bind to tumor cell DR5 or DR4 and stimulate tumor apoptosis. The requirement for 2 or 3 TetrAx molecules to be presented on a tumor cell can localize activity to a TME and enhance safety.

In some embodiments, the MCAR binding domain or cytoplasmic domain can express a cleaved bispecific T cell engager (BiTE). For example, the MCAR vector can encode (in the MCAR cytoplasmic domain) for a CD3×TAA (tumor associated antigen) specific BiTE with an N-terminal ribosomal skip sequence (e.g., PA2) and a signal peptide such that the BiTE will be secreted. In some embodiments, if the CAR is expressed in NK cells, the BiTE will induce a second mechanism of tumor cytotoxicity-redirected T cell mediated cytotoxicity.

In some embodiments, MCAR can express one or more ligands for receptors comprising death domains that are expressed on tumor cells such as DR4, DR5, DR6, TNFR1, and EDAR. These ligands, including TRAIL, TNFa, APP, and EDA1, can be constitutively expressed, or for greater safety, induced following contact of MCAR or another CAR with a target tumor cell antigen. Induction can be stimulated through CAR endo-domain signaling that activates transcription factors (NFAT or NF-kb) that bind transcription response elements, in the integrated CAR vector, 5 prime of a minimal promoter that regulates their expression. Alternatively, to address safety, these ligands can be expressed transiently in MCAR cells by electroporation of mRNA encoding these ligands. In some embodiments, extracellular vesicles (EV) produced from cells expressing MCAR and death receptor ligands can target cytotoxic activity to tumor cells. Both CAR and death receptor ligands can be fused to proteins that localize to EV. These EV, and specifically exosomes, can extend the duration of anti-tumor activity beyond that of CAR cells that are expressed transiently following mRNA electroporation. Moreover, such vesicles can be manufactured and administered as a purified off-the-shelf therapeutic.

In still other embodiments, the MCAR intracellular domain can originate from a TCR such as CD3ζ. In some embodiments, the MCAR intracellular domain can originate from various TCRs. In some embodiments, the MCAR intracellular domain can originate from co-stimulatory receptors, including CD137, CD40, ICOS, CD2, DAP10, and DAP12. In some embodiments, the CAR intracellular domain can originate from cytokine receptors, including IL15, IL7, IL18, and the like.

In some embodiments, the MPCAR intracellular domain can originate from phagocytic receptors, including CR1, CD11b/CD18, CD11c/CD18, aVb3, aVb5, LRP1, MER, and AXL to support phagocytosis when expressed in monocytes, macrophages, dendritic cells, microglial cells and NK cells. In some embodiments, the MPCAR intracellular domain can originate from scavenger receptors.

In some embodiments, the MPCAR binding domain can comprise a binding domain for phosphatidylserine (PS). In some embodiments, various stresses, such as hypoxia due to stroke, virus infection, toxic amyloid-β and phospho-Tau deposition, and excitotoxic Ca²⁺ influx, can lead to activation of membrane scramblases and PS exposure on living cells (Lemke 2019). In some embodiments, MPCAR, expressed in phagocytic cells and comprised of PS binding domains that originate from various receptors, including β2GP1, milk fat globule-EGF factor 8 (MFGE8) discoidin-like domain, CD300b, BAL1, TIM1, TIM3, TIM4, or Stabilin 2 can target and phagocytose stressed cells and alleviate their pathologic activity. In some embodiments, MPCAR can be comprised of binding domains such as CD1b integrin-domain to target cells bearing cell surface complement components such as C3d. In still other embodiments, the MPCAR binding domain can express a cleaved BiTE, wherein the MPCAR vector can encode for a cytotoxic molecule such as a CD3×TAA (tumor associated antigen) specific BiTE with an N-terminal PA2 sequence such that the BiTE will be secreted. In some embodiments, if the MPCAR is expressed in NK cells, monocytes or macrophages, the BiTE will induce a second mechanism of tumor cytotoxicity-redirected T cell mediated cytotoxicity. In still other embodiments, MPCAR can be developed to treat Alzheimer's Disease or other neurodegenerative disease. In some embodiments, MPCAR can bind and support phagocytosis of amyloid-β oligomers, amyloid-β-lipoprotein, phospho-Tau proteins and/or alpha-synuclein.

In some embodiments, MPCAR can be comprised of TREM2, LRP1 and/or scFv domains that bind amyloid-β oligomers, amyloid-β-lipoprotein, phospho-Tau proteins and/or alpha-synuclein. In some embodiments, MPCAR can possess more than one TREM2, LRP1, or svFv domain to enable multivalent or higher avidity binding to amyloid-b-lipoproteins to increase the efficiency of its removal from the circulation. In still other embodiments, other CAR designs can possess more than one TREM2, LRP1, or svFv domain to enable multivalent or higher avidity binding to amyloid-b-lipoproteins to increase the efficiency of its removal from the circulation.

Alzheimer's disease can be caused by A∂-lipoprotein produced from liver that enters the circulation and then brain via leaky vessels (Lam V, Takechi R, Hackett M J, Francis R, Bynevelt M, Celliers L M, Nesbit M, Mamsa S, Arfuso F, Das S, Koentgen F, Hagan M, Codd L, Richardson K, O'Mara B, Scharli R K, Morandeau L, Gauntlett J, Leatherday C, Boucek J, Mamo J C L. Synthesis of human amyloid restricted to liver results in an Alzheimer disease-like neurodegenerative phenotype. PLoS Biol. 2021 Sep. 14; 19(9):e3001358. doi: 10.1371/journal.pbio.3001358. PMID: 34520451; PMCID: PMC8439475). It is possible that previous clinical studies may have used Aβ antibodies that don't efficiently bind the Aβ-lipoprotein complex and that e.g., TREM2, LRP1, or scFv generated to bind this complex will be more efficacious. See SEQ ID NOs: 49 and 50. Moreover, multivalent TetrAx or MPCAR molecules can enable avid binding to Aβ-lipoprotein complexes.

In some embodiments, MPCAR can express one or more ligands for receptors comprising death domains that are expressed on tumor cells such as DR4, DR5, DR6, TNFR1, and EDAR. These ligands can be constitutively expressed, or for greater safety, induced following contact of MPCAR or another CAR with a target tumor cell antigen. Induction can be stimulated through CAR endo-domain signaling that activates transcription factors (NFAT or NF-kb) that bind transcription response elements, in the integrated CAR vector, 5 prime of a minimal promoter that regulates their expression. Alternatively, to address safety, these ligands can be expressed transiently from transfected mRNA. In some embodiments, extracellular vesicles (EV) produced from cells expressing MPCAR and death receptor ligands can target cytotoxic activity to tumor cells. Both CAR and death receptor ligands can be fused to proteins that localize to EVs. These EVs, and specifically exosomes, can extend the duration of anti-tumor activity beyond that of transiently expressed CARs. Moreover, such vesicles can be manufactured using different cell types and administered as a purified off-the-shelf therapeutic.

Certain specific embodiments for MCAR and MPCAR are shown in Table 2.

TABLE 2
lists specific examples of MCAR and MPCAR antibody-based
recombinant antigen binding receptor molecules.

| | | Structural Features of chains possessing | SEQ |
|---|---|---|---|
| | | either MCH1 or CL Monomers (from N- | ID |
| Format | Specificity | to C-terminus) | NO: |
| MCAR | TROP2 × | CL: RS7Vk, CL, linker, CD8 stalk & | 33 |
| | PDL1 | tm, P2A, PD1(decoy) | |
| | | CH1: RS7Vh, MCH1wt, CD8 stalk & | 34 |
| | | tm, CD137, CD3z | |
| MCAR | TROP2 × | CL: RS7Vk, linker, CL, CD8 stalk & | 35 |
| | CD137 × | tm, P2A, CD137L tm | |
| | PDL1 | CH1: RS7Vh, linker, MCH1wt, CD8 | 36 |
| | | stalk & tm, CD137, CD3z, P2A, | |
| | | PD1tm(decoy) | |
| MCAR | TROP2 × PtS/ | CL: RS7scFv, linker, CL, CD8 stalk & | 37 |
| | Galectin9 × | tm, PA2, CD137L | |
| | CD137 | CH1: TIM3(decoy), linker, MCH1wt, | 38 |
| | | CD8 stalk & tm, CD137, CD3z | |
| MCAR | MR1 × | CL: Vγ, CL, CD8 stalk, CD8tm, PA2, | 39 |
| | CD155(Tigit | TIGIT(decoy) | |
| | decoy), | CH1: Vδ, MCH1, CD8 stalk, CD8tm, | 40 |
| | CD137 | CD137, CD35, PA2, CD137L | |
| MPCAR | MR1 × | CL: Vγ, CL, CD8-stalk & tm, CD137, | 41 |
| | Chemokine | CD3ζ, PA2 CXCL10 | |
| | Rc, PDL1 | CH1: V8, MCH1, CD8-stalk & tm, | 42 |
| | | CD18, PA2, PD1(decoy) | |
| MPCAR | Trop2 | CL: hRS7Vk, CL, CD8-stalk & tm, | 43 |
| | | CD11b, CD3ζ | |
| | | CH1: hRS7Vh, MCH1, CD8-stalk & tm, | 44 |
| | | CD18, CD137 | |
| MPCAR | Trop2 × | CL: hRS7Vk, CL, CD8-stalk & tm, | 45 |
| | Chemokine | CD137, CD3, PA2, CXCL10 | |
| | Rc | CH1: hRS7Vh, MCH1, CD8-stalk & tm, | 46 |
| | | LRP1t | |
| MPCAR | PtS × TROP2 | CL: TIM4, CL, CD8-stalk & tm, LRP1t, | 47 |
| | | CH1: RS7scFv, MCH1, CD8-stalk & tm, | 48 |
| | | CD137, CD3ζ | |
| MPCAR | Amyloid-9 | CL chain: TREM2, linker, TREM2, | 49 |
| | (trivalent) | linker, CL, CD8-stalk & tm | |
| | | CH1 chain: TREM2, linker, MCH1, | 50 |
| | | CD8-stalk & tm, LRP1t | |

### 2. Core Domain

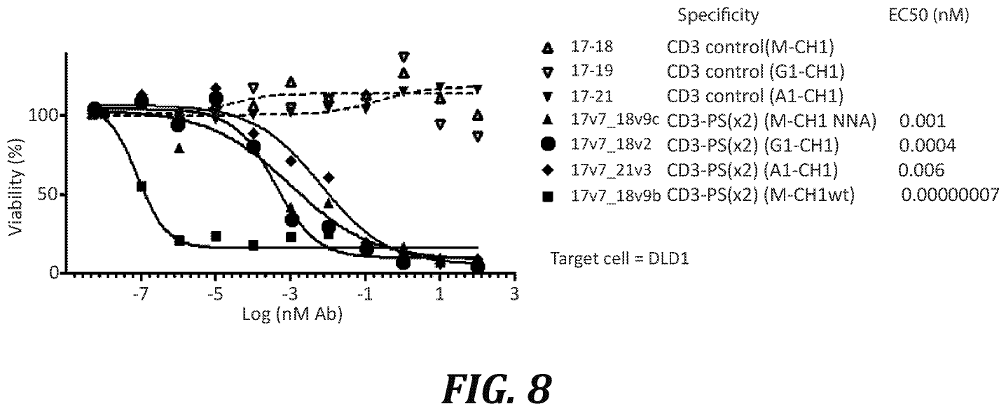

Disclosed herein are antibody-based recombinant antigen binding molecules comprising a core domain. In some embodiments, the basis of the core domains is the interaction between the CL domain and the Ig-M/A/G₂/G₃ CH1 domain to support efficient heterodimerization of the TetrAx subunit pairs or the interaction between an additional CH3-CH3 dimer comprising a knob-into-hole to enhance heterodimerization of the CHAx subunit pairs as set forth below. In some embodiments, the basis of the TetrAx core CH1 isotype is to support greater functional activity (FIG. 8). In some embodiments, the TetrAx core domain comprises a first subunit, the first subunit comprising from the N-terminus to the C-terminus, a CL domain, an immunoglobin hinge, and an IgG Fc domain comprising a CH2 domain and a CH3 domain; and a second subunit comprising from the N-terminus to the C-terminus, a Ig-M/A/G₂/G₃ CH1 domain, an immunoglobin hinge, and an IgG Fc domain comprising a CH2 domain and a CH3 domain, wherein the interaction between the CL domain and the Ig-M/A/G₂/G₃ CH1 domain drives more efficient heterodimerization of the first subunit with the second subunit. In some embodiments, the TetrAx CH3 domain can include a disulfide bridge to further stabilize the heterodimer. In some embodiments, the CH3 domain can include a knob-into-hole modification to further stabilize the heterodimer.

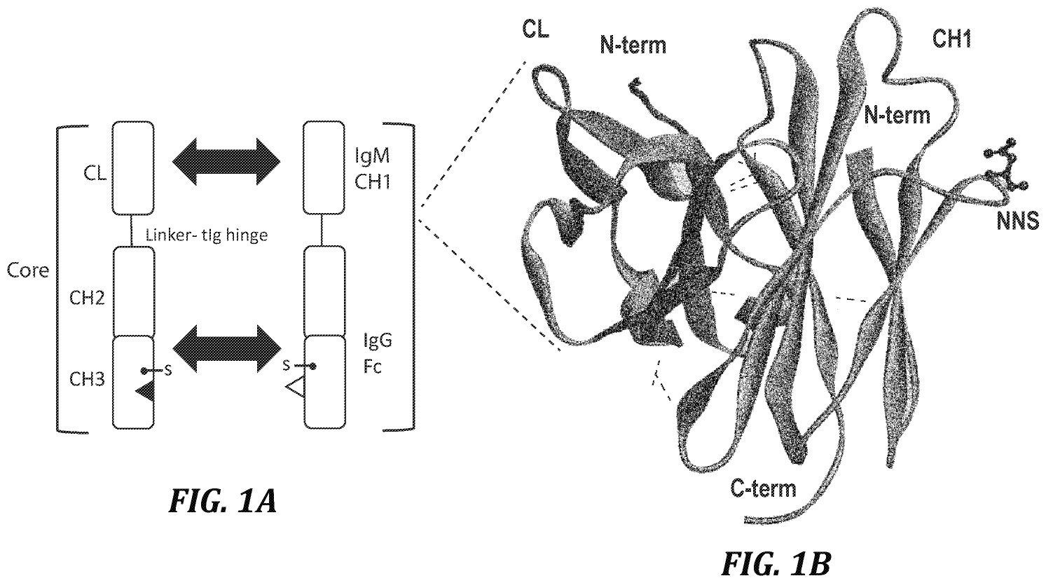

In some embodiments, the TetrAx core comprises a first subunit, the first subunit comprising from the N-terminus to the C-terminus, a CL domain; a second subunit, the second subunit comprising from the N-terminus to the C-terminus, an Ig-M/A/G₂/G₃ CH1 domain, an Fc domain comprising an immunoglobin hinge region, a CH2 domain, and a CH3 domain; a third subunit, the third subunit comprising from the N-terminus to the C-terminus, a CL domain, an Fc domain comprising an immunoglobin hinge region, a CH2 domain, and a CH3 domain; and a forth subunit, the fourth subunit comprising from the N-terminus to the C-terminus, an Ig-M/A/G₂/G₃ CH1 domain, wherein the interaction between the CL domain in the first subunit and the CH1 domain in the second subunit, and the CL domain in the third subunit and the CH1 domain in the fourth subunit drives more efficient heterodimerization of the four subunit molecule (FIG. 1).

In some embodiments, a linker can be inserted between the CH1 and the Ig hinge sequence and the N-terminal end of the Ig hinge can be truncated (Table 3). In some embodiments, the CH3 domain can include a disulfide bridge to further stabilize the heterodimer. In some embodiments, the CH3 domain can include a knob-into-hole modification to further stabilize the heterodimer.

In some embodiments, the TetrAx core comprises a first subunit, comprising from the N-terminus to the C-terminus, a CL domain; a second subunit, comprising from the N-terminus to the C-terminus, an Ig-M/A/G₂/G₃ CH1 domain, an Fc domain comprising an immunoglobin hinge, and a CH2 domain and a CH3 domain; and a third subunit, comprising from the N-terminus to the C-terminus a linker, an Fc domain comprising an immunoglobin hinge, a CH2 domain, and a CH3 domain, wherein the interaction between the CL domain and the Ig-M/A/G₂/G₃ CH1 domain supports more efficient heterodimerization of the three subunit molecule. In some embodiments, a linker can be inserted between the CH1 and the Ig hinge sequence and the N-terminal end of the Ig hinge can be truncated (Table 3). In some embodiments, the CH3 domain can include a disulfide bridge to further stabilize the heterodimer. In some embodiments, the CH3 domain can include a knob-into-hole modification to further stabilize the heterodimer.

In some embodiments, the MCAR and MPCAR core domains comprise a first subunit, comprising from the N-terminus to the C-terminus, a CL domain, a linker and/or a stalk, a transmembrane domain, and an intracellular domain; and a second subunit comprising an Ig-M/A/G₂/G₃ CH1 domain, a linker and/or stalk, a transmembrane domain, and an intracellular domain, wherein the interaction between the CL domain and the Ig-M/A/G₂/G₃ CH1 domain drives heterodimerization of the two-subunit molecule. In some embodiments, an intracellular signaling motif is located on the intracellular tail of the first subunit. In some embodiments, an intracellular signaling motif is located on the intracellular tail of the second subunit. In still other embodiments, the intracellular signaling motif is located on the intracellular tail of the first subunit and the second subunit.

In some embodiments, the coding regions of the MCAR or MPCAR molecules can also encode a protein binding domain, which can be released from an ectodomain or cytoplasmic region via ribosomal skip. In some embodiments, the releasable protein binding domain can be a transmembrane protein. In some embodiments, the releasable protein binding domain can be a secreted as an antibody or native other protein. In some embodiments, the releasable protein binding domain can be an inhibitor of an immune checkpoint (decoy) such as PD-1, Tigit, TREM2 or TIM3. In other embodiments, the releasable protein binding domain can be an activator of T cells (CD8) and/or NK cells such as an agonist specific for CD137, OX40, CD40L, ICOS, CD28, and CD2. In some embodiments, the releasable protein binding domain can be an activator of dendritic cells to stimulate and anti-tumor response such as an agonist specific for CD137L, OX40L, CD40, ICOSL, and CD80. In some embodiments, the releasable protein binding domain can be a small bispecific tumor cytotoxic antibody, such as a tandem scFv targeting tumor antigen and CD3 or DR5. In still other embodiments, the releasable protein binding domain can be a cytokine, such as IL15 to activate T and NK cells. In some embodiments, the releasable protein binding domain can be or a chemokine, such as CXCL21 to recruit lymphoid or NK cells.

Disclosed herein are CHAx cores to create two subunit antibody-based recombinant multispecific antigen binding molecules. In some embodiments, CHAx cores are comprised of an Fc region with two CH3 dimers. The high affinity CH3-CH3 interaction (Table 4) increases the overall binding strength between the core subunits. In some embodiments, each CHAx CH3 dimer comprises a knob-into-hole to enhance correct heterodimerization. In still other embodiments, each CHAx subunit is comprise of one CH3 domain with a knob and a second CH3 domain with a hole such that the heterodimeric core contains CH3 knobs in opposite orientation to eliminate the formation of hole-hole homodimers. In some embodiments, the second CH3 domain is connected with a short linker to the first CH3 domain's C-terminus. In some embodiments, the second CH3 domain is connected with a short linker to the CH2 domain's N-terminus. In other embodiments, the second CH3 domain is connected with a short linker to the Ig hinge's N-terminus. In some embodiments, the N-terminus of the Ig hinge can be truncated by 2 to 9 amino acids. In some embodiments, the knob can be located on either the first CH3, or the second CH3 of the first subunit and the hole is located on the corresponding first CH3, or second CH3 of the second subunit. In still other embodiments, the hole can be located on either the first CH3, or the second CH3 of the first subunit and the knob is located on the corresponding first CH3, or second CH3 of the second subunit.

### 3. Linker Domain

Disclosed herein are TetrAx and CHAx antibody-based recombinant antigen binding molecules comprising a linker domain. In some embodiments, the CH1-CL domains are each operatively associated to a CH2 domain and a CH3 domain through an immunoglobin linker. In some embodiments, the linker connects the C-terminus of the CL domain or the C-terminus of the CH1 domain to the N-terminus of CH2 domain. In some embodiments, the linker can be a flexible glycine-rich linker that is typically 3 to 8 residues in length. In some embodiments, the linker can be 3 to 9 amino acids in length. In some embodiments, the N-terminus of the Fc IgG1 hinge can be truncated by 1 to 9 residues. In some embodiments, the TetrAx molecules can be further modified with engineered flexible glycine-rich or rigid proline-rich peptide linkers to position binding domains for optimal ligand binding as shown, below, in Table 3.

TABLE 3
lists examples of modification to the TetrAx Fc hinge.

| | | |
|---|---|---|
| 1 | Chain 1 CL: RGECGGGAPGGGDKTHTCPPC ... | SEQ ID NO: 88 |
| | Chain 2 CH1: DVPLGGGAPGGGDKTHTCPPC ... | SEQ ID NO: 89 |
| 2 | Chain 1 CL: RGECGGGAPGGDKTHTCPPC ... | SEQ ID NO: 90 |
| | Chain 2 CH1: DVPLGGGAPGGDKTHTCPPC ... | SEQ ID NO: 91 |
| 3 | Chain 1 CL: RGECGGGGDKTHTCPPC ... | SEQ ID NO: 92 |
| | Chain 2 CH1: DVPLGGGGDKTHTCPPC ... | SEQ ID NO: 93 |
| 4 | Chain 1 CL: RGESGGDKTHTCPPC ... | SEQ ID NO: 94 |
| | Chain 2 CH1: DVPLGGDKTHTCPPC ... | SEQ ID NO: 95 |
| 5 | Chain 1 CL: RGECGGDKTHTCPPC ... | SEQ ID NO: 96 |
| | Chain 2 CH1: DVPLGGDKTHTCPPC ... | SEQ ID NO: 97 |
| 6 | Chain 1 CL: RGECGGGAPGGGGGTCPPC ... | SEQ ID NO: 98 |
| | Chain 2 CH1: DVPLGGGAPGGGGGTCPPC ... | SEQ ID NO: 99 |
| 7 | Chain 1 CL: RGECPAGGGGGGTHTCPPC ... | SEQ ID NO: 100 |
| | Chain 2 CH1: DVPLPAGGGGGGTHTCPPC ... | SEQ ID NO: 101 |
| 8 | Chain 1 CL: RGECGGGGTHTCPPC ... | SEQ ID NO: 102 |
| | Chain 2 CH1: DVPLGGGGTHTCPPC ... | SEQ ID NO: 103 |
| 9 | Chain 1 CL: RGESGGTHTCPPC ... | SEQ ID NO: 104 |
| | Chain 2 CH1: DVPLGGTHTCPPC ... | SEQ ID NO: 105 |
| 10 | Chain 1 CL: RGECGGTHTCPPC ... | SEQ ID NO: 106 |
| | Chain 2 CH1: DVPLGGTHTCPPC ... | SEQ ID NO: 107 |
| 11 | Chain 1 CL: RGECGGPAGGGGGTHTCPP | SEQ ID NO: 108 |
| | Chain 2 CH1: NVPLPAGGGGGTHTCPPC | SEQ ID NO: 109 |
| 12 | Chain 1 CL: RGECGGPAGGGGGTHTCPPC ... | SEQ ID NO: 110 |
| | Chain 2 CH1: DVPLPGGPAGGGGGTHTCPPC ... | SEQ ID NO: 111 |

In some embodiments, the linker can connect the N-terminus of the VH with the C-terminus of the VL. In some embodiments, the linker can connect the C-terminus of the VL with the N-terminus of the VH. In some embodiments, the linker can connect the antigen-binding domain to the N-terminus of the CL domain. In some embodiments, the linker can connect the antigen binding domain to the N-terminus of the CH1 domain. In some embodiments, the linker can connect the antigen binding domain to the C-terminus of the Fc domain.

In still other embodiments, certain regions of the TetrAx and CHAx molecules can be connected with a linker peptide of 3 to about 16 amino acids. In some embodiments, the linker can be glycine-rich for flexibility. In some embodiments, the linker can be proline-rich for rigidity. In still other embodiments, the linker can comprise serine or threonine for solubility.

In still other embodiments, the MCAR and MPCAR CL or CH1 regions can be connected to a stalk region through a short glycine or proline rich linker. The stalk region can originate from various membrane proteins, including CD8, CD28, and glycosaminoglycans.

### 4. Fc Domain

Disclosed herein are TetrAx and CHAx antibody-based recombinant antigen binding molecules comprising an Fc domain. In some embodiments, the Fc can be selected from an IgG₁, an IgG₂, an IgG₄, or an IgA. In some embodiments, the Fc domain can be modified through amino acid substitutions that eliminate or enhance binding to Fc immunoglobulin receptors, FcgRs (Saunders K O. Conceptual Approaches to Modulating Antibody Effector Functions and Circulation Half-Life. Front Immunol. 2019 Jun. 7; 10:1296.). In some embodiments, the additional CH3 dimer in CHAx can be positioned to decrease or eliminate FcR binding. In some embodiments, amino acid mutations can be generated to increase binding to the Fc immunoglobulin receptor IIIa (FcgRIIIa) and enhance NK cytotoxicity, the amino acid mutations can be serine at position 239 is replaced with aspartic acid (S239D), the alanine residue at position 330 is replaced by leucine (A330L), and/or the isoleucine residue at position 332 is replaced by glutamic acid (I332E) (“DLE”), the serine residue at position 298 is replace with alanine (S298A), the glutamic acid at position 333 is replaced by alanine (E333A), and/or the lysine residue at position 334 is replaced by alanine (K334A) (“AAA”). In still other embodiments, the amino acid mutations L234A and/or L235A (“LALA”) can be used to decrease FcgR binding. In some embodiments, the CH3 in the Fc domain can further comprise a knob-into-hole modification to further support heterodimerization. In still other embodiments, the CH3 in the Fc domain can further comprise a disulfide bridge to further support heterodimerization. In some embodiments, the CH3 in the Fc domain can further comprise a knob-into-hole modification and a disulfide bridge to further support heterodimerization. In some embodiments, in the CHAx molecule, the additional CH3 dimer will comprise a knob-into-hole in the opposite orientation, i.e., the first CH3 on the first subunit can comprise a knob and the second CH3 on the first subunit can comprise a hole, as the first CH3 dimer to increase correct heterodimerization and prevent formation of homodimers of hole subunits.

### B. Heterodimerization and Stability

Disclosed herein are antibody-based recombinant antigen binding molecules comprising a core structure or scaffold comprising an Ig-M/A/G₂/G₃ CH1 domain on at least one subunit that binds to the CL domain on at least a second subunit, to more effectively drive heterodimerization and generate stable multispecific recombinant antigen binding molecules. Surprisingly, the affinity of IgM CH1 for CL is calculated to be substantially greater than other Ig domain interactions including, that of CH3-CH3 which is the most important stabilizing domain for most therapeutic antibodies and for knob-into-hole bispecific antibodies. Affinity is calculated using 3D structures. (Honorato R V, Koukos P I, Jiménez-Garcia B, Tsaregorodtsev A, Verlato M, Giachetti A, Rosato A, Bonvin AMJJ. Structural Biology in the Clouds: The WeNMR-EOSC Ecosystem. Front Mol Biosci. 2021 Jul. 28; 8:729513. doi: 10.3389/fmolb.2021.729513. PMID: 34395534; PMCID: PMC8356364). Prodigy estimates affinity from 3D structures with high accuracy (R=−0.73, p<0.0001; RMSE=1.89 kcal mol⁻¹). The IgM CH1-CL binding affinity is 350-fold greater than that of IgG₁ CH1-CL, which is most often used for generating mono- and bispecific therapeutic antibodies as shown below in Table 4.

TABLE 4
lists the calculated affinity and dissociation constants between the CHI
domain and the CL domain for different Ig isotypes (*estimated from
3D structures (Honorato R V, et al., Front Mol Biosci. 2021 Jul. 28;
8:729513)).

| | Binding affinity | Dissociation constant Kd | |
|---|---|---|---|
| Isotype | ΔG (kcal mol⁻¹) | (M) at 25.0° C. | PDB# |
| IgM | −14.8 | 1.4E−11 | 1DEE |
| IgA1 | −11.8 | 2.1E−09 | 5e8e |
| IgG2 | −11.7 | 2.5E−09 | 2xtj |
| IgE | −11.7 | 2.7E−09 | 2r56 |
| IgG1 | −11.3 | 4.9E−09 | 1hzh |
| IgG4 | −10.9 | 9.8E−09 | 5dk3 |
| IgG3 (mouse) | −11.9 | 2.0E−09 | 4hdi |
| CH3 (G1) | −14.5 | 2.2E−11 | 5k65 |

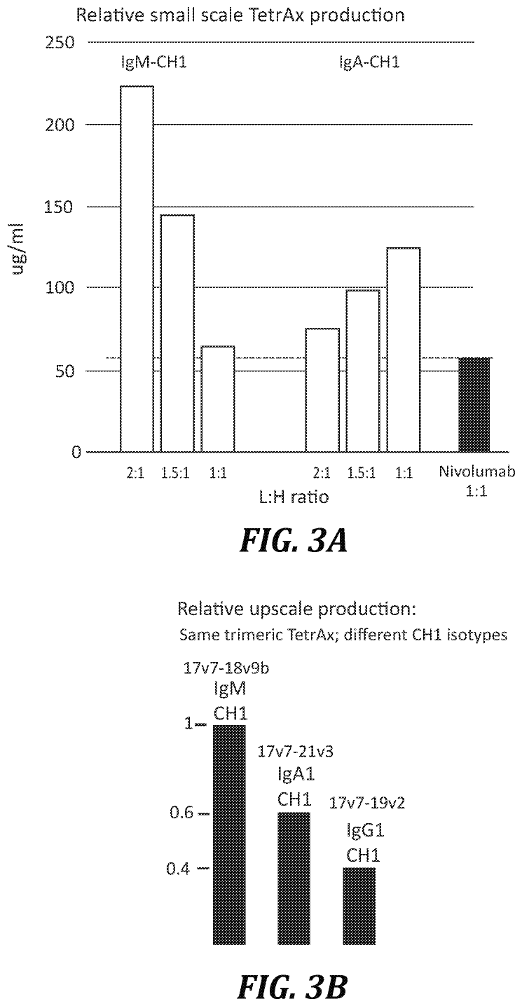

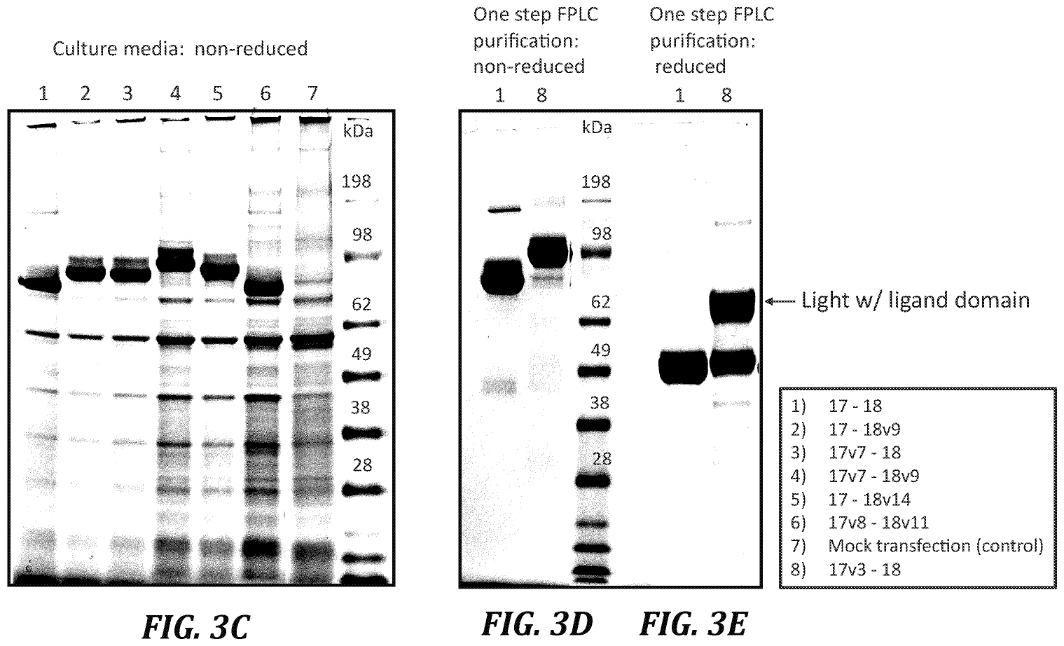

Further, the IgM CH1-CL affinity is 700-fold greater than that of IgG4 CH1-CL, which is also commonly used to generate mono- and bispecific therapeutic antibodies. Table 4. Moreover, IgG1 and IgG4 CL-CH1 binding affinity is estimated to be significantly lower than that of IgA, IgG2 and mouse IgG3 (Table 4). Incorporation of Ig-M/A/G2/G3 CH1 into the core of one subunit to bind CL of a second subunit can therefore enhance the efficiency of subunit heterodimerization and ultimately production and stability. Thus, the production of a TetrAx molecule comprising an IgM CH1 core can be greater than that of an equivalent molecule that only differs in CH1 isotype or that of a therapeutic IgG4 antibody produced at the same time in the same system (FIG. 3A).

IgG1 and IgG4 are the most commonly used isotypes for the production of monospecific and bispecific recombinant antigen binding therapeutics. TetrAx, ChAx, MCAR, and MPCAR incorporate IgM or Ig-A/G2/G3 CH1 to enhance production and stability of multispecific antigen binding molecules and higher valances that enable multiple mechanisms of activity for greater efficacy.

In other embodiments, antibody-based recombinant antigen binding molecules comprising a core structure or scaffold comprising an Ig Fc with a second CH3-CH3 dimer (CHAx) to more effectively drive heterodimerization and generate stable multispecific recombinant antigen binding molecules are disclosed. The CH3-CH3 affinity is estimated to be approximately only 1.5-fold lower than that of IgM-CH1-Cl (Table 4). Thus, a core comprised of an Fc with an additional CH3 dimer with a knob-into-hole can support heterodimerization and stability to enhance production of multispecific antigen binding molecules enabling additional mechanisms of activity.

In still other embodiments, the antibody-based recombinant antigen binding molecules' core structure can be stabilized through an interchain disulfide bond between the CL-CH1 region.

In still other embodiments, the efficiency of heterodimerization can be further enhanced through the addition of laterally interacting N-terminal binding domains that are located on opposing subunits. For example, the IL15 alpha (IL15a) receptor fragment's residues, from approximately residues 30 to 102 can be linked to the N-terminus of a CL subunit (SEQ ID NO: 31) and IL15 receptor can be linked to the N-terminus of the CH1 subunit (SEQ ID NO: 33). In some embodiments, the CL domain and CH1 domain can alternatively comprise an ectodomain fragment of NKG2D from approximately residues 51 to 216 at their N-terminus to form a ligand binding NKG2D homodimer. The stability of the homodimer can be further increased through the creation of an interdomain disulfide bond.

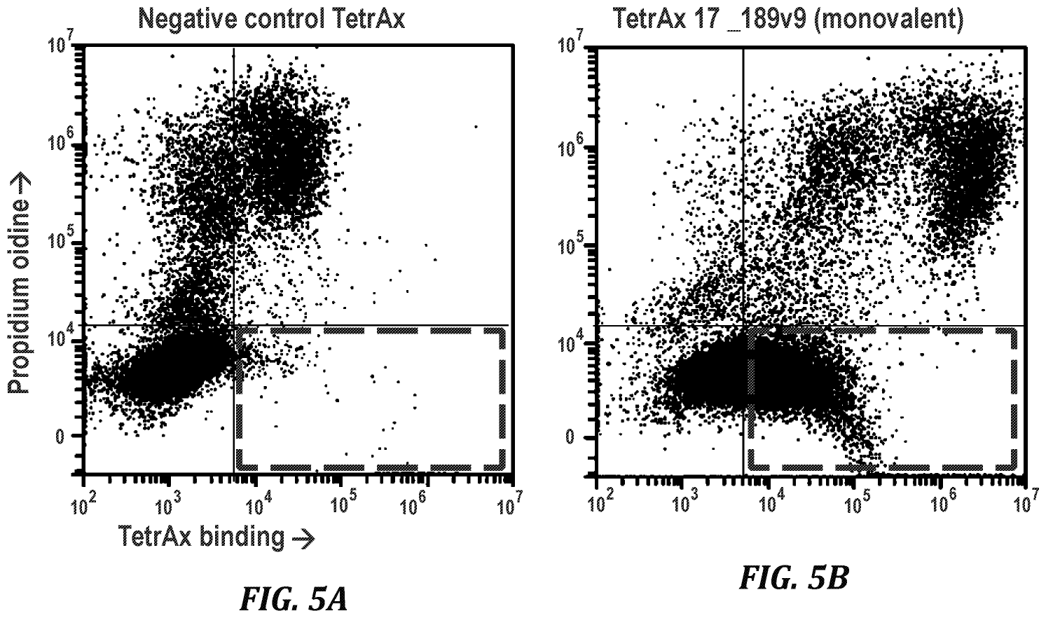

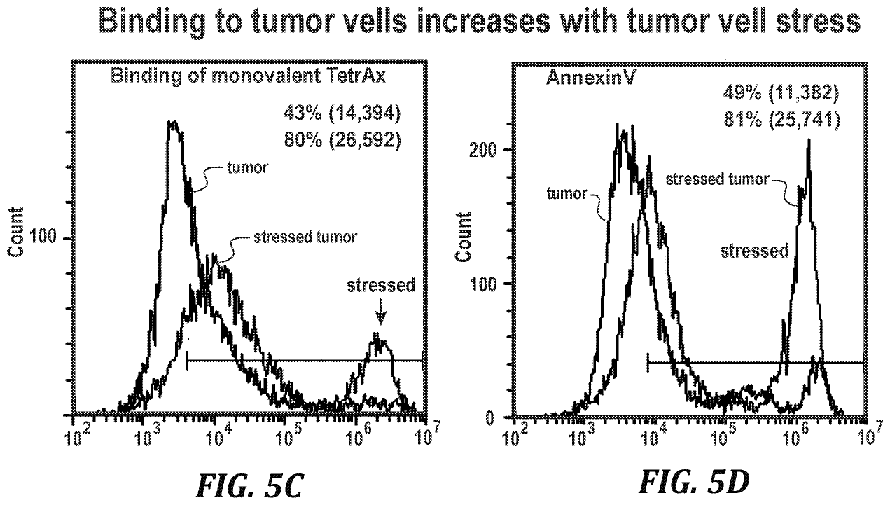

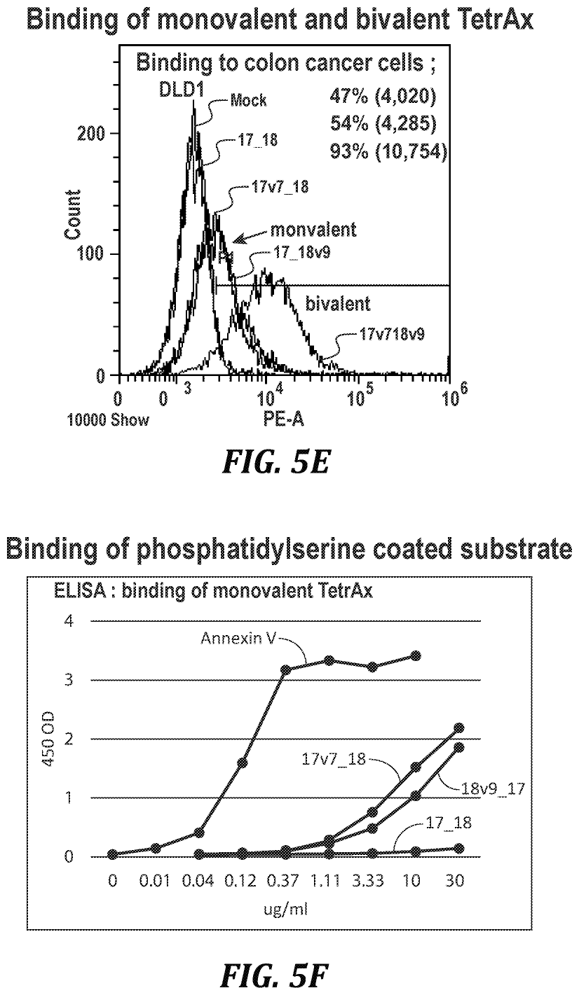

In some embodiments, the efficiency of heterodimerization can be further enhanced by laterally interacting variable domains (Ig, Fv, or TCR) with any specificity. In still other embodiments, the efficiency of heterodimerization and production can be affected by the choice of kappa or lambda CL used. For example, under the same conditions, a TetrAx can be expressed at significantly higher levels relative to the same TetrAx with a kappa that differs by only two amino acids in the CL region. In some embodiments, the efficiency of binding of the target antigen can be affected by the N-linked glycosylation of IgM CH1, which can be eliminated by amino acid substitution mutagenesis. However, the binding of a TetrAx to the T cell CD3 is not significantly affected by the presence of the proximal IgM CH1 N-linked glycosylation (FIG. 5).

In some embodiments, the TetrAx molecules' heterodimerization and stability can be driven predominantly by the high affinity IgM CH1-CL interaction and not require knob-into-hole Fc modification to further enhance heterodimerization. In some embodiments, the knob-into-hole mutations in CH3 can further contribute to the TetrAx molecules' heterodimerization and stability.

### C. Anti-Tumor Mechanism of Action

Disclosed herein are antibody-based recombinant antigen binding molecules comprising a critical and defining range of affinities for the different antigens or receptors they bind. Generally, the affinity for a tumor target antigen is relatively high (e.g., ≤5 nM) and supports biodistribution predominantly to tumors whereas the affinity for a second or third antigen is low (e.g., about 20-200 nM). In some cases, the low affinity activation is around 20-fold lower relative to the tumor target affinity.

First, antigen binding molecules having an affinity range, i.e., low and high affinities, enable greater efficacy and safety because the low affinity binding decreases binding to antigens that might be more broadly expressed, resulting in less antibody “sink” to these tissues and thus more efficient dosing. For example, binding to CD3 on T cells in peripheral blood and in lymphoid tissues. Second, the low affinity binding localizes the mechanism of action associated with these low affinity interactions to the tumor. Thus, the antibody coats tumor cells through its high affinity interaction to the tumor target. This creates a multivalent surface that is now sufficiently avid to trigger mechanisms of action associated with the lower affinity specificities. For example, low affinity but high avidity binding to T cells, natural killer cells, or phagocytic cell receptors trigger T cell or natural killer cell cytotoxicity or macrophage phagocytosis that is localized to the tumor. Furthermore, low affinity but high avidity binding to immune checkpoints (e.g., PDL1, PtS), can block immune suppression that is localized to the tumor. This enables greater safety by limiting off-tumor toxicity and greater efficacy by allowing higher more potent or effective dosing.

In some embodiments, a single multispecific TetrAx or CHAx recombinant antigen binding molecule can stimulate two or three mechanisms of action (MOAs) to enable enhanced anti-tumor activity, and greater efficacy relative to traditional antibody therapeutics. These mechanisms can include different combinations of MOAs. In some embodiments, the TetrAx or CHAx can bind to a tumor associated antigen (TAA) such as Trop2 with high affinity to support distribution to the TME; the TetrAx molecule can also bind to tumor cell antigen DR5 to induce tumor cell apoptosis; and the TetrAx molecule can also bind to PD-L1 to decrease immunosuppression within the tumor microenvironment (TME). In some embodiments, a TetrAx or CHAx can bind to CD137, IL15 receptor, or other stimulatory receptor to activate T cells and natural killer cells and support their expansion and cytotoxic activity. The binding of TetrAx or CHAx molecules to T cells and natural killer cells would not trigger their apoptosis through DR5 as its activity can be suppressed in T cells and natural killer cells. Moreover, several TNF superfamily members demonstrate specific apoptosis induction in transformed cells (Diaz Arguello O A, Haisma H J. Apoptosis-Inducing TNF Superfamily Ligands for Cancer Therapy. Cancers (Basel). 2021 Mar. 27; 13(7):1543. doi: 10.3390/cancers13071543. PMID: 33801589; PMCID: PMC8036978). Targeting the IL15 receptor would not support Treg expansion as they do not express an IL15 receptor.

In other embodiments, the MOAs include T cell or natural killer cell mediated killing and phagocytosis. In some embodiments, the MOAs include, natural killer cells killing and blockade of tumor immunosuppression. In some embodiments, MOAs include regulating tumor associated macrophages (TAMs) or cancer associated fibroblasts (CAFs) functioning in the tumor microenvironment (TME). In some embodiments, MOAs include decreasing immunosuppression and decreasing extracellular matrix survival signals. In still other embodiments, MOAs include broadly disrupting a pro-tumor stroma/microenvironment.

### D. Restricting Activity to Target Cells

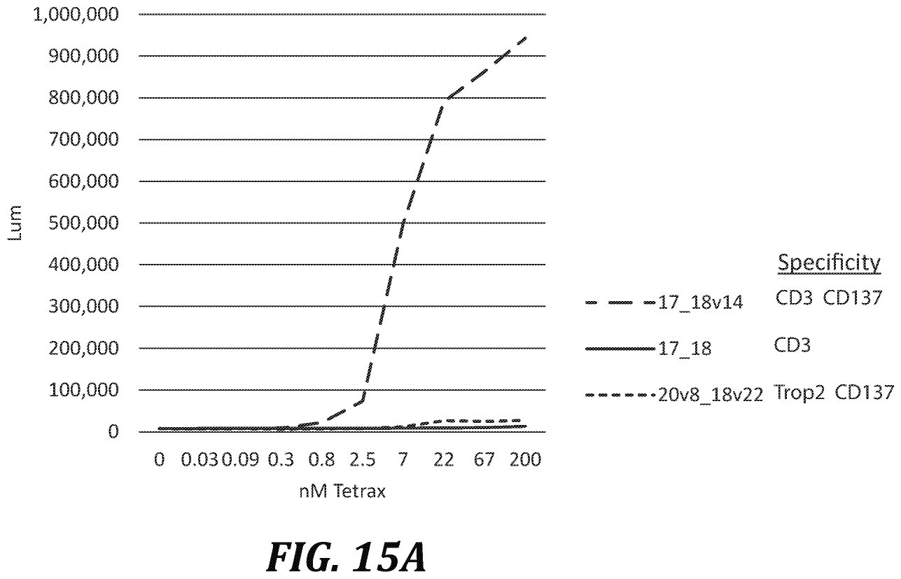

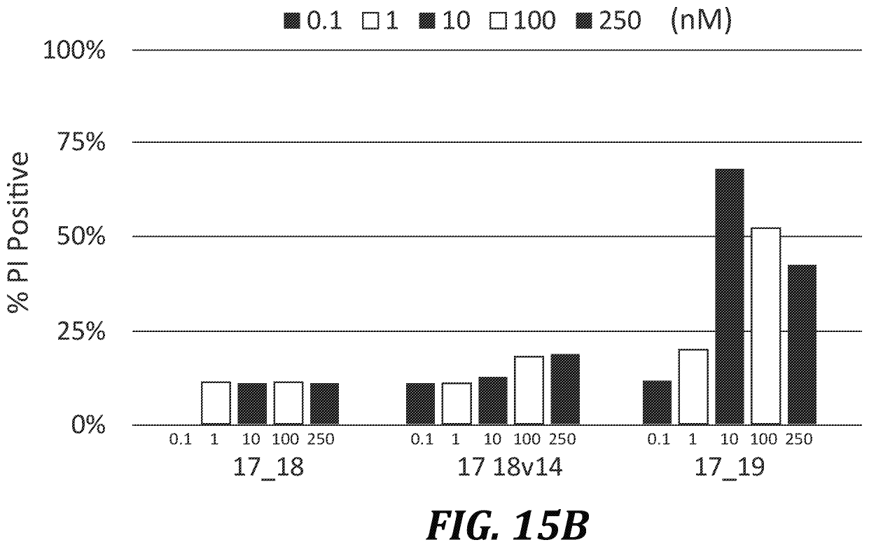

TetrAx recombinant antigen binding molecules can be designed such that their agonistic immune function is dependent on the presence of cells expressing a tumor antigen. This targeting can localize activity to a tumor microenvironment to enhance safety, enable higher dosing and greater efficacy. In some embodiments, TetrAx recombinant antigen binding molecules can possess an agonistic domain that can be one or more tumor necrosis factor superfamily (TNFSF) ligands such as CD137L, TRAIL, CD40L, CD27L, and OX40L. In addition, TetrAx recombinant antigen binding molecules can also possess a binding domain for a target antigen such as Trop2. Most TNFSF receptors require crosslinking for efficient signaling (category 2 receptors), including CD137, DR5, CD40, CD27 and OX40. Native TNFSF ligands and their receptors possess trimeric structures which engage and drive crosslinking to induce cell signaling. However, it was determined that TetrAx recombinant antigen binding molecules possessing a single TNFSF ligand domain/subunit can induce robust signaling (FIG. 15). In some embodiments, a configuration with two ligands can further increase signaling (FIG. 15). For example, in some embodiments, TetrAx recombinant antigen binding molecules were designed with a single C-terminal CD137L domain and an N-terminal target antigen binding moiety (CD3 or Trop2). When these purified TetrAx recombinant antigen binding molecules were incubated with a CD137 reporter cell line (Promega), the TetrAx recombinant antigen binding molecule induced robust signaling only when in the presence of a cell expressing the target antigen.

In other embodiments, TetrAx recombinant antigen binding molecules that lacked the target antigen binding moiety or the CD137L domain did not induce such a response. Apparently, TetrAx recombinant antigen binding molecules possessing a CD137L domain, although capable of engaging CD137, do not drive crosslinking unless they are presented in a multivalent fashion. Binding to the target cell membrane antigen results in a cell surface that can present multivalent TNFSF ligands capable of crosslinking their receptors on effector cells to induce signaling.

TetrAx recombinant antigen binding molecules can be designed to localize signaling and effector cell function or killing of tumor cells to an environment containing the target antigen. This can enable administration at higher more efficacious doses. Administration of a CD137 agonist (Urelumab), possessing a native antibody configuration, results in a dose limiting liver inflammation. To avoid liver inflammation, one company (CytomX), is developing a CD137 agonist antibody that is activated by a tumor protease. The demonstrated TetrAx recombinant antigen binding molecule target antigen dependency can localize activity to a microenvironment similar to a protease activated antibody or therapeutic, however it does not rely on sufficient expression of the protease.

Target antigen dependent TetrAx recombinant antigen binding molecules (or whole antibody format) possessing a TNFSF ligand are also advantageous relative to native antibody format in that this ligand is a single small domain capable of inducing CD137 activation. Given that native TNFSF ligands are trimeric, it is surprising that a TetrAx with a single domain is sufficient to crosslink their receptors—a prerequisite for signal transduction.

TetrAx recombinant antigen binding molecules possessing TNFSF ligands can be generated for various functions and clinical indications. For example, a TetrAx recombinant antigen binding molecule can possess more than one TNFSF ligand, e.g., CD137L and CD40L, to activate additional cell types and/or functions, resulting in a robust anti-tumor immune response. CD137 agonists can activate CD8 T cell and NK cell tumor cytotoxicity and promote monocyte M1-like transition and tumoricidal activity (Stoll A, Bruns H, Fuchs M, Völkl S, Nimmerjahn F, Kunz M, Peipp M, Mackensen A, Mougiakakos D. CD137 (4-1BB) stimulation leads to metabolic and functional reprogramming of human monocytes/Macrophages enhancing their tumoricidal activity. Leukemia. 2021 December; 35(12):3482-3496). CD40 agonists can activate dendritic cells leading to presentation of tumor neoantigens and result in a long-term tumor immune response.

Target antigens can be membrane or tumor stromal proteins. TetrAx recombinant antigen binding molecules can be designed to mediate forward or both forward and reverse signaling. For example, the target antigen can be present on tumor associated macrophages (TAMs) (e.g., CD11b, TREM2 or CD206). Targeting certain TAM antigens can resulting in transitioning of a suppressive pro-tumor microenvironment to a more inflammatory anti-tumor environment.

### E. Production

In one aspect, a method is provided for the production of antibody-derived recombinant antigen binding molecules. The method includes the step of culturing a host cell comprising at least one expression vector that can express a protein comprising a first fusion polypeptide and a second fusion polypeptide. The method further includes the step of culturing the host cell under conditions that enable heterodimerization of the first polypeptide with the second fusion polypeptide to enable expression of a stable antibody-derived recombinant antigen binding molecule. In some embodiments, the production of the antibody-derived recombinant antigen binding molecule is greater compared to production of the monoclonal antibody Nivolumab as illustrated in FIG. 3.

In some embodiments, the antibody-derived recombinant antigen binding molecule is expressed by creating two expression vectors encoding the two polypeptides. In some embodiments, the antibody-derived recombinant antigen binding molecule is expressed by creating a single bicistronic expression vector. In some embodiments, the expression vector includes a plasmid or a virus-based mechanism for integration. In still other embodiments, the expression vector comprises one or more sequences taken from SEQ ID NOs: 1-50. See Table 7.

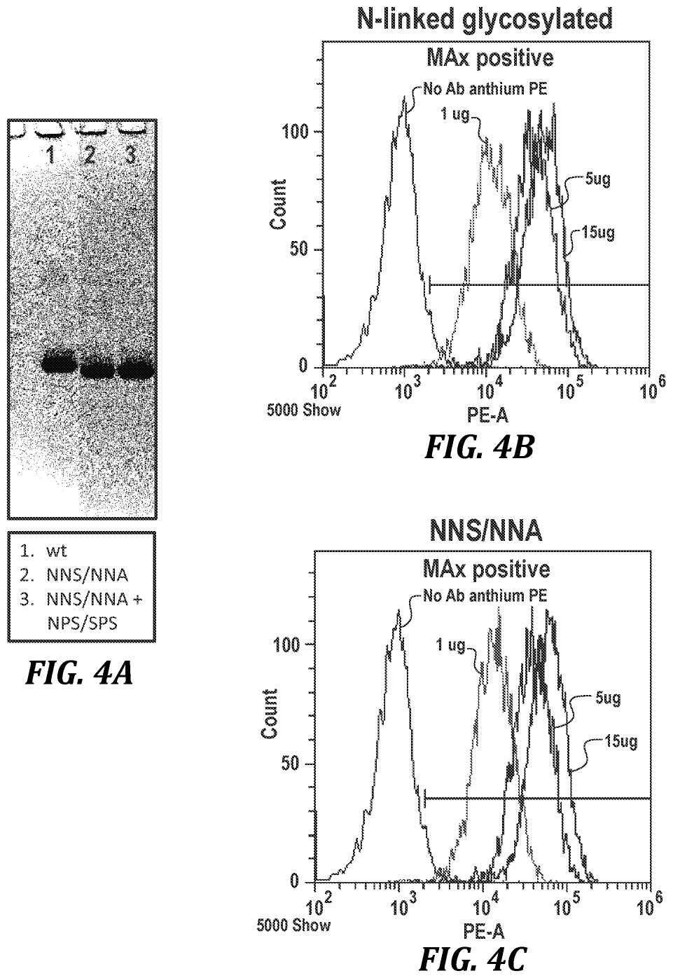

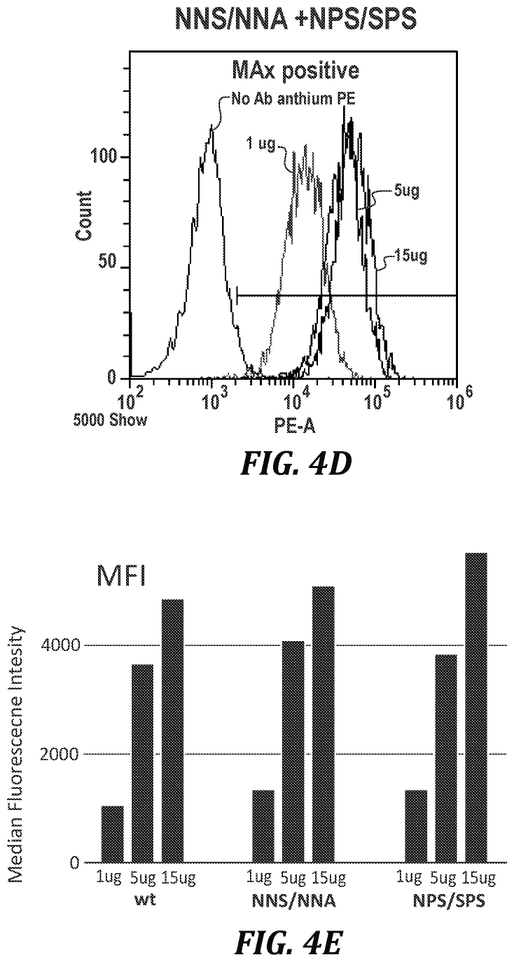

In some embodiments, the two expression vectors or single bicistronic expression vector is introduced into a mammalian host cells by transfection. In some embodiments, the mammalian host cell can be a HEK293 cell, a COS cell, or a CHO cell or some other mammalian heterologous expression system well known to a person of ordinary skill in the art. In some embodiments, the antibody-derived recombinant antigen binding molecule is purified from the culture media by Fast Protein Liquid Chromatography (FPLC) using a protein-A column following purification protocols that are well known to a person of ordinary skill in the art. In still other embodiments, FPLC can be followed with cation and/or anion exchange chromatography to increase purity. In still other embodiments, TetrAx is purified using a one-step purification protocol as illustrated in FIG. 4.

TetrAx molecules can be manufactured in vitro and administered as a recombinantly produced purified protein or TetrAx molecules can be administered as a product of an engineered cell line such as a CAR cell. For example, TetrAx molecules can be produced constitutively from T cells or NK CAR cells from an integrated lentiviral, adenoviral or transposon vector. Alternatively, TetrAx molecules can be produced under the control of an inducible promoter that is regulated by, for example, NFAT or NF-kb response elements. Production can then be induced in vivo following contact of the target antigen with a CAR whose endo-domains signal activation of NFAT or NF-kb.

Alternatively, a TetrAx molecule can be expressed transiently from, for example, a T cell or an NK cell. Thus, its mRNA can be synthesized (see Example 5) and electroporated into T cells, NK cells, CAR T cells, NK cells, or PBMCs, which are then administered to a patient. A multispecific TetrAx molecule can therefore be administered systemically, locally or secreted from lymphoid cells or CAR cells for long term or transient production.

In some embodiments, MCAR can be constitutively expressed in T cells or NK cells via an integrated vector that can be lentiviral or adenoviral vectors, transposon vectors or other recombinant expression vectors. The vector can mediate site specific integration using for example, CRISPR or TALEN technology. In some embodiments, MCAR can be expressed transiently following electroporation of mRNA encoding MCAR into T cell and/or natural killer cells; MCAR's enhanced stability, resulting in longer cell surface duration will be a significant advancement for RNA CAR that can enable greater efficacy.

In some embodiments, MCAR mRNA can be produced, for example, using a vector comprising a T7 promoter. The vector (pR) used for in vitro transcription (IVT) of MCAR (or MPCAR) can comprise two or three modified 3′UTR and a long poly A tail to stabilize mRNA and increase duration of MCAR expression in effector cells. Typically, IVT vectors comprise one or two beta-globin 3′UTR (Holtkamp S, Kreiter S, Selmi A, Simon P, Koslowski M, Huber C, Tureci O, Sahin U. Modification of antigen-encoding RNA increases stability, translational efficacy, and T-cell stimulatory capacity of dendritic cells. Blood. 2006 Dec. 15; 108(13):4009-17. doi: 10.1182/blood-2006-04-015024. Epub 2006 Aug. 29. PMID: 16940422). A second or third 3′UTR can be inserted such as that from FCGRT, LSP1, AES, or mtRNR1 to further enhance mRNA stability (Orlandini von Niessen A G, Poleganov M A, Rechner C, Plaschke A, Kranz L M, Fesser S, Diken M, Lower M, Vallazza B, Beissert T, Bukur V, Kuhn A N, Tureci Ö, Sahin U. Improving mRNA-Based Therapeutic Gene Delivery by Expression-Augmenting 3′ UTRs Identified by Cellular Library Screening. Mol Ther. 2019 Apr. 10; 27(4):824-836. doi: 10.1016/j.ymthe.2018.12.011. Epub 2018 Dec. 18. PMID: 30638957; PMCID: PMC6453560). Binding sites in pR 3′UTR for micro-RNA (miR) that can decrease mRNA stability and expression, can be mutated to further enhance MCAR and MPCAR expression (Table 5). For example, the 3′UTR in b-globin comprises target sequences that are highly complementary to miR-361 and miR-5009. These mRNA sequences can hybridize to each other as indicated in the predicted secondary structure. Substitution mutants can be generated in the vector 3′UTR such that the mRNA secondary structure can be maintained while the miRNA binding sites are disrupted. For example, the miR-361 and miR-5009 binding site sequences can hybridize to each other as indicated in the predicted secondary structure. In the predicted secondary structure, a Cysteine (C) in the miR5009 binding site forms bonds with a Guanidine (G) in the miR361 binding site. In some embodiments, substitution of the C to a G in the miR5009 site and substitution of the G to a C in the miR361 site can disrupt binding of both miRNA but maintain mRNA secondary structure and its contribution to mRNA stabilization.

In still other embodiments, MPCAR can be constitutively expressed in monocytic or macrophage cells via an integrated vector that can be a lentiviral or an adenoviral vector, a transposon vector or other recombinant expression vectors. Alternatively, MPCAR can be expressed transiently following electroporation of mRNA encoding MPCAR.

MPCAR can support more than one mechanism of anti-tumor activity and its activity can differ depending on cell type and the endo-domains that it comprises. For example, an MPCAR mRNA can be electroporated into peripheral blood mononuclear cells (PMCs) or cord blood cells that are comprised of T cells, B cells, NK cells, monocytes, and dendritic cells. In T cells, MPCAR can function as MCAR to support granzyme/perforin mediated tumor cytotoxicity. In NK cells, MPCAR can support both granzyme/perforin and phagocytic anti-tumor activity. MPCAR can also be expressed in PMNs to redirect their phagocytic and oxidative activity to enable efficient killing of hematologic tumors. In monocytes, MPCAR can primarily mediate phagocytic anti-tumor activity. MPCAR can be expressed in monocytes and macrophages expanded from cord blood. Modified, stabilized MPAR mRNA can be produced using the pR vector. MPCAR's enhanced stability, resulting in longer cell surface duration will be a significant advancement for RNA CAR that can enable greater efficacy.

Embodiment 1. An antibody-derived recombinant antigen binding molecule comprising a scaffold with at least a first subunit having an N-terminus and a C-terminus, at least a second subunit having an N-terminus and a C-terminus, at least a third subunit having an N-terminus and a C-terminus, and at least a fourth subunit having an N-terminus and a C-terminus; and the first subunit, comprising from the N-terminus to the C-terminus, a first antigen binding domain (A) and a CL light chain constant region of an Igκ or an Igλ; the second subunit, comprising from the N-terminus to the C-terminus, a second antigen binding domain (B), a CH1 heavy chain constant region, an immunoglobulin hinge region, a heavy chain Fc domain from an IgG, and a third antigen binding domain (C); the third subunit, comprising from the N-terminus to the C-terminus, a fourth antigen binding domain (D), a CL light chain constant region of an Igκ or an Igλ, an immunoglobulin hinge region, a heavy chain Fc domain from an IgG, and a fifth antigen binding domain (E); and the fourth subunit, comprising from the N-terminus to the C-terminus, a sixth antigen binding domain (F) and a CH1 heavy chain constant region; wherein high-affinity binding between the CL light chain constant region in the first subunit and the CH1 heavy chain constant region in the second subunit and a second high-affinity binding between the CL light chain constant region in the third subunit and the CH1 heavy chain constant region in the fourth subunit drive heterodimerization of the molecule.

Embodiment 2. The antibody-derived recombinant antigen binding molecule of embodiment 1, wherein the scaffold comprises a first subunit having an N-terminus and a C-terminus, a second subunit having an N-terminus and a C-terminus, and a third subunit having an N-terminus and a C-terminus; wherein the first subunit, comprising from the N-terminus to the C-terminus, a first antigen binding domain (A) and a CL light chain constant region; the second subunit, comprising from the N-terminus to the C-terminus, a second antigen binding domain (B), a CH1 heavy chain constant region, an immunoglobulin hinge region, a heavy chain Fc domain from an IgG, and a third antigen binding domain (C); and the third subunit, comprising from the N-terminus to the C-terminus, a fourth antigen binding domain (D), an immunoglobulin hinge region, a heavy chain Fc domain from an IgG, and a fifth antigen binding domain (E); wherein high-affinity binding between the CL light chain constant region in the first subunit and the CH1 heavy chain constant region in the second subunit drive heterodimerization of the molecule.

Embodiment 3. The antibody-derived recombinant antigen binding molecule of embodiments 1 and 2, wherein the scaffold comprises a first subunit having an N-terminus and a C-terminus and a second subunit having an N-terminus and a C-terminus; wherein the first subunit, comprising from the N-terminus to the C-terminus, a first antigen binding domain (A), a CL light chain constant region, an immunoglobulin hinge region, a heavy chain Fc domain from an IgG, and a second antigen binding domain (B); the second subunit, comprising from the N-terminus to the C-terminus, a third antigen binding domain (C), a CH1 heavy chain constant region, an immunoglobulin hinge region, a heavy chain Fc domain from an IgG, and a fourth antigen binding domain (D); and wherein high-affinity binding between the CL light chain constant region in the first subunit and the CH1 heavy chain constant region in the second subunit drive heterodimerization of the molecule.

Embodiment 4. An antibody-derived recombinant antigen binding molecule comprising a scaffold with a first subunit having an N-terminus and a C-terminus and a second subunit having an N-terminus and a C-terminus; and the first subunit, comprising from the N-terminus to the C-terminus, a first antigen binding domain (A) and a CH1 heavy chain constant region; and the second subunit, comprising from the N-terminus to the C-terminus, a second antigen binding domain (B) and a CL light chain constant region of an Igκ or an Igλ; wherein the first subunit and the second subunit are linked to a chimeric antigen receptor, wherein the chimeric antigen receptor comprises a hinge region, a transmembrane domain, and at least one intracellular signaling domain; and wherein the high-affinity binding between the CH1 heavy chain constant region in the first subunit and the CL light chain constant region in the first subunit drive heterodimerization of the molecule.

Embodiment 5. The antibody-derived recombinant antigen binding molecule of embodiment 4, wherein the chimeric antigen receptor is a phagocytic chimeric antigen receptor.

Embodiment 6. An antibody-derived recombinant antigen binding molecule comprising a scaffold with at least a first subunit having an N-terminus and a C-terminus, at least a second subunit having an N-terminus and a C-terminus; and at least one subunit of a tumor necrosis factor superfamily (TNFSF) ligand; the first subunit, comprising from the N-terminus to the C-terminus, a first antigen binding domain, a CH1 heavy chain constant region, an immunoglobulin hinge region, a heavy chain Fc domain from an IgG, and a second antigen binding domain; and the second subunit, comprising from the N-terminus to the C-terminus, a third antigen binding domain, a CL light chain constant region of an Igκ or an Igλ, an immunoglobulin hinge region, a heavy chain Fc domain from an IgG, and a fourth antigen binding domain; and wherein the TNFSF ligand is linked to a terminal end of the first and/or second subunit.

Embodiment 7. A single chain antibody-derived recombinant antigen binding molecule comprising an antigen binding domain and at least one subunit of a tumor necrosis factor superfamily (TNFSF) ligand, wherein at least one subunit of the TNFSF ligand is tethered by a flexible linker to an N-terminal and/or a C-terminal antigen binding domain.

Embodiment 8. The antibody-derived recombinant antigen binding molecule of any one of embodiments 1-6, wherein the CH1 heavy chain constant region is selected from the group of an IgM, an IgA, an IgG₂, or an IgG₃.

Embodiment 9. The antibody-derived recombinant antigen binding molecule of any one of embodiments 1-7, wherein the antigen binding domain is a Fv, a F(ab′)₂, a Fab, a scFv, a VHH based single antigen binding domain, or natural protein domains.

Embodiment 10. The antibody-derived recombinant antigen binding molecule of embodiment 9, wherein the natural protein domains include immune checkpoint proteins, cytokines, or natural killer receptors.

Embodiment 11. The antibody-derived recombinant antigen binding molecule of embodiment 10, wherein the immune checkpoint protein is PD-1.

Embodiment 12. The antibody-derived recombinant antigen binding molecule of embodiment 10, wherein the cytokine is IL15 or IL15-alpha.

Embodiment 13. The antibody-derived recombinant antigen binding molecule of embodiment 10, wherein the natural killer receptor is NKG2D, NKp44, NKp46, NKp30, or NKRP1A.

Embodiment 14. The antibody-derived recombinant antigen binding molecule of any one of embodiments 1-6, wherein the N-terminus of at least one antigen binding domain is linked to an antigen binding molecule, wherein an immunoglobulin hinge region links the N-terminus of the antigen binding domain to the antigen binding molecule.

Embodiment 15. The antibody-derived recombinant antigen binding molecule of embodiment 14, wherein the antigen binding molecule is an IL15 alpha receptor, IL15 receptor, or NKG2D dimer.

Embodiment 16. The antibody-derived recombinant antigen binding molecule of embodiments 14 and 15, wherein the antigen binding molecule is the same at each N-terminal antigen binding domain.

Embodiment 17. The antibody-derived recombinant antigen binding molecule of embodiments 14-16, wherein the antigen binding molecule is different at each N-terminal antigen binding domain.

Embodiment 18. The antibody-derived recombinant antigen binding molecule of any one of embodiments 1-3, wherein the Fc domain comprises either a disulfide bridge or a knob-into-hole modification, or the Fc domain comprises both a disulfide bridge and a knob-into-hole modification.

Embodiment 19. The antibody-derived recombinant antigen binding molecule of any one of embodiments 1-3, wherein the Fc domain comprises a CH2 domain of an IgG and a CH3 domain of an IgG.

Embodiment 20. The antibody-derived recombinant antigen binding molecule of embodiment 3, wherein the Fc domain comprises a CH2 domain of an IgG, a first CH3 domain of an IgG, and a second CH3 domain of an IgG.

Embodiment 21. The antibody-derived recombinant antigen binding molecule of embodiments 18-20, wherein the C-terminus of at least one CH3 domain is linked to an antigen binding molecule.

Embodiment 22. The antibody-derived recombinant antigen binding molecule of embodiments 14-16 and, wherein the antigen binding molecule is PD-1.

Embodiment 23. The antibody-derived recombinant antigen binding molecule of any one of embodiments 1-22, wherein at least one antigen binding domain is specific for binding to a tumor associated antigen, a tumor specific antigen, a leukocyte antigen, an effector cell receptor, a cytokine receptor, a Toll-like receptor (TLR), a phagocytic receptor, or an immune checkpoint protein.

Embodiment 24. The antibody-derived recombinant antigen binding molecule of embodiment 23, wherein the tumor associated antigen is Trop2, Her2, CD19, CD22, MR1, CD1, MICA/B, ULBP, CLEC2D, or NKp44L.

Embodiment 25. The antibody-derived recombinant antigen binding molecule of embodiment 23, wherein the effector cell receptor is TCRs, NK receptors, DR5, phagocytic receptors, or TLRs.

Embodiment 26. The antibody-derived recombinant antigen binding molecule of embodiment 23, wherein the cytokine receptor is IL15, IL12, or IL7.

Embodiment 27. The antibody-derived recombinant antigen binding molecule of embodiment 23, wherein the immune checkpoint protein is PD-1, PD-L1, TIGIT, or CTLA-4.

Embodiment 28. The antibody-derived recombinant antigen binding molecule of any one of embodiments 1-27, wherein the heterodimerization is further stabilized by modifying the interaction between the CL light chain constant region and the CH1 heavy chain constant region.

Embodiment 29. The antibody-derived recombinant antigen binding molecule of any one of embodiments 1-28, wherein the heterodimerization is further stabilized by the addition of a disulfide bridge to the Fc domain.

Embodiment 30. The antibody-derived recombinant antigen binding molecule of any one of embodiments 1-29, wherein the heterodimerization is further stabilized by the addition of a knob-into-hole modification.

Embodiment 31. The antibody-derived recombinant antigen binding molecule of any one of embodiments 1-30, wherein the immunoglobulin hinge region is 3-9 amino acids in length.

Embodiment 32. The antibody-derived recombinant antigen binding molecule of embodiment 31, wherein the immunoglobulin hinge region comprises glycine amino acids.

Embodiment 33. The antibody-derived recombinant antigen binding molecule of embodiments 32 and 33, wherein the immunoglobulin hinge region comprises proline amino acids.

Embodiment 34. The antibody-derived recombinant antigen binding molecule of embodiments 4 or 5, wherein the transmembrane domain is from a CD 28 T cell.

Embodiment 35. The antibody-derived recombinant antigen binding molecule of embodiments 4 or 5, wherein the transmembrane domain is from a CD 28 T cell.

Embodiment 36. The antibody-derived recombinant antigen binding molecule of embodiment 4, wherein the intracellular signaling domain is selected from the group of CD137, CD3ζ, CD40, ICOS, CD2, DAP10, and DAP12.

Embodiment 37. The antibody-derived recombinant antigen binding molecule of embodiment 5, wherein the intracellular signaling domain is selected from the group of SIRPa, CR1, CD11b+CD18, aV+b3/b5, MER tyrosine kinase, AXL tyrosine kinase, and scavenger Rcs.

Embodiment 38. The antibody-derived recombinant antigen binding molecule of embodiments 6 or 7 comprising one subunit of the TNFSF ligand.

Embodiment 39. The antibody-derived recombinant antigen binding molecule of embodiments 6 or 7 comprising two subunits of the TNFSF ligand.

Embodiment 40. The antibody-derived recombinant antigen binding molecule of embodiments 6, 7, or 38-40 comprising an antigen binding domain selected from the group of Fab, Fv, scFv, scFv and a receptor, a receptor, and two or more receptors.

Embodiment 41. A genetically modified host cell that can produce an antibody-derived recombinant antigen binding molecule according to any one of embodiments 1-40, wherein the genetic modification comprises a recombinant expression vector, and wherein the genetically modified host cell produces an increased amount of the antibody-derived recombinant antigen binding molecule compared to host cells which are not genetically modified in the same way.

Embodiment 42. The genetically modified host cell of embodiment 41, wherein the recombinant expression vector comprises at least one heterologous nucleic acid encoding at least one subunit.

Embodiment 43. The genetically modified host cell of embodiments 41 and 42, wherein the recombinant expression vector is a plasmid or a virus.

Embodiment 44. The genetically modified host cell of embodiments 41-43, wherein the genetically modified host cell is a mammalian cell.

Embodiment 45. The genetically modified host cell of embodiments 41-44, wherein the mammalian cell is a HEK293 cell, a COS cell, or a CHO cell.

Embodiment 46. A method to express an antibody-derived recombinant antigen binding molecule, the method comprising: culturing a genetically modified host cell comprising at least one recombinant expression vector that can express the antibody-derived recombinant antigen binding molecule according to any one of embodiments 1-40; wherein culturing the genetically modified host cell under conditions such that high-affinity binding between the CL light chain constant region and the CH1 heavy chain constant region drive heterodimerization of the molecule; and purifying the heterodimeric recombinant antigen binding molecule from the culture media.

Embodiment 47. The method of embodiment 46, wherein the recombinant expression vector comprises at least one heterologous nucleic acid encoding at least one subunit.

Embodiment 48. The method of embodiments 46 and 47, wherein the recombinant expression vector is a plasmid or a virus.

Embodiment 49. The method of embodiments 46-48, wherein the genetically modified host cell is a mammalian cell.

Embodiment 50. The method of embodiments 46-49, wherein the mammalian cell is a HEK293 cell, a COS cell, or a CHO cell.

In this context, the following examples illustrate that various antibody-based recombinant antigen binding molecule formats can be designed for safety, efficient manufacturing, stability, and multiple anti-tumor activities contributing to greater efficacy.

### EXAMPLES

The present disclosure is further described with reference to the following examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teaching provided herein.

### Example 1—Production of 2-Subunit TetrAx Molecules

In this example, a TetrAx antibody-derived antigen binding molecule comprised of two, three or four subunits, can be expressed using up to four expression constructs that each encode one or two subunits. Examples of these subunits and their primary sequence are described in FIG. 2 and in SEQ ID NOs: 1-32 and 56-78 (Tables 1 and 7). The expression construct, from the 5′ end to the 3′ end can comprise a CMV or EF1a promoter, a consensus Kozak translational start site, a coding region, a stabilized 3′UTR and a polyadenylation site. The coding region codons for each subunit are optimized for the species of cells used for production. For the generation of stable production lines, the expression plasmids can comprise a drug resistance gene for puromycin, hygromycin or geneticin and/or glutamine synthetase (GS)—for production in GS null cells. For production in CHO cells, the promoter can be a Chinese hamster elongation factor (EF-1a) or CHEF1 (Running Deer J, Allison D S. High-level expression of proteins in mammalian cells using transcription regulatory sequences from the Chinese hamster EF-1alpha gene. Biotechnol Prog. 2004 May-June; 20(3):880-9. doi: 10.1021/bp034383r. PMID: 15176895).

The expression plasmids are introduced into mammalian host cells, such as HEK293 cells or CHO cells by standard transfection methods such as PEI, lipofectamine, or electroporation. Co-transfection can be conducted at different expression plasmid ratios to optimize production. After appropriate culture duration, and drug selection (for generation of stable producer lines), the TetrAx antibody-derived antigen binding molecules can be purified from filtered culture media using standard methods such as Protein-A FPLC and ion-exchange chromatography.

To determine efficiency of TetrAx heterodimerization, a TetrAx core with an N-terminal Fv was produced. An expression construct encoding a light chain (containing CL) comprising a VK, CL and G₁ Fc (hole), and an expression construct encoding a heavy chain (containing CH1), comprising a VH, MCH1 and G₁ Fc (knob), were co-transfected into HEK293 cells. The plasmids were transfected at a 2:1, 1.5:1, and 1:1 light to heavy (L:H) chain ratios. A second pair of expression constructs were co-transfected that were identical except that the MCH1 encoding sequence was replaced with the CH1 from IgA. At the same time identical expression constructs encoding the light and heavy chains of a therapeutic antibody, Nivolumab, were co-transfected at a typical 1:1 ratio. After 5 days in culture, media was harvested, TetrAx and antibody concentrations were determined by bio-layer interferometry (BLI) (FIG. 3A). The greatest concentration of TetrAx (at 2:1) was almost 2-fold greater than that of the protein that contained the IgA CH1 (at 1:1). The TetrAx concentration was approximately 4-fold greater than that of Nivolumab. Bivalent TetrAx were subsequently produced at similarly high levels.

The production of three TetAx that differed only by CH1 isotype; MCH1, A1CH1 or G1CH1 were compared (FIG. 2B). These were expressed in HEK293 cells as described above. They were purified by protein-A FPLC and quantitated by BLI. The TetrAx possessing the MCH1 demonstrated the greatest production whereas that possessing A1CH1 was 40% lower and that possessing G1CH1 was 60% lower. The results indicate that the production of certain TetrAx can be advantaged with MCH1.

A series of TetrAx light (17) and heavy (18) chains were expressed in HEK293 cells and visualized by SDS-PAGE (FIG. 3). Culture media containing the various TetrAx molecules were loaded on to a non-reducing SDS-PAGE (after concentrations were adjusted to load approximately 2 μg/lane), electrophoresed and stained (FIG. 3A). All TetrAx molecules comprised the core with an N-terminal Fv. These multispecific TetrAx molecules possessed various binding domains including PD1 ectodomain, b2GP1-D5, and aCD137L monomer. All TetrAx demonstrated a single major product relative to mock transfected culture media. Homodimeric light chains (with hole Fc) were not evident. These results indicate that TetrAx cores can support efficient heterodimerization.

TetrAx molecules were purified in a single step by protein-A FPLC and subjected to SDS-PAGE under non-reducing (FIG. 3B) and reducing conditions (FIG. 3C). The purified TetrAx molecules resolved as a single prominent apparent molecular weight and appeared to be ≥90% pure. Under reducing conditions, the apparent molecular weights for the different subunits are consistent with their calculated weights. Transfection of light chains (with hole Fc) alone does not result in the production of homodimers, indicating that the position of CL in TetrAx can inhibit homodimerization.

The effect of the TetrAx MCH1 N-linked glycosylation on binding to a target effector cell receptor (CD3) was determined (FIG. 4). A TetrAx core with an N-terminal Fv specific for CD3 was used to determine the effect of glycosylation at the MCH1 NNS site. This MCH1 N-linked glycosylation site is located in a loop proximal to the N-terminal Fv (FIG. 1) and its oligosaccharide could sterically interfere with the Fv binding to a cellular target. The NXT/S N-linked glycosylation motif in NNS was eliminated by amino acid substitution mutation (NNS/NNA). The NNS/NNA mutation resulted in an appropriate reduction in apparent molecular weight as determined by SDS-PAGE (FIG. 4A, lane2). There was no further reduction in size with a NPS/SPS mutation indicating that, NPS, as expected does not appear to be N-linked glycosylated (FIG. 4A, lane 3).

TetrAx molecules, with and without N-linked site mutations, were produced in HEK293 cells and binding to T cells was determined by flow cytofluorimetry. The binding of the TetrAx molecule with the NNS/NNA substitution (FIG. 4C) was not significantly different from that with intact N-linked sites (FIG. 4B), at 1, 5 and 15 μg/ml.

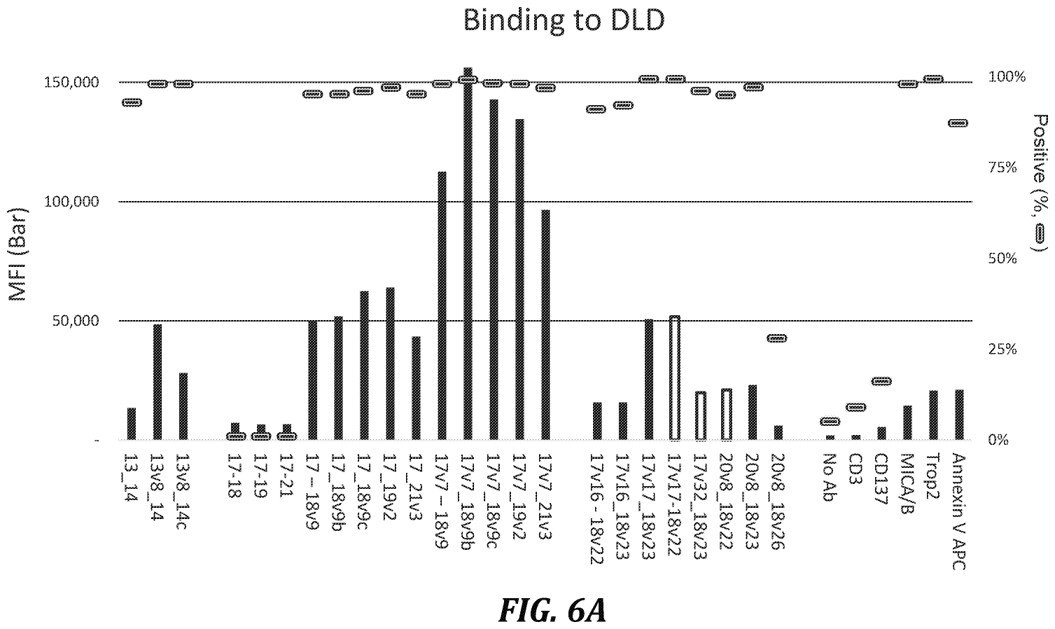

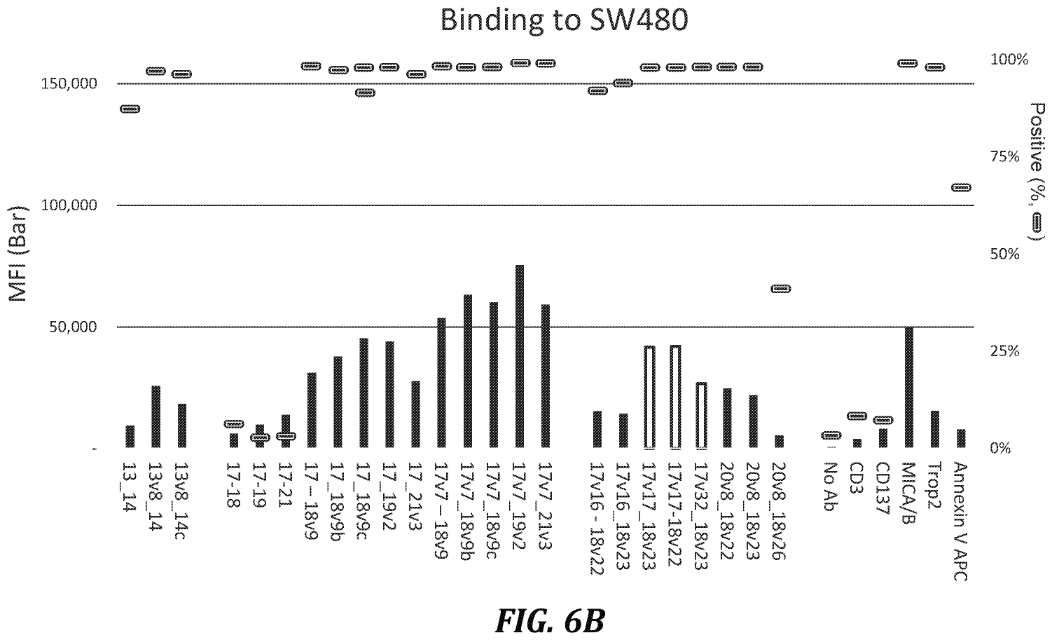

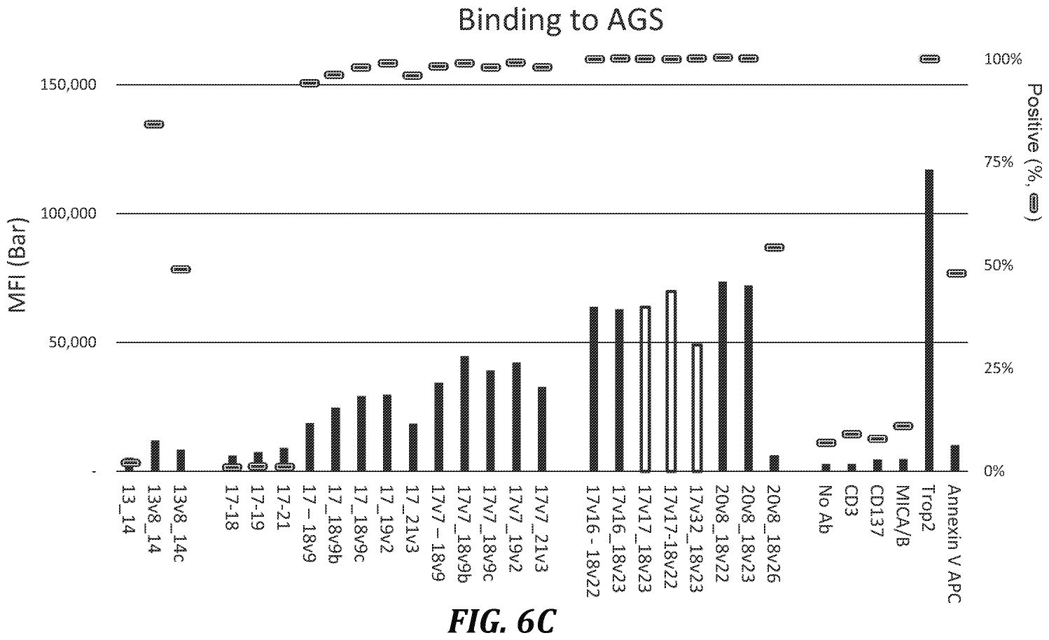

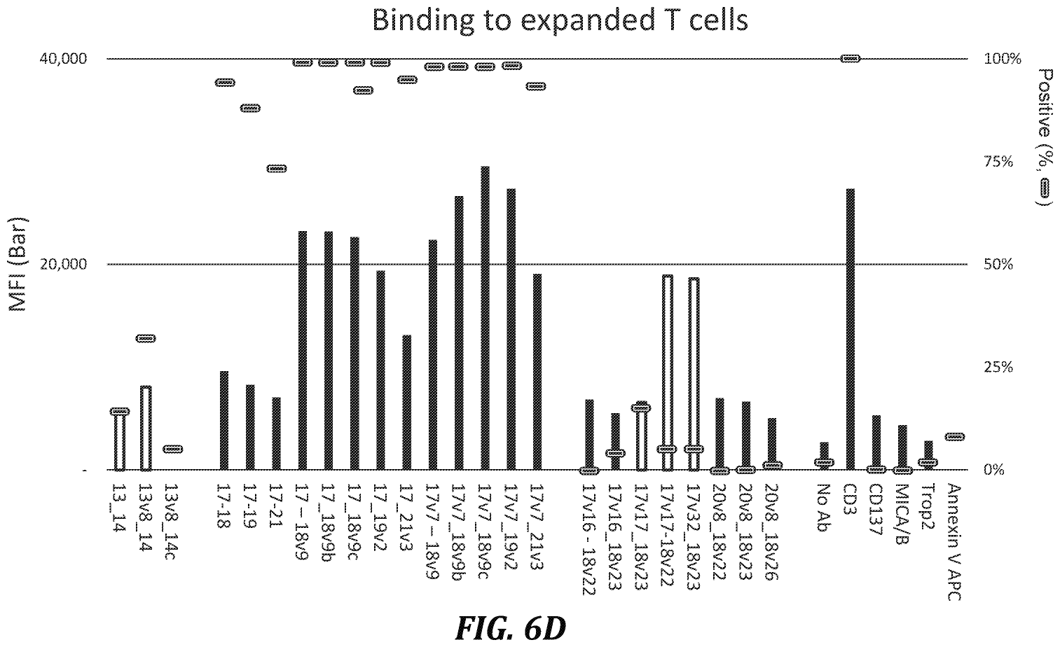

Comparison of mean fluorescence intensity (MFI) indicated a slight increase of the TetrAx with NNS/NNA at high concentration (15 μg/ml, FIG. 4E). Thus, for this TetrAx molecule and the target antigen, it does not appear that the MCH1 N-linked glycosylation site interferes with binding to cell target antigens. Similarly, there was no significant difference in T cell binding of a pair of MCH1 possessing TetrAx (17v7-18v9b and 17v7-18v9c) that were identical expect for the NNS/NNA mutation (17v7-18v9c) disrupting the sole MCH1 N-linked glycosylation site (FIG. 6D).

The binding of the TetrAx molecule comprising one or two β2GP1 (domain 5) phosphatidylserine binding domains was determined as illustrated in FIG. 5. In FIG. 5, monovalent TetrAx molecules were constructed with domain 5 of b2GP1 linked to the C-terminus of the light chain (17v7) or the heavy chain (18v9). Both chains were co-transfected to generate bivalent TetrAx molecules. These TetrAx molecules were expressed at levels greater than that of a therapeutic monoclonal antibody in the same system indicating that bivalent and trivalent TetrAx can be expressed at high levels for efficient manufacturing.

Phosphatidylserine is exposed on the surface of many tumor cells and on stressed cells. TetrAx (17,18v9) bound to a viable population of HEK293 cells (propidium iodine negative; FIGS. 5A and 5B). Binding to HEK293 cells increases when they are stressed by culturing to high density for 5 days without addition of fresh culture media (FIG. 5C). Increased binding to stressed cells is also demonstrated for the positive control Annexin V (FIG. 5D). As expected, the binding of bivalent TetrAx to the colon cancer cell line (DLD1) is greater compared to the monovalent TetrAx molecule (FIG. 5E).

Specific binding to purified phosphatidylserine coated wells was demonstrated by ELISA. TetrAx specifically bound to phosphatidylserine, relative to TetrAx lacking the b2GP1 domains (FIG. 5F). Binding was apparently lower affinity relative to the high affinity annexin V binding. These data indicate that functional multivalent/multispecific 2-subunit TetrAx molecules can be efficiently produced.

The binding of a panel of multispecific TetrAx to tumor target cells and T effector cells was compared by flow cytofluorimetry (FIG. 6). The pattern of binding for TROP2 and PS specific TetrAx and commercial antibodies specific for TROP2 and PS (FIG. 6A-6B; right most bars) indicates that DLD1 express relatively high levels of TROP2 and low level of PS (6A). SW480 cells express relatively moderate to low TROP2 and low levels of PS (6B) and AGS cells express relatively low levels of TROP2 and moderate levels of PS (6C). Different TetrAx varied some in their MFI but were more consistent in percent binding.

### Example 2—Production of 3- and 4-Subunit TetrAx Molecules

In this example, three and four subunit TetrAx molecules can be produced as described previously for two subunit TetrAx molecules. See Example 1. Examples of three and four subunit TetrAx confirmations are described in Table 1 and their primary structures are illustrated in FIG. 2 and in SEQ ID NOs: 13-26 (Table 7). Each subunit can be expressed from a separate expression vector as described for the 2 subunit TetrAx molecule in Example 1. Typically, each expression construct, from the 5′ end to the 3′ end will comprise a CMV promoter or an EFa promoter, a strong Kozak translational start site, a coding region, a stabilized 3′UTR (see Example 5) and a polyadenylation site.

A three subunit TetrAx molecule (30v1-24-39) was produced with a light chain containing a VK and CL; a heavy chain comprised of a VH, MCH1, G₁ Fc (knob); and a third subunit comprised of the N-terminal domain of TIM4, a linker, the N-terminal domain of TIM1, and G₁ Fc (knob). A second three subunit TetrAx molecule (30v1-34-39v2) was produced that differed in the third subunit which is comprised of the N-terminal domain of TIM3, linker-hinge, CH2-CH3 (knob). These three subunit TetrAx molecules bind to their target antigen on T cells as determined by flow cytofluorimetry. These data indicate that functional multivalent/multispecific 3- and 4-subunit TetrAx molecules can be efficiently produced.

### Example 3—Production of CHAx Molecules

The CHAx molecule can be produced as described for the two subunit TetrAx molecules. See Example 1. Examples of CHAx molecule confirmations are described in Table 7 (SEQ ID NOs: 27-32). Each subunit can be expressed from a separate expression vector as described previously for the 2-subunit TetrAx molecule. See Example 1. Typically, each expression construct, from the 5′ end to the 3′ end will comprise a CMV promoter or an EFa promoter, a strong Kozak translational start site, a coding region, a stabilized 3′UTR and a polyadenylation site.

For the CHAx molecule (SEQ ID NOs: 29 and 30), the first subunit is comprised of an N-terminal NKG2D domain, a linker, a CH3 (hole), a linker, CH2, and CH3 (knob). The second subunit is comprised of an N-terminal anti-Trop2scFv, a linker, an NKG2D domain, a linker, CH3 (knob) linker, CH2 and CH3 (hole). Binding to target T cells was verified by flow cytofluorometry. These data indicate that functional novel CHAx molecules can be produced.

### Example 4—Production of MCAR/MPCAR Molecules

The heterodimeric structure of MCAR and MPCAR cores is advantageous relative to typical CARs because the MCAR and MPCAR cores provide a greater capacity for attaching additional binding and signaling domains to enable additional mechanisms of activity and potentially greater efficacy. Additionally, the high affinity MCH1-CL interaction supports heterodimerization, serves as a platform for various binding domains and brings co-operating signaling domains into close proximity.

In this example, the expression constructs for the two subunits of MCAR or MPCAR can be generated in the same fashion as described previously for the 2-subunit TetrAx molecule. See Example 1. However, the coding region for the MCAR and MPCAR is different from the TetrAx molecules. For example, the coding region comprises, from the 5′ end to the 3′ end, a signal sequence, a binding domain, a CH1 or CL, a stalk, a transmembrane domain, and endodomains that enable signaling and/or phagocytosis. Table 2 (SEQ ID NOs: 33-50).

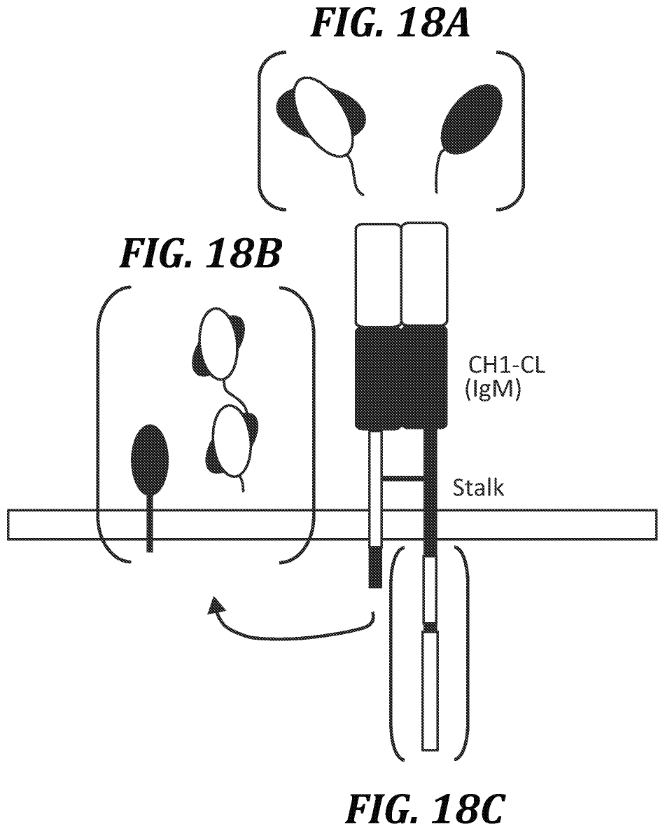

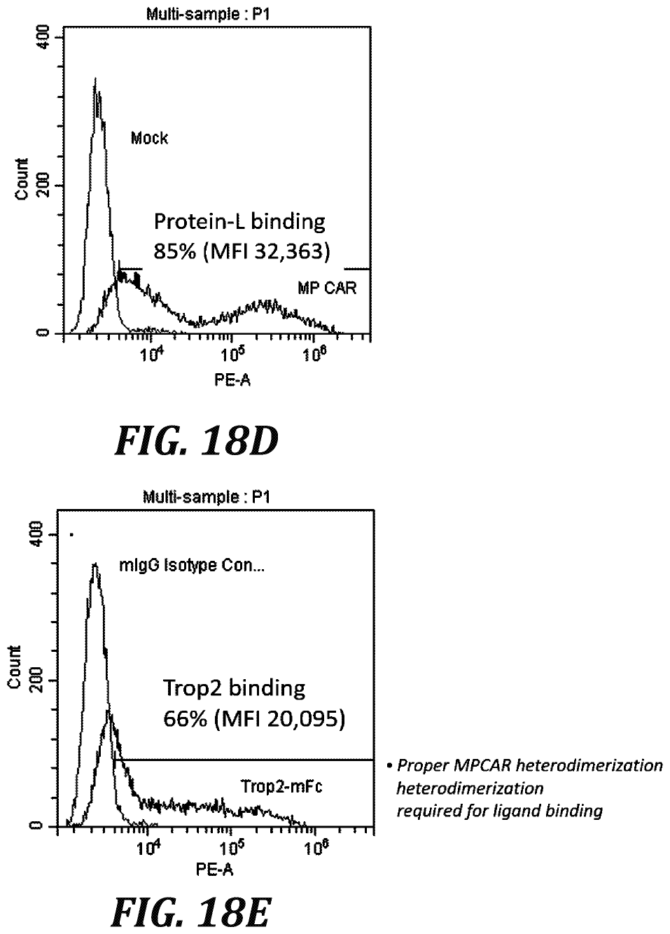

To express an MPCAR molecule capable of binding to TROP2, two vectors were constructed. The first vector encodes the first monomer comprising a VK, CL, CD8 stalk, a transmembrane region, and a CD11b, CD3z endodomain (SEQ ID NO: 43). The second vector encodes the second monomer comprising a VH, MCH1, CD8 stalk, a transmembrane domain, and a CD18, CD137 endodomain (SEQ ID NO: 44). The expression vectors were co-transfected into HEK293 cells using standard polyethyleneimine (PEI) mediated transfection. MPCAR expression was determined two days post-transfection by flow cytofluorimetry using Protein-L biotin, which binds to the MPCAR VK, and the detection reagent, streptavidin-PE. Using this method, approximately, 85% of the transfected cells expressed MPCAR. Expression level in transfected cells was typically broad, with a mean florescence intensity (MFI) of 32,363 counts as reported in FIG. 18D.

MPCAR binding to target antigen was determined by flow cytofluorimetry using a soluble Trop2-Fc fusion protein. The transfected cells were stained with 5 μg/ml of Trop2-Fc and binding was detected with and anti-mouse Fc-PE conjugate. Trop2 bound to MPCAR over a broad range with an MFI of 20,095 counts (FIG. 18E). Neither Protein-L nor Trop2 bound to mock transfected HEK293 cells. Trop2 binding would require correct heterodimerization of the two MPCAR subunits to form a functional Fv specific for TROP2. MCH1-CL therefore efficiently supports functional MPCAR heterodimerization and expression. Thus, these data demonstrate that novel functional heterodimeric MPCAR molecules can be produced.

### Example 5—pR Construct for In Vitro Synthesis of Stabilized MCAR or MPCAR mRNA

In this example, to synthesize MCAR or MPCAR (or TetrAx, or CHAx) mRNA, a vector is constructed for in vitro transcription (IVT). The vector (pR) comprises a small 1.6 kb backbone with Kanamycin resistance and a T7 promoter. An mRNA can be synthesized using pR and a standard IVT kit. Prior to synthesis, pR is linearized at the 3′ Spe1 site. An mRNA is synthesized that comprises a Kozak translational start, a coding region, a 1^{st} 3′UTR, a 2^{nd} 3′UTR, in some instances a 3^{rd} 3′UTR, and a poly-adenosine sequence of over 120 bp.

Typically, the 3′UTR originates from β-globin, however, other mRNA stabilizing 3′UTRs have been identified (Holtkamp S, et al., Blood. 2006 Dec. 15; 108(13):4009-17; Orlandini von Niessen A G, et al., Mol Ther. 2019 Apr. 10; 27(4):824-836). The 3′UTR can comprise complementary miRNA target sites where miRNA can bind and repress translation or recruit ribonuclease that results in RNA decay. Novel pR vectors described here contain two or three different sequential modified 3′UTR with certain miRNA target sites eliminated to enhance mRNA stability (Table 5). MiRNA target sequences in the mRNA are identified using one or more publicly available miRNA web tools. The 3′UTR with highly homologous miRNA target sequences are mutated to eliminate functional miRNA binding. For example, one or two nucleotides of the β-globin mi-R-4520 target sequence can be substituted with a different nucleotide to eliminate a functional complementary binding site. MiRNA sites are eliminated that are not predicted to disrupt stabilizing secondary structure and accessibility to stabilizing mRNA binding proteins (Boo S H, Kim Y K. The emerging role of RNA modifications in the regulation of mRNA stability. Exp Mol Med. 2020 March; 52(3):400-408. doi: 10.1038/s12276-020-0407-z. Epub 2020 Mar. 24. PMID: 32210357; PMCID: PMC7156397). For example, the 3′UTR in β-globin comprises target sequences are highly complementary to miR-361 and miR-5009. These mRNA sequences can hybridize to each other as indicated in the predicted secondary structure. In the predicted secondary structure, a Cysteine (C) in the miR5009 binding site forms bonds with a Guanidine (G) in the miR361 binding site. The substitution of a C to a G in the miR5009 site and substitution of a G to a C in the miR361 site can disrupt binding of both miRNA but maintain mRNA secondary structure and its contribution to mRNA stabilization. Enhanced stabilized expression can be determined by magnitude and/or duration of expression of the MCAR or MPCAR molecule in target cells (or concentration of secreted TetrAx or Chax). For example, prolonged expression of the MCAR molecule in T cells or NK cells following electroporation of mRNA with mutated miRNA binding sites, relative to mRNA with intact miRNA target sequences, can demonstrate the benefit of eliminating miRNA sites. mRNA can also be synthesized using pR and an additional polyadenylation step to extend the poly-A tail beyond 120 bp, or with modified bases, to increase stability (Boo S H, et al., Exp Mol Med. 2020 March; 52(3):400-408).

Alternatively, MCAR, MPCAR, TetrAx, or CHAx molecules can be constitutively expressed from a vector comprising a 3′UTR with disrupted miRNA binding sites to enhance expression as shown below in Table 5. The expression vector can comprise a stable promoter such as a CMVa promoter, an EFa promoter, or a CHEF promoter. The vector can also include a translational start site, a signal sequence, a mature protein sequence, a stop codon, 1 to 3 3′UTRs with disrupted miRNA binding sites, and a polyadenylation site. The vector can be integrated into mammalian cells for stable expression.

TABLE 5
lists examples of 3′ UTR substitution mutants to eliminate miRNA binding sites
(one or two nucleotide substitutions generated per miRNA binding site).

| 3′ UTR & | Nucleotide substitution sites (X, Y, or Z) to eliminate | SEQ ID |
|---|---|---|
| miRNA | miRNA binding | NO: |
| ϑ-globin | CTTTCTTGCTGTCCAATTTCTATTAAAGGTTCCTTTGTTCCGTAAG(Z)TC(Z)C(Z) | 112 |
| miR-361 (X) | AACTACTAAAC(X)TG(X)G(X)G(X)G(X)G(X)ATATTATGAAGGGCCTTGAGCAT | |
| miR-106a(Y) | CTGGATTCTGCCTAATAAAAAACATTTATTTTC(Y)ATTG(Y)C(Y)AAT | |
| miR-5009(Z) | | |
| LSP1 | TTCCAGCCAGACACCCGCCCCCCGGCCCTGGCTAAGAAGTTGCTTCCTGTTG(X) | 113 |
| miR-9851 (X) | C(X)C(X)AG(X)C(X)ATGACCTACCCTCGCCTCTTTGATGCCATCCGCTGCCACCT | |
| | CCTTTTGCTCCTGGACCCTTTAGCCTCTCTGCCCTTCCACTCTCTG | |
| AES | CTGGTACTGCATGCACGCAATGCTAGCTGCCCCTTTCCCGTCCTGGGCACCCCG | 114 |
| miR-4481 | AGTCTCCCCCGACCTCGGGTCCCAGGTATGCTCCCACCTCCACCTGCC(X)C(X)C | |
| | (X)AC(X)TC(X)ACCACCTCTGCTAGTTCCAGACACCTCC | |
| mtRNR1 | CAAGCACGCAGCAATG(Y)C(Y)AG(Y)C(Y)TC(Y)AAAACGCTTAGCCTAGCCAC | 115 |
| miR-137(X), | ACCCCCACGGGAAACAGCAGTGATTAACCTTTAG(X)C(X)A(X)A(X)TAAACGAA | |
| miR-558(Y) | AGTTTAACTAAGCTATACTAACCCCAGGGTTGGTCAATTTCGTGCCAGCCAC | |
| CCL22 | GCCTTGGCTCCTCCAGGAAGGCTCAGGAGCCCTACCTCCCTGCCATTATAGCT | 116 |
| miR-4683(X), | GCTCCCCGCCAGAAGCCTGTGCCAACTCTCTGCATTCCCTGATCTCCATCCCTG | |
| miR-1207 and | TGGCTGTCACCCTTGGTCACCTCCGTGCTGTCACTGCCATCTCCCCCC | |
| miR-4763 (Y) | | |

### Example 6—Production and Characterization of Targeting TetrAx Molecules that Localize Activity to a Tumor Microenvironment

### Formats of Targeted TNFSF Ligands.

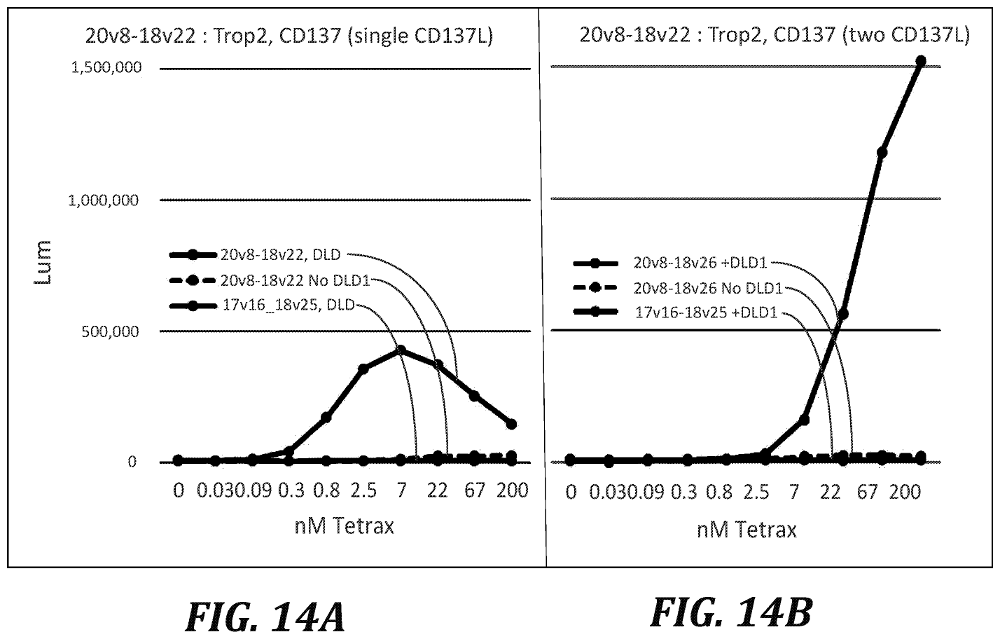

Several distinct structures have been generated to target TNFSF ligands and T cell engagers to universal tumor antigens (FIG. 2 and Table 6). These include TetrAx (A-B; SEQ ID NOs: 56-68), modified Fc (C; SEQID 69-74, 79), whole antibody (B; SEQIDs 75-77), antibody fragments (E-H; SEQIDs 80-85) and small native receptor targeted TNFSF ligands or StaT (F-I; SEQID 86, 87). The native TNFSF ligand is a trimer of identical subunits, however, it was demonstrated that TetrAx with one or two CD137L domains can support signaling (FIG. 14). As illustrated (FIG. 2), the multispecific recombinant antigen binding molecules can possess a single TNFSF ligand domain, or two-three domains. The TNFSF ligand domain(s) can be linked to the C-terminus or N-terminus. The two TNFSF ligand domains can be connected to each other with a linker from the C-terminus of the first to the N-terminus of the second to form a single chain (sc) TNFSF ligand (for example SEQ ID NO: 87). A molecule can possess more than one ligand domain such as CD137L and CD40L, each linked to one heavy chain of a knob-into-hole antibody. Smaller targeted TNFSF (STaT) molecules can be generated that lack an Fc. The smallest molecule depicted here, is approximately 47 kDa. Smaller molecules can result in greater tumor penetration and small single chain molecules can facilitate manufacturing. STaT molecules can induce an anti-tumor immune response over several administrations and efficacy is not dependent on a long circulatory half-life.

TABLE 6
Examples of TNFSF ligands and T cell engagers targeted to universal
and select antigens

| | | | SEQ |
|---|---|---|---|
| Format | | Chain designation and structural | ID |
| (FIG. 2) | Specificity | features from N- to C-terminus | NO: |
| 2 chain dimer | Trop2 × | L: 20v8: hTVkb-CL-truncated(t) Ig | 56 |
| | CD137 | hinge-CH2—CH3-linker-CD137L | |
| | | domain | |
| | | H: 18v22: hTVhb-MCH1-linker-tIg | 57 |
| | | hinge-CH2—CH3 | |
| 2 chain dimer | Trop2 × | L: 20v8: hTVkb-CL-linker-tIg | 56 |
| | CD137 | hinge-CH2—CH3-linker-CD137L | |
| | | domain | |
| | | H: 18v26: hTVhb-MCH1-tIg hinge- | 58 |
| | | CH2—CH3-linker-CD137L domain | |
| 2 chain dimer | Trop2 × | L: 17v9: h3Vk-CL-linker-tIg hinge- | 59 |
| | CD3 | G1Fc-linker-hTscFv | |
| | | H: 18: h3Vh-MCH1-linker-tIg | 60 |
| | | hinge-G1Fc | |
| 2 chain dimer | PS × Trop2 × | L: 17v17: hTVkb-CL-linker-tIg | 61 |
| | CD3 | hinge-G1Fc-linker-b2GP1-D5 | |
| | | H: 18v23: hTVhb-MCH1-linker-tIg | 62 |
| | | hinge-G1Fc-linker-h3scFv | |
| 2 chain dimer | PS(×2) × | L: 17v7: h3Vk-CL-linker-tIg hinge- | 63 |
| | CD3 | G1Fc-linker-b2GP1-D5 | |
| | | H: 18v9b: h3Vh-MCH1-linker-tIg | 64 |
| | | hinge-G1Fc-linker-b2GP1-D5 | |
| 2 chain dimer | PS × Trop2 × | L: 20v8: hTVkb-CL-tIg hinge- | 56 |
| | CD137 | CH2—CH3-linker-CD137L domain | |
| | | H: 18v25: hTVhb-MCH1-linker-tIg | 65 |
| | | hinge-G1Fc-linker-b2GP1-D5 | |
| 2 chain dimer | PS × Trop2 × | L: 17v19b: hTVkb-CL-linker-tIg | 66 |
| | TRAIL | hinge-G1Fc-linker-TRAIL domain | |
| | | H: 18v25: hTVhb-MCH1-linker-tIg | 65 |
| | | hinge-G1Fc-linker-b2GP1-D5 | |
| 2 chain dimer | NKG2DL × | L: 17v29: h3Vk-CL-linker-tIg | 67 |
| | CD3 | hinge-G1Fc-linker-NKG2D domain | |
| | | H: 18v42: h3Vh-MCH1-linker-tIg | 68 |
| | | hinge-G1Fc-linker-NKG2D domain | |
| 2 chain dimer | NKG2DL × | 13: NKG2D-linker-tIg hinge-G1Fc | 69 |
| | CD3 | 14: h3scFv-NKG2D-linker-tIg | 70 |
| | | hinge-G1Fc | |
| 2 chain dimer | NKG2DL × | 13v8: NKG2D-linker-tIg hinge- | |
| | CD3 × PS | G1Fc-linker-b2GP1 | |
| | | 14: h3scFv-NKG2D-linker-tIg | 70 |
| | | hinge-G1Fc | |
| single chain | NKG2DL × | NKG2D-linker-G4Fc-linker | 72 |
| dimer | CD137 | CD137L | |
| single chain | PS(×2) × | CD137L-linker-G4Fc-linker-b2GP1 | 73 |
| dimer | CD137 | | |
| single chain | PS(×2) × | TRAIL-linker-TRAIL-linker- | 74 |
| dimer | TRAIL | TRAIL-linker-G4Fc-linker-b2GP1 | |
| 2 chain | Trop2(×2) × | L Chain: h133Vk-CL | 75 |
| tetramer | CD137 | H Chain: h133Vh-CH1-G4Fc- | 76 |
| (Whole | | linker-CD137L domain | |
| antibody | | | |
| format) | | | |
| 2 chain | Trop2(×2) × | L Chain: h133Vk-CL | 75 |
| tetramer | TRAIL | H1 Chain: h133Vh-CH1-Ig hinge- | |
| | | CH2—CH3(knob)-linker-TRAIL- | |
| | | linker-TRAIL | |
| | | H2 Chain: h133Vh-CH1-Ig hinge- | |
| | | CH2—CH3(hole)-linker-TRAIL | |
| Single chain | CD40(×2) × | CD40L-linker-CD40L-linker- | 79 |
| dimer | PS(×2) | CD40L-linker-tIg hinge-G4Fc- | |
| | | linker-b2GP1 | |
| 2 chain | PS(×2) × | L: hTVk-CL-tIg hinge-CD137L | 80 |
| | CD137 | domain | |
| | | H: hTVh-CH1-tIg hinge-CD137L | 81 |
| | | domain | |
| 2 chain | Trop2 × | L: h133Vk-tIg hinge-CD137L | 82 |
| | CD137(×2) | domain | |
| | | H: h133Vh-tIg hinge-CD137L | 83 |
| | | domain | |
| Single chain | Trop2 × | h133Vh-Vk (scFv)-linker-CD137L | 84 |
| | CD137 | domain-Linker-CD137L domain | |
| Single Chain | PS × Trop2 × | h133scFv-linker-CD137L-linker- | 85 |
| | CD137 | CD137L-linker-b2GP1 | |
| Single Chain | PS × CD137 | Tim4-linker-CD137L-linker- | 86 |
| | | CD137L | |
| Single Chain | TRAIL × | TRAIL-linker-TRAIL-linker- | 87 |
| | PS(×2) | TRAIL-linker-b2GP1-linker b2GP1 | |

The TNFSF ligand domains are targeted to tumor antigens via the specificity of their Fab, scFv, Fv or native receptor domains. For example, a receptor-based targeting domain can be the ecto-domain of NKG2D (targeting tumor antigens such as MICA, MICB), or b2GP1 or Tim4, which target a universal tumor target and immune checkpoint, phosphatidylserine (PS) or an scFv targeting the tumor antigen TROP2. Targeting domains are exchangeable and can be specific for one universal tumor antigen, such as PS, FAPa, NKG2DL, and B7H3, or a combination of a universal and tumor associated antigen (TAA) such as (but not limited to) TROP2, CD19, CD20, CD22, Glypican-3, HER2, EGFR1, and Claudin 18.2. Thus, the Fab in FIG. 2D can be replaced with the dimeric ectodomain of NKG2D. As illustrated, the targeting domain can be linked to the N- or C-terminus of the molecule and can be monovalent or bivalent (FIG. 2). Targeting domains can locate effector domains to a tumor microenvironment (TME). Effector domains can support induction of an immune response (CD137L, CD40L, GIRTL, LIGHT, OX40L, CD27L) and/or tumor cytotoxicity (TRAIL, CD40L, CD137L, anti-CD3).

### Multispecific Recombinant Molecules Binding to T Cells and Tumor Target Cells.

The binding of the multispecific recombinant antigen binding molecules to expanded T cells (FIG. 6D) and to the colon tumor cell line DLD1 (FIG. 6A) was determined by flow cytofluorimetry. Binding was conducted with a saturating concentration (250 nM). The T cells were expanded in culture from immunocult (STEMCELL Technologies) stimulated peripheral blood mononuclear cells that were maintained in culture for at least 30 days. Multispecific recombinant antigen binding molecules were produced in HEK293 cells and one-step purified by FPLC using a HiTrap Protein A column. The expression of various T cell markers or tumor antigens was determined using commercial antibodies specific for Trop2, CD137, MICA/MICB, Tim4, Tim1 (all purchased from Biolegend). The multispecific recombinant antigen binding molecules exhibited a range of T cell binding levels (FIG. 6D). The multispecific recombinant antigen binding molecules also exhibited a wide range of binding to DLD1, PS, TROP2, or NKG2D ligands (FIG. 6A). Several that exhibited potent cytotoxicity, for example targeting NKG2D ligands (13-14 and 17v&-18v23) exhibited low MFI and high percent binding, reflecting characteristically low density expression of NKG2D ligands. Greater levels of binding to DLD1 are associated with binding to PS and in particular bivalent binding to PS (e.g., 17v7-18v9).

FIG. 6 illustrates that various trispecific TetrAx can be created that bind to their target cell antigens. For example, the TetrAx platform allows efficient binding to combinations of tumor antigens and effector domains to enhance therapeutic activity for different tumor types through different mechanisms. See e.g., additional Examples as described below.

### Examples of T Cell Activation Properties of Multispecific Recombinant Molecules.

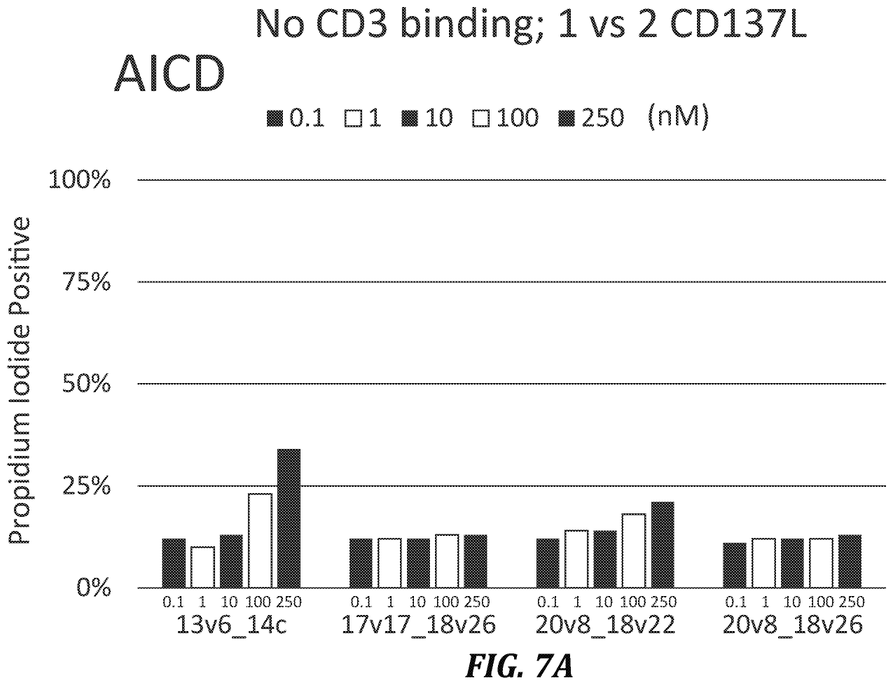

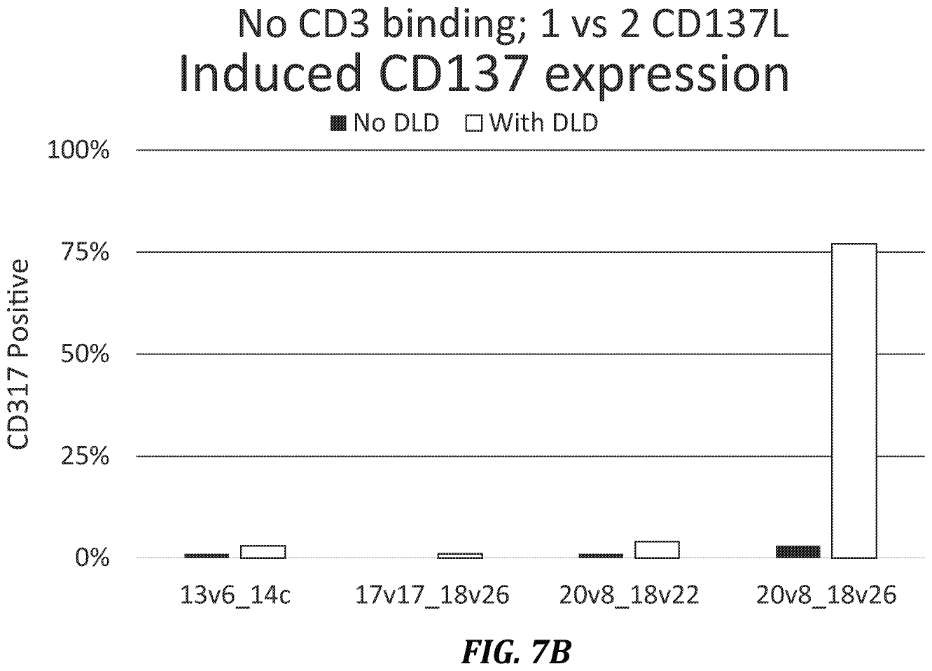

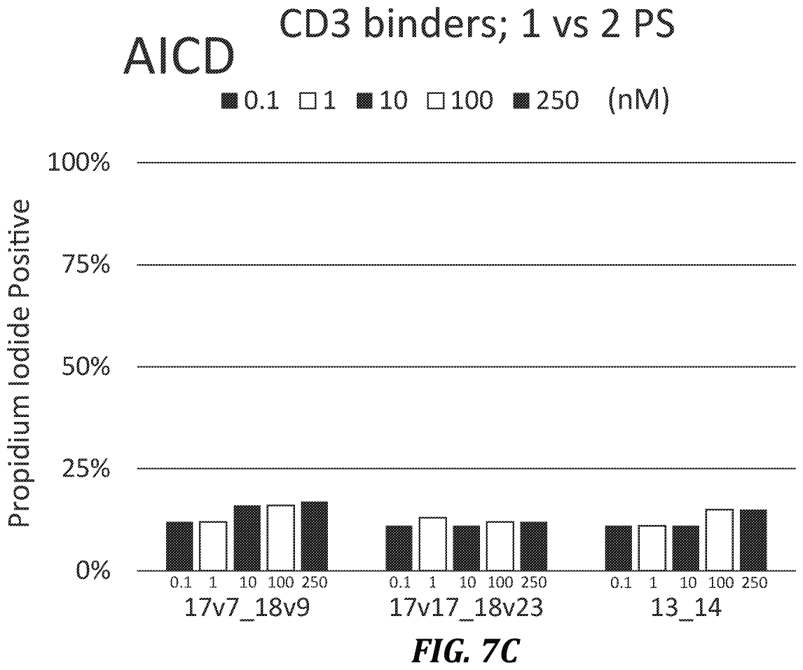

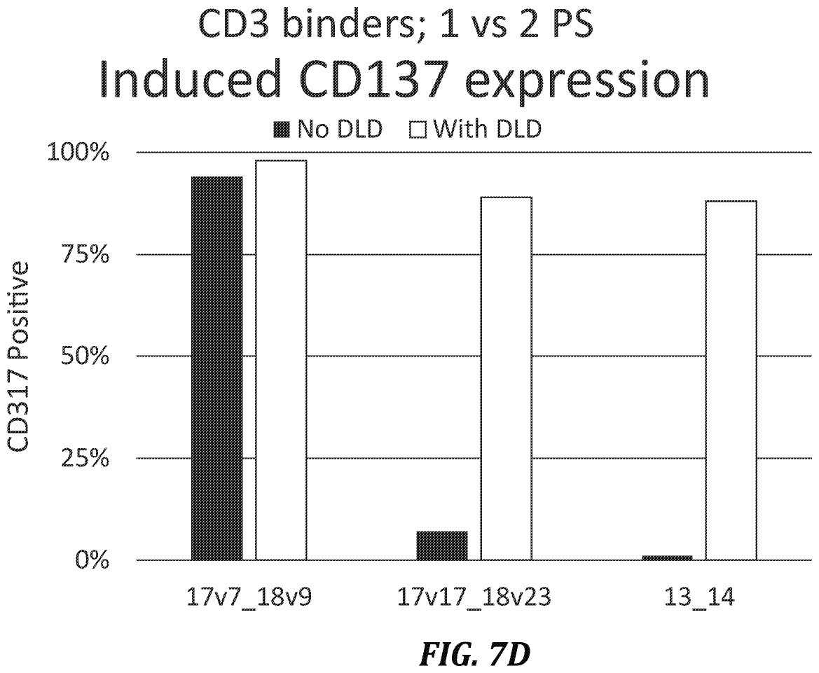

The ability of the multispecific recombinant antigen binding molecules to induce activation induced T cell death (AICD) or upregulation of T cell CD137 expression on expanded T cells, was determined. AICD was determined over a concentration range of 0.1 to 250 nM after 20 hours of incubation under tissue culture conditions. AICD was measured by propidium iodine (PI) staining and flow cytofluorometry. These activities were determined for sets of multispecific recombinant antigen binding molecules, that bind tumor TROP2, PS, or NKG2DL tumor antigens. One set binds CD3 (FIGS. 7C and 7D) whereas the other does not bind CD3 (FIGS. 7A and 7B). Only one TetrAx weakly stimulated AICD at high concentrations (FIG. 7A). Induction of CD137 expression was determined by flow cytofluorimetry after 20 hours of incubation in the presence or absence of the target tumor cell DLD1. All TetrAx that bind CD3 induced CD137 expression (FIG. 7D). One TetrAx that does not bind CD3, induced CD137 expression (20v8-18v26; FIG. 7B). This TetrAx targets TROP2 and possesses two CD137L domains.

The data indicate that most of these T cell engagers, that are monovalent for CD3, do not activate T cells, unless they can bind their target antigen on tumor cells and then presumably present an avid binding surface that can crosslink CD3. Surprisingly, a TetrAx that does not engage CD3, but possesses two CD137L domains (not a full native trimer) is also capable of activating T cells in a tumor antigen dependent manner. This mechanism is examined using a CD137 signaling reporter cell line (FIGS. 14-16). In an Fc receptor null form, these molecules are expected to support localized activity, including cytokine release, in the tumor microenvironment (TME). Multispecific TetrAx that also antagonize certain immune check points, in certain cancer settings would be expected to demonstrate yet greater efficacy in a TME. Thus, CD3 engaging and non-CD3 engaging multispecific recombinant antigen binding molecules are identified here that demonstrate appropriate and efficient tumor target antigen dependent T cell activation. These include TetrAx that bind or do not bind CD3 that are suitable for development. These can be developed primarily as stimulators of an anti-tumor response with or without substantial direct tumor cell cytotoxicity. In clinical studies expression of CD137 on peripheral blood T cells can serve as a clinical biomarker for the activity of certain multispecific recombinant antigen binding molecules that induce CD137 expression.

Potent Tumor Cell Cytotoxicity with T Cell Engaging Bispecific Recombinant Antigen Binding Molecules Targeting NKG2D Ligands.

The tumoricidal activity of T cell engaging (CD3) bispecific recombinant antigen binding molecules targeted to NKG2D ligands was determined in an 18-hour assay with T cells and the target colon cancer cell line DLD1. The expression of NKG2D ligands on DLD1 was demonstrated by flow cytometry using a commercial antibody that binds both MICA and MICB (FIG. 6A). The bispecific recombinant antigen binding molecules include 17v29-18v42, a TetrAx with an N-terminal CD3 binding domain and a C-terminal NKG2D dimer and 13-14, an Fc with an N-terminal NKG2D dimer in which a CD3 binding scFv was linked to one NKG2D domain. These bispecific recombinant antigen binding molecules bound to DLD1 cells and the expanded T cells as determined by flow cytofluorimetry (FIGS. 6A and 6D). For the cytotoxicity assay, DLD1-Luc that constitutively express luciferase, were allowed to adhere to microtiter plate wells overnight to achieve 50-80% confluency and the T cells were then added for a 2:1 effector to target ratio. The bispecific recombinant antigen binding molecules were added to achieve the indicated concentration range. After 18 hours of incubation, the wells were washed, luciferase substrate added and luminescence measured 3 times in a multimode plate reader. EC50 values were determined using PRISM GraphPad software. The data indicates that both bispecific recombinant antigen binding molecules support 100% DLD1 cytotoxicity with similar potency; an EC50 of approximately 0.03 nM. Thus, in the two formats the NKG2D ectodomain formed a functional dimer and resulted in similar efficient tumor cell cytotoxicity. A control monospecific that binds to NKG2D ligands but not CD3, 13-14c (FIG. 2A), did not result in appreciable cytotoxicity. Thus, these different designs enable potent tumor cell killing by targeting NKG2D ligands and therefore can be developed to treat a broad range of tumor types.

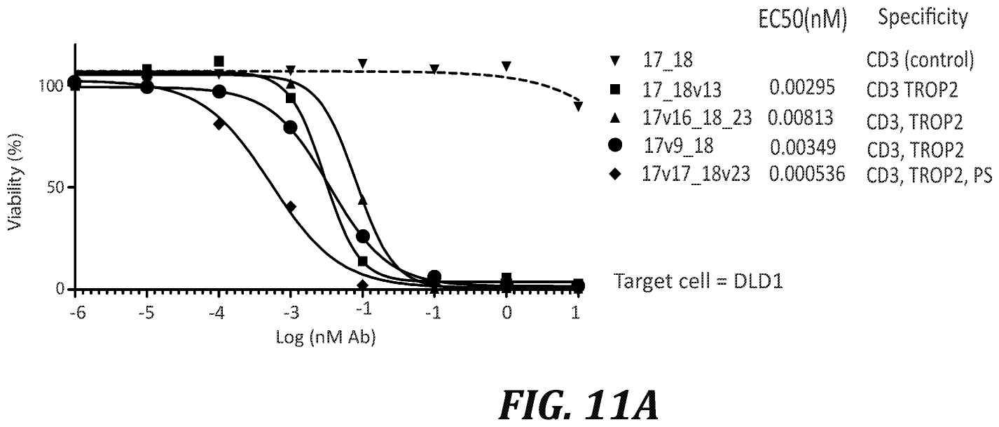

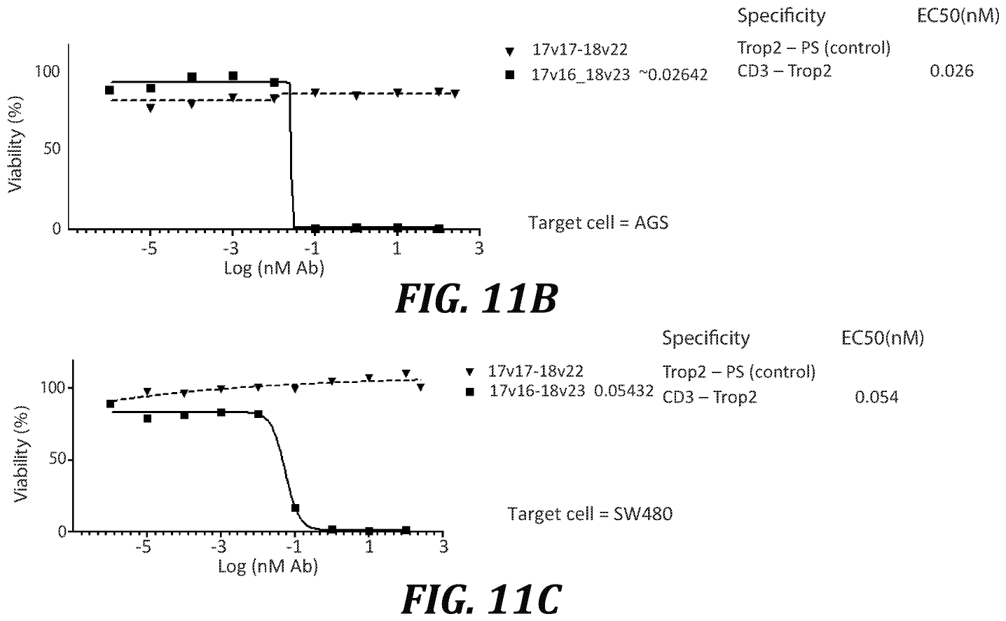

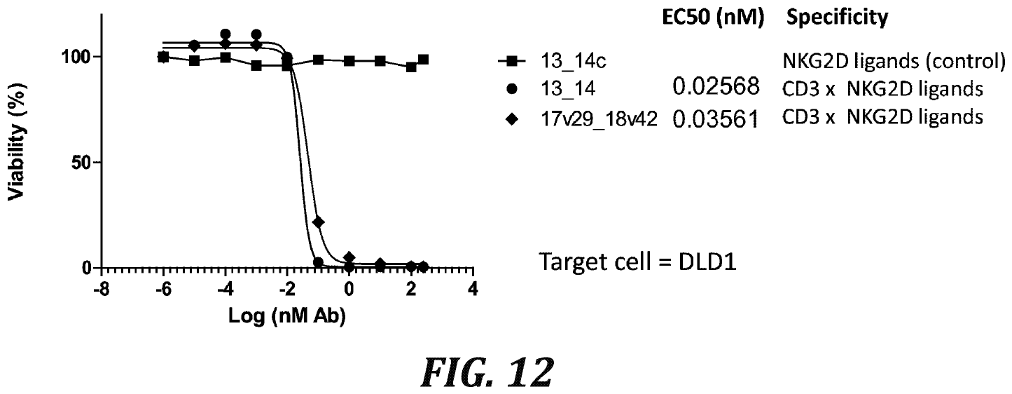

There are eight human NKG2D ligands, MICA, MICB and ULBP1-6, which are expressed broadly on various tumor types and therefore collectively can be considered universal tumor antigens. Thus, these NKG2D bispecific recombinant antigen binding molecules can be developed as therapeutics for many different cancer types including lung, colorectal, stomach, liver and breast. NKG2D ligands are upregulated with stress that also occurs with virus infection and thus they can be targeted to kill or phagocytosis virally infected cells. NK cell activation and the targeting NKG2D ligands is subject to several NK inhibitory receptors (Fuertes M B, Domaica C I, Zwirner N W. Leveraging NKG2D Ligands in Immuno-Oncology. Front Inmmunol. 2021 Jul. 29; 12:713 158). The NKG2D multispecific recombinant molecules circumvent these mechanisms of NK cell inhibition. Furthermore, with the addition of a PS binding domain, such as b2GP1 domain 5 or Tim4, a trispecific recombinant antigen binding molecule can be developed with greatly enhanced potency (as observed for a Trop2 trispecific recombinant antigen binding molecule; FIG. 11A). The increase in avidity with PS binding can be of particular importance to enable effective targeting as NKG2D ligands are characteristically expressed at low density (FIG. 6) and tumor and virally infected cells can further downmodulate their expression to decrease NK cell targeting (Fuertes M B, Domaica C I, Zwirner N W. Leveraging NKG2D Ligands in Immuno-Oncology. Front Immunol. 2021 Jul. 29; 12:713158). Designing a bivalent NKG2D recombinant antigen binding molecule that can bind all NKG2D ligands is difficult as monovalent NKG2D is a dimer. Therefore, the increase in avidity with adding a small domain, such as b2GP1 domain 5, is a crucial to enable generation of an effective multispecific to support targeting tumor or virally infected cell NKG2D ligands. Targeting both NKG2D ligands and PS can result in less off-tumor cytotoxicity as a relatively low dose can be identified that selectively targets tumor cells. Finally, synergy and greater efficacy can result from blocking PS which is an immune checkpoint (Birge R B, Boeltz S, Kumar S, Carlson J, Wanderley J, Calianese D, Barcinski M, Brekken R A, Huang X, Hutchins J T, Freimark B, Empig C, Mercer J, Schroit A J, Schett G, Herrmann M. Phosphatidylserine is a global immunosuppressive signal in efferocytosis, infectious disease, and cancer. Cell Death Differ. 2016 June; 23(6):962-78).

### Enhancing Tumor Cell Cytotoxicity of a Bispecific Antigen Binding Protein Through Utilization of an IgM CH1.

Three bispecific TetrAx recombinant antigen binding molecules were generated that possess an N-terminal modified Fab that binds CD3 and C-termini that possess b2GP1 domains for bivalent binding to PS (FIG. 2A). The three molecules differed in possessing either an IgM, IgA1 or IgG1 CH1. These were produced and purified as described in Example 1. Their ability to mediate DLD1 tumor cell cytotoxicity was compared in a cytotoxicity assay as described in for NKG2DL targeted T cell engaging bispecifics. All demonstrated potent DLD1 cytotoxicity (sub-nM EC50), however the TetrAx recombinant antigen binding molecule with an IgM CH1 (MCH1), which was approximately 10,000-fold more potent than that with an IgG1 CH1 (G1CH1) and 100,000-fold more potent than that with an IgA1 CH1 (A1CH1) (FIG. 8). The same molecules, but lacking b2GP1 domains, do not support appreciable cytotoxic activity (FIG. 8). The order of potency; MCH1>>G1CH1>A1CH1 roughly reflects the level of their binding to DLD1 (FIG. 6A). However, an MCH1 TetrAx that is identical, except for a single amino acid substitution to eliminate the CH1 N-linked glycosylation site (NNS/NNA), binds at similar levels to that of the WT MCH1 TetrAx (FIG. 6A) but demonstrates 100,000-fold less cytotoxic activity (FIG. 8). The binding of the MCH1 WT TetrAx molecule to T cells is equivalent to that of G1CH1 possessing TetrAx whereas the A1CH1 possessing TetrAx demonstrates a lower MFI (FIG. 6D).

The data demonstrate another property by which multispecific recombinant antigen binding molecules comprised of an MCH1 can be advantaged. In addition to production, MCH1 TetrAx can demonstrate substantially greater tumor cytotoxic activity relative to those that possess A1CH1 or G1CH1. The mechanism can in-part involve greater target cell binding.

Enhancing Tumor Cell Cytotoxicity of Bispecific Recombinant Antigen Binding Molecules with the Addition of PS Targeting.

The tumoricidal activity of four T cell engaging bi- or trispecific recombinant antigen binding molecules targeting TROP2 or TROP2 and phosphatidylserine (PS) was determined in an 18-hour assay with T cells and DLD1 as described for NKG2D T cell engagers. Two TetrAx possessed an N-terminal CD3 binding domain and a C-terminal Trop2 binding scFv either on the light (17v9-18) or heavy chain (17-18v13). A third TetrAx possessed an N-terminal TROP2 binding domain and a C-terminal CD3 binding scFv (17v16-18v23). The fourth TetrAx possesses an N-terminal TROP2 binding domain and C-terminal CD3 (scFv) and a PS (b2GP1 domain 5) binding domains (17v17-18v23). The expression of TROP2 on DLD1 and the binding of the bi- and trispecific recombinant antigen binding molecules to DLD1 and T cells, is depicted in FIG. 6. Similar potent levels of DLD1 cytotoxicity (EC50 approximately 0.003 nM) were observed for 17-18v13 and 17v9-18 (FIG. 11). Slightly lower potency (EC50=0.008 nM) was observed for 17v16-18v23. The potency of the Trop2×CD3 bispecific, 17v16-18v23, was increased 10-fold (EC50=0.0005 nM) through the addition of the PS-binding domain (17v17-18v23). The TROP2 T cell engager 17v16-18v23 also demonstrates potent gastric (AGS) and CRC (SW480) tumor cell cytotoxicity (FIGS. 11B and 11C). The control TetrAx, 17-18, that binds only CD3, did not support appreciable cytotoxicity. The binding (MFI) to DLD1 increased with the addition of the PS-binding domain (FIG. 6). Thus, it is demonstrated that specific tumor targeting and killing is enhanced through more avid multispecific binding to tumor cells resulting from the additional binding to a universal tumor antigen, PS. This trispecific recombinant antigen binding molecules, 17v17-18v23 can be developed for several cancer types that express Trop2 and PS including colorectal, stomach, lung, and breast.

Combining specificity for a tumor associated antigen with a universal tumor antigen can be broadly enabling. As most tumor associated antigens are also expressed on normal cells, efficacious dosing of typical T cell engaging bispecific recombinant antigen binding molecules can be limited by off-tumor toxicity. Increasing avidity by targeting a single tumor antigen in a bivalent fashion will also enhance binding to normal cells that express the antigen in common. However, there is no increase in binding to normal cells with targeting two distinct antigens that aren't co-expressed on normal cell types. Since targeting a tumor cell signature such as Trop2 plus PS results in greater potency, a relatively low but efficacious dose can be identified. That is, a lower dose can be identified that favors tumor targeting and cytotoxicity over normal cells. Therefore, this dual antigen targeting can broadly enable greater on-tumor and decreased off-tumor cytotoxicity in the treatment of several different cancer types.

In addition to increasing tumor cytotoxicity through avidity, targeting PS can enable a second mechanism of activity. Sufficient binding to PS can block its immune checkpoint activity. For example, it can compete with Tim3 binding to PS that leads to suppression of T cell cytotoxicity (Wolf Y, Anderson A C, Kuchroo V K. TIM3 comes of age as an inhibitory receptor. Nat Rev Immunol. 2020 March; 20(3):173-185. doi: 10.1038/s41577-019-0224-6. Epub 2019 Nov. 1. PMID: 31676858; PMCID: PMC7327798). Hence synergy and greater efficacy can result from blocking PS. Combining a universal tumor antigen such as PS can enable targeting various tumor associated antigens (TAA) for greater efficacy, tumor specificity and safety.

In constructing a multispecific recombinant antigen binding molecule targeting two tumor antigens, low affinity binding domains are selected for each individual target. The affinity of b2GP1 domain 5 is low (0.2 μM) relative to most clinical stage tumor targeting antibodies. B2GP1 domain 5 (residues A261 to C344) is also small (9 kDa), which can result in greater tumor penetration relative to certain other PS ligands. In addition, certain PS receptors such as Tim3 or 4, Stabilin 1 or 2, RAGE, and CD300a may not be PS specific and have several ligands, and depend on in vivo Ca²⁺ concentrations for binding, which can be negligible in the TME. A PS specific receptor such as B2GP1 is thus ideal to pair with a low affinity binding domain for a second tumor associated antigen to generate tumor specific multispecific recombinant antigen binding molecules.

Another antigen that can be considered a universal tumor antigen is B7H3 as it is broadly and highly expressed on most tumor types and expressed at lower levels on many normal cell types (Zhou W T, Jin W L. B7-H3/CD276: An Emerging Cancer Immunotherapy. Front Immunol. 2021 Jul. 19; 12:701006. doi: 10.3389/fimmu.2021.701006. PMID: 34349762; PMCID: PMC8326801). Like PS, B7H3 also functions as an immune checkpoint. A multispecific with low affinity for B7H3 and PS can therefore more selectively, safely and efficaciously target tumor cells, at lower doses. In addition, a T cell, or tumor cell DR6 engager, targeting both PS and B7H3 can demonstrate high efficacy due to synergistic checkpoint inhibition. These trispecific recombinant antigen binding molecules that target two tumor antigens, where one or both are universal target antigens and immune check points are an improvement, relative to their counterparts that are bispecific but target a single tumor antigen.

The tumor cell cytotoxicity of T cell engagers is markedly enhanced by bivalent PS targeting.

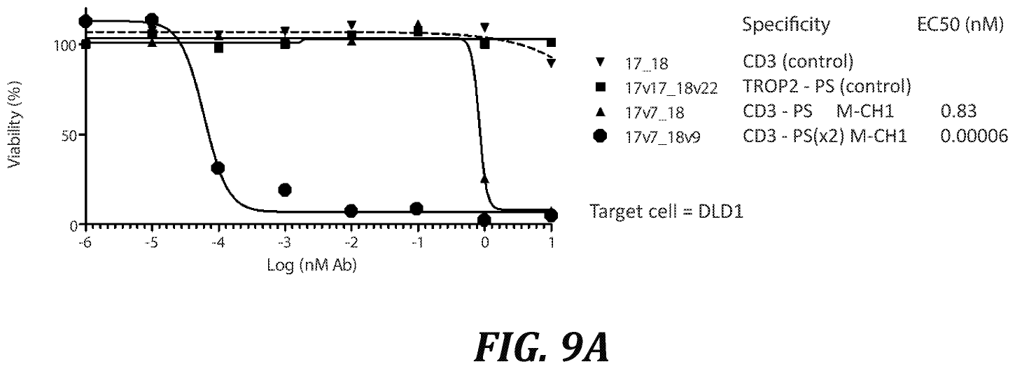

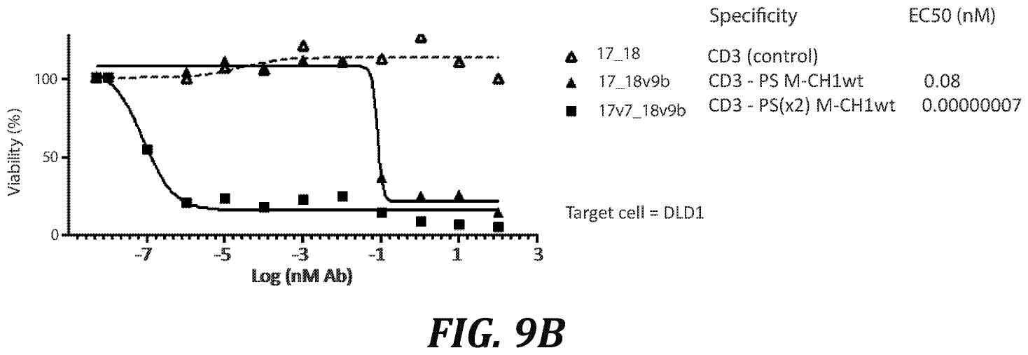

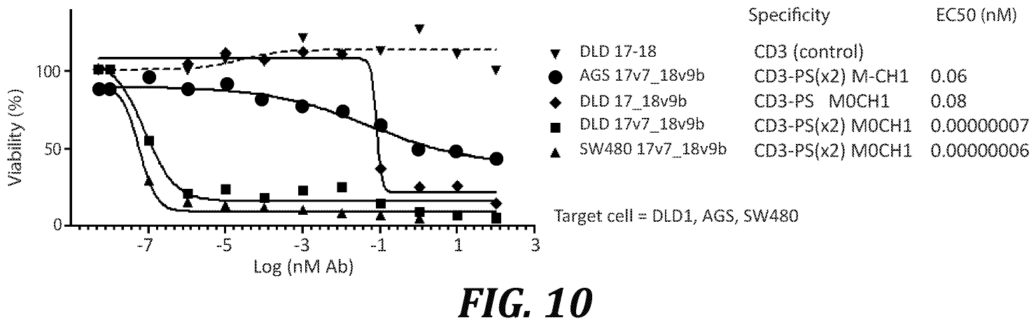

The tumoricidal activity of T cell engaging bispecifics with monovalent or bivalent phosphatidylserine (PS) binding was determined in an 18-hour assay with T cells and DLD1 as described for NKG2DL T cell engagers. TetrAx that have monovalent CD3 binding and either monovalent (18v9b) or bivalent (17v7-18v9b) PS binding were compared. The binding of TetrAx bivalent for PS was substantially greater relative to monovalent binding, as determined by flow fluorimetry (FIG. 6A). Bivalent PS targeting resulted in 13,000-fold greater DLD1 cytotoxicity (EC50=0.00006 nM) relative to the TetrAx with monovalent PS binding (EC₅₀=0.8 nM) (FIG. 9A). In a repeat assay, bivalent PS targeting resulted in >100,000-fold greater DLD1 cytotoxicity (EC50=0.00000007 nM) relative to the TetrAx with monovalent PS binding (EC₅₀=0.08 nM), (FIG. 9B). The control TetrAx, 17-18, which is monospecific and monovalent for CD3, did not support appreciable cytotoxicity.

Targeting CD3 binding to tumor cell PS using b2GP1 domain 5 in a bivalent format results in exceptionally high target cell binding and cytotoxicity (fM EC₅₀). The large increase in cytotoxicity relative to monovalent PS binding, can be due to not only greater avidity, but also blocking the immune checkpoint activity of PS (Birge R B, Boeltz S, Kumuar S. Carlson J, Wanderley J, Calianese D, Barcinski M, Brekken R A, Huang X, Hutchins J T, Freimark B, Empig C, Mercer J, Schroit A J, Schett G, Herrmann M. Phosphatidylserine is a global immunosuppressive signal in efferocytosis, infectious disease, and cancer. Cell Death Differ. 2016 June; 23(6):962-78). The TetrAx format used to generate the bivalent PS binding molecules (FIG. 2A) allowed for efficient production and exceptionally high tumor cytotoxic activity.

### T Cell Signaling Induced by One or Two CD137L Monomers

TetrAx specific for Trop2 were produced with either one, 20v8-18v22(A) or two, 20v8-18v26 (B) C-terminal CD137L domains (SEQ ID NOs: 1-3). For the TetrAx with two CD137L domains, one CD137L domain is attached through a flexible linker to the C-terminus of the 20v8 subunit and the other through a flexible linker to the C-terminus of the 18v26 subunit. Their general format is depicted in FIG. 2A. Each monomeric CD137L domain is composed of the tertiary ectodomain residues 90 to 242; i.e., the TNF SF homology domain (THD). To stimulate signaling through the CD137 receptor it must be crosslinked by CD137L, which in its native configuration is a trimer that is presented on a cell surface in an avid fashion. To demonstrate that the ability the TetrAx to induce CD137 signaling is dependent on binding to a Trop2 expressing tumor cell, the TetrAx and a CD137 reporter cell line (Promega) were co-incubated for 6 hours with or without the Trop2 expressing DLD-1 colon cancer cells. CD137 signal transduction in the reporter cell line activates NF-kB which binds to response elements linked to a promoter for induction of luciferase expression. Signaling activity was determined after 6 hours of co-incubation by adding luciferase substrate and three measurements using a multimode plate reader. Both TetrAx significantly induced signaling in the presence of DLD1 (FIGS. 14A and 14B). Relatively low or negligible levels of signaling was observed in the absence of DLD1 (dashed line). As an additional control, TetrAx that possessed a targeting domain for Trop2, but lacked CD137L domains (17v6-18v25), and in the presence of DLD1, did not stimulate CD137 signaling. Thus, in this system, the binding of these TetrAx to DLD1 Trop2 appears to provide a surface presenting multivalent CD137L, that is essential to bind and crosslink CD137 and stimulate signaling.

The TetrAx with two CD137L domains (FIG. 4B) induced at least 3-fold higher signaling FIG. 14B relative to that with a single CD137L domain (FIG. 14A). This is likely due to the two CD137L domains at the C-terminus of 20v8-18v26 forming a dimer at their native interface, resulting in a complete binding site for a CD137 monomer (Won E Y, Cha K, Byun J S, Kim D U, Shin S, Abn B, Kim Y H, Rice A J, Walz T, Kwon B S, Cho I-S. The structure of the trimer of human 4-1 BB ligand is unique among members of the tumor necrosis factor superfamily. J Biol Chem. 2010 Mar. 19; 285(12).9202-10). A single CD137L monomer possesses a partial contact site for a CD137 monomer. Thus, the single CD137L domain would have lower affinity and be less efficient in binding and crosslinking CD137. The relatively robust signaling stimulated by 20v8-18v26 indicates that it can present a functional CD137L dimer with a complete higher affinity CD137 binding interface.

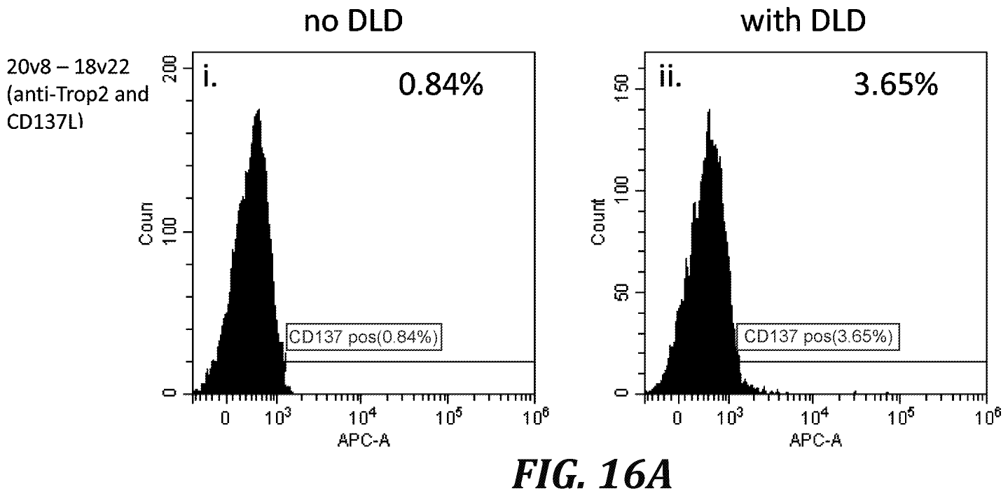

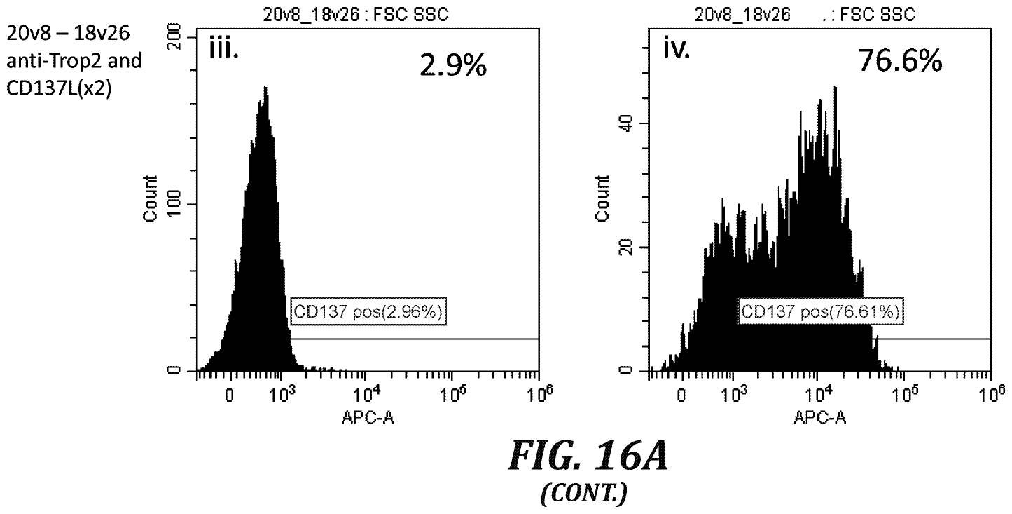

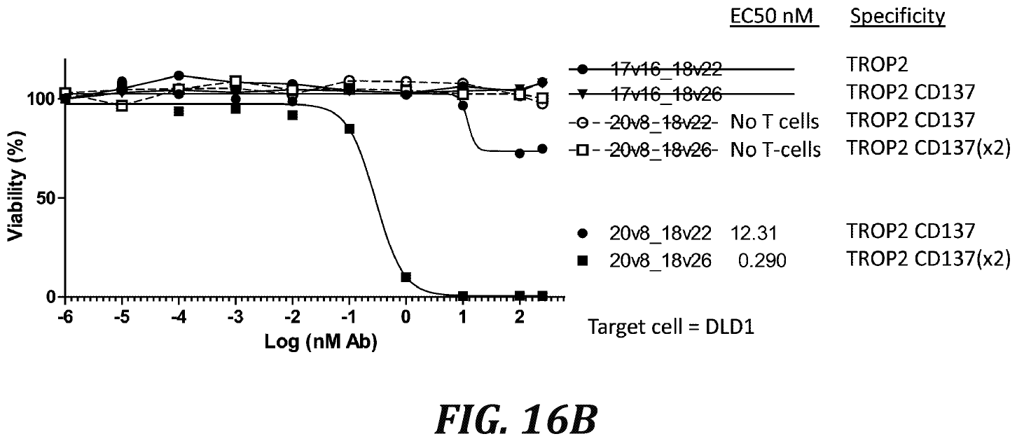

The TetrAx with dual CD137L can also induce T cell CD137 expression and T cell dependent cytotoxic activity, without engaging CD3 (FIGS. 16A and 16B). The TetrAx molecule that targets Trop2 and possesses either one (20v8-18v22) or two (20v8-18v26) CD137L domains were incubated (at 20 nM) with T cells for 18 hours in the presence or absence of DLD1. The TetrAx molecule possessing a single CD137L did not induce substantial T cell CD137 expression regardless of the presence of DLD1 (FIG. 16A; top panels). The TetrAx with the CD137L dimer robustly induced T cell CD137 expression but only in the presence of DLD1 (FIG. 16A bottom panels). In the T cell DLD1 cytotoxicity assay, the TetrAx with a CD137L dimer mediated DLD1 cytotoxicity with an EC₅₀=0.3 nM (FIG. 16B). The single domain TetrAx appeared to support a low level of DLD1 cytotoxicity at 40-fold higher concentrations (FIG. 16B). Thus, although a single CD137L can induce CD137 signaling, its relatively low level of signaling, compared to that stimulated with a CD137L dimer, it does not efficiently induce CD137 expression or T cell dependent tumor cell cytotoxicity. In contrast, a TetrAx molecule with two CD137L domains can effectively stimulate upregulation of CD137 expression and T cell dependent Tumor cell cytotoxicity.

The mechanism of 20v8-18v26 stimulated tumor cytotoxicity can be through CD137 mediated T cell activation leading to TNFa secretion, resulting in induction of DLD1 apoptosis. Alternatively, as NKG2D can be expressed on expanded T cells, DLD cytotoxicity might result from activation of NKG2D mediated granzyme/perforin release. Therefore, 20v8-18v26 can represent a class of TetrAx that can directly induce tumor cell death and stimulate an anti-tumor immune response without the potential safety risks of a CD3 T cell engager. A TetrAx recombinant antigen binding molecule that possesses two CD137L domains and is bivalent for PS can be a potent member of this class with universal tumor targeting.

Thus, a CD137L trimer is not essential for robust signaling and function, and a smaller dimer as designed, can serve as an efficient CD137 agonist. Moreover, its activity is demonstrated to be target antigen (TROP2) dependent and thus capable of localizing activity to a site where cells expressed the target antigen such as a tumor microenvironment (TME). The dual CD137L TetrAx is advantaged in that it enables induction of tumor cell cytotoxicity without the toxicities associated with direct CD3 engagement such as cytokine release syndrome. The dual CD137L TetrAx can also be expected to support other CD137 associated activities. CD137 agonists can induce T and NK proliferation, IFN-g production and activate cytotoxic activity (Hashimoto K. CD137 as an Attractive T Cell Co-Stimulatory Target in the TNFRSF for Immuno-Oncology Drug Development. Cancers (Basel). 2021 May 11; 13(10):2288). CD137 can activate a monocyte and macrophage inflammatory, anti-tumor response (Stoll A, Bruns H, Fuchs M, Völkl S, Nimmerjahn F, Kunz M, Peipp M, Mackensen A, Mougiakakos D. CD137 (4-1BB) stimulation leads to metabolic and functional reprogramming of human monocytes/macrophages enhancing their tumoricidal activity. Leukemia. 2021 December; 35(12):3482-3496). CD137 is an important target for reinvigoration of exhausted tumor-infiltrating lymphocytes or TIL (Kim H D, Park S, Jeong S, Lee Y J, Lee H, Kim C G, Kim K H, Hong S M, Lee J Y, Kim S, Kim I K, Min B S, Chang J H, Ju Y S, Shin E C, Song G W, Hwang S, Park S H. 4-1BB Delineates Distinct Activation Status of Exhausted Tumor-Infiltrating CD8⁺ T Cells in Hepatocellular Carcinoma. Hepatology. 2020 March; 71(3):955-971).

The demonstration that the C-terminal TetrAx CD137L can apparently form functional dimers enables the rational design and generation of small targeted TNFSF ligands (StaT, FIG. 2D-2I) for greater efficacy due to increased tumor penetration. These molecules can be targeted to tumor associated antigens (TAA) or a combination of TAA and universal tumor antigens such as PS. Small, single CD137L domain molecules, stimulating weaker CD137 signaling and functions, can also potentially demonstrate in vivo activity over time. The native TNFSF ligand interface can be modified to stabilize monomers or dimers (see Modifications of CD137L). Although, circulatory half-life of these smaller molecules can be short, several administrations can lead to an anti-tumor immune response. TetrAx or STaT with a single or dual CD137L can be used to fine tune CD137 responses more predictably, relative CD137 antibodies.

Trimeric TFNSF ligands, such as TRAIL, CD40L, LIGHT, and TNFa can also be targeted to universal and more specific tumor antigens to support tumor cytotoxicity and/or an anti-tumor immune response.

A single CD137L domain stimulates robust CD137 signaling when linked to CD3 binding.

TetrAx can be generated that can serve as potential immune adjuvants. One design can for example, target a CD137L dimer to a universal tumor antigen such as PS. A more general, design can include monovalent CD3 binding coupled with weak CD137 co-stimulation. Thus, a TetrAx with monovalent CD3 binding was produced with one C-terminal CD137L domain (17-18v14). The TetrAx was incubated for 6 hours with the CD3 expressing, CD137 reporter cell line. This TetrAx supported signaling (FIG. 15A), but apparently not as robust as the Trop2 targeted dual CD137L TetrAx (FIG. 14B). This signal was dependent on CD3 and CD137 engagement as TetrAx that possessed a CD137L domain but lacked a CD3 binding domain (20v8-18v22), or possessed a CD3 binding domain but lacked CD137L (17-18), did not induce a signaling response. Thus, with monovalent CD3 binding a single CD137L domain appears to be functional for CD137 signaling. Although CD137L induces NF-kB signaling and CD3 signal transduction induces NFAT activation, co-signaling appears to contribute such that a single CD137L can be a sufficient and effective CD137 agonist. Incubation of 17-18v14 with the expanded T cells led to a weak AICD response at high concentrations (FIG. 15B). Controls include a TetrAx that only bind CD3 and either possesses an IgM CH1 (17-18) or IgG1 CH1 (17-19). The control results indicate that an IgG1 CH1 TetrAx can induce AICD under conditions where an IgM CH1 TetrAx does not. Thus, a TetrAx with a single CD137L domain and monovalent CD3 binding can be developed and potentially dosed to induce a safe T cell CD137 response that can be used to enhance an anti-tumor therapy.

Combining a CD3 domain with a Trop2, CD137 specific molecule provides an additional example of a trispecific with multiple functions. For example, linking an anti-CD3scFv to the C-terminus of 18v22 can be used to create the Trop2, CD3, CD137 trispecific; 20v8-18v23. The addition of CD3 specificity enhanced the single CD137L signaling activity of 20v8-18v22 (specific for Trop2, CD137) in a side-by-side comparison (FIG. 15C). The trispecific demonstrated sub-nM cytotoxicity for DLD1 whereas the Trop2, CD137 specific 20v8-18v22 was not active (FIG. 111D). In addition to tumor cell cytotoxicity, the enhanced CD137 T cell signaling can result in cytokine production to support an anti-tumor immune response. Hence the TetrAx platform can be utilized to create multispecific molecules with greater functionality.

### Mechanism of Tumor Target Antigen-Dependent CD137 Activation

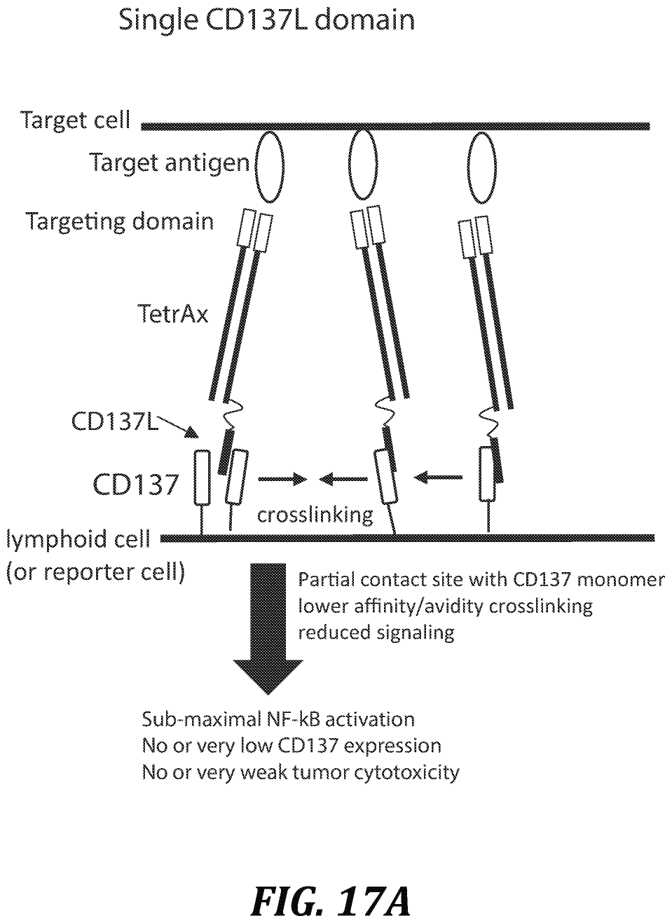

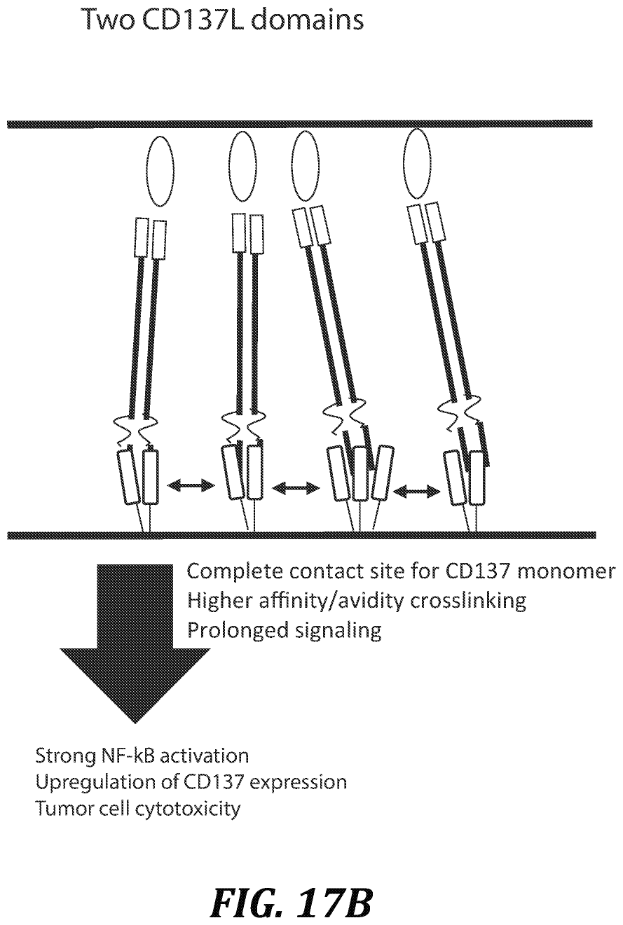

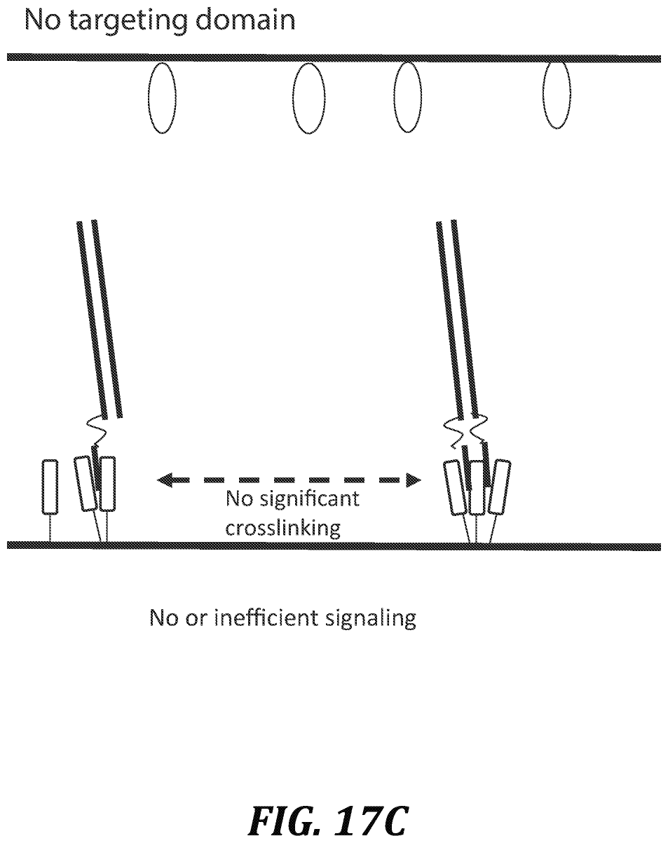

The binding between immune cells can result in the interaction between native trimeric CD137 ligand and its trimeric receptor leading to efficient crosslinking and signaling. The complete contact site for a single CD137 monomer involves two CD137L domains i.e., a CD137L dimer (Bitra A, Doukov T, Croft M, Zajonc D M. Crystal structures of the human 4-1BB receptor bound to its ligand 4-1BBL reveal covalent receptor dimerization as a potential signaling amplifier. J Biol Chem. 2018 Jun. 29; 293(26).9958-9969). It is demonstrated here that TetrAx possessing one or two CD137L domains are capable of inducing CD137 signaling. Signaling is dependent on the TetrAx being able to bind to a cell expressing a target antigen, such as the tumor antigen Trop2 or PS. Once bound, the tumor cell can present TetrAx CD137L in a multivalent fashion to CD137 expressing effector cells. This presentation appears to be sufficient for clustering or crosslinking of CD137 necessary to stimulate signaling (FIG. 17). A TetrAx recombinant antigen binding molecule possessing a single CD137L does not possess a complete CD137 contact interface and therefore is expected to have reduced affinity for CD137, relative to a dimer or trimer. Thus, it can result in relatively transient binding and lower levels of crosslinking compared to two CD137L domains.

Indeed, the signaling induced by TetrAx recombinant antigen binding molecules with a single domain is not as robust as that with two CD137L domains, unless it is also linked to a CD3 binding domain which can result in co-signaling (FIGS. 14 and 17). Furthermore, a tumor antigen targeted bispecific with a single CD137L domain does not induce substantial CD137 expression or T cell cytotoxicity which is observed for TetrAx with two CD137L domains (FIG. 17). These data indicate that the bispecific recombinant antigen binding molecule two domain design allows for a functional dimer to form with a complete CD137 binding interface capable of effectively binding and crosslinking CD137 when presented on the target cell. Thus the selective targeting of one or two CD137L domains can fine tune the localization and magnitude of CD137's contribution to an immune response that can be difficult to recapitulate with CD137 antibodies. This can also apply to the targeting of certain other TNFSF ligands. Substitutions at their monomer-monomer contact interface can allow for the generation of stable homogenous multispecific recombinant antigen binding molecules with one or two monomers with different signaling and function activating properties.

Enhanced efficacy and safety of these multispecific recombinant antigen binding molecules can result from their ability to localize activity to the target tissue. Localized targeting of TNFSF ligands or T cell engagers (CD3 binders) to tumor cells is dependent on their affinity for target antigens and the upregulation of TAA in tumors. For example, normal cells that lack or have low levels of target antigen expression can result in relatively low CD137 signaling and therefore activity would be more restricted to tumor tissue with upregulated target antigen.

High avidity tumor targeting through binding to two tumor target antigens such as PS and TROP2 can also direct most activity to tumor tissue. This can be optimized by using low affinity binding domains for each target such that only the combined avidity efficiently targets tumor cells and supports potent activity. The low affinity of a single b2GP1 domain 5, 0.2 μM, is ideal to pair with a second low affinity TAA targeting domain. Thus b2GP1 can be advantageous relative to higher affinity PS receptors.

Targeting tumor antigens that are also immune checkpoints, such as PS and B7H3, can greatly enhance the potency of a multispecific. The increase in potency resulting from dual targeting and checkpoint inhibition can result in identification of lower efficacious doses that don't significantly bind normal tissues that express only one of the two antigens.

Examples of a Modified CD137L, TRAIL, NKG2D and b2GP1.

Certain modifications to native ligand or receptor domains can facilitate manufacturing and potentially efficacy by increasing stability, increasing affinity and in the case of TNFSF ligands, decreasing spontaneous trimerization of a monomer or dimer that could result in a heterogenous product. For example, CD137L substitutions, E156C and A225C, were modeled based on the NIH Protein Database CD137L structure 6d3n. The inserted cysteines are proximal to, but not within a CD137L-CD137 contact site as described by Bitra (Bitra A, Doukov T, Croft M, Zajonc D M. Crystal structures of the human 4-11B receptor bound to its ligand 4-1BBL reveal covalent receptor dimerization as a potential signaling amplifier. J Biol Chem. 2018 Jun. 29; 293(26):9958-9969).

The residues contributing to the monomer-monomer interface of CD137L and for TRAIL have been identified. Substituting these or other interface residues can lower trimerization at high concentrations without interfering with signaling activity. For example, one chain of a multispecific recombinant antigen binding molecule can possess a wild type CD137L monomer sequence whereas the second can have interface substitutions. Hence, a CD137L dimer can efficiently form but there is decreased ability of the mutant subunit to form a contact with a third monomer. Substitutions of residues in the subunit contact interface of other TNFSF ligands can also enable generation of multispecific recombinant antigen binding molecules with receptor binding agonistic monomers or dimers.

TetrAx can potently kill tumor cells in a 3D tumor model.

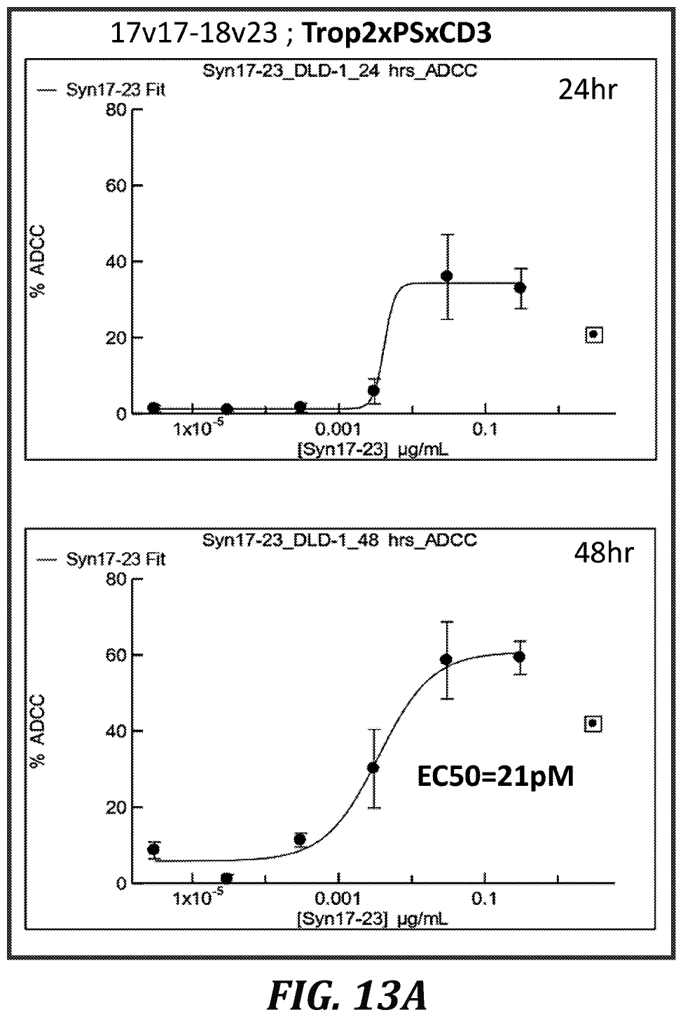

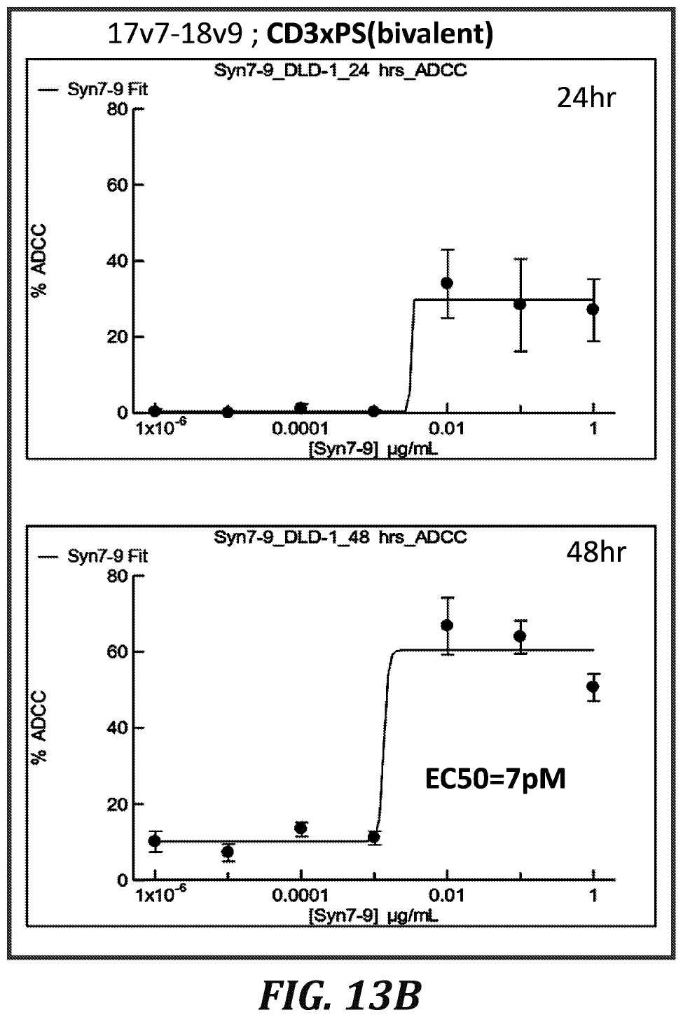

An in vitro 3D tumor model can be more predictive of cytotoxicity activity in vivo relative to the standard 2D assay (Guzzeloni V, Veschini L, Pedica F, Ferrero E, Ferrarini M. 3D Models as a Tool to Assess the Anti-Tumor Efficacy of Therapeutic Antibodies: Advantages and Limitations. Antibodies (Basel). 2022 Jul. 8; 11(3):46.). The cytotoxic activity of several TetrAx recombinant antigen binding molecule candidates was therefore determined in a 3D model using DLD1 tumor cells. These TetrAx recombinant antigen binding molecules are specific for TROP2, PS and CD3 (17v17-18v23) and CD3 and PS(bivalent)—17v7-17v9. DLD-1 cell line cells were grown as spheroids in Elplasia microplates. DLD-1 tumor target cells were labeled with green fluorescing Calcein-Am dye reagent (Biotium cat. #30002) according to the manufacturer's instructions. On the day of the assay the TetrAx recombinant antigen binding molecules were serially diluted by log-dilution and added to the assay plates containing the DLD-1 cells. Effector T cells were then added to the assay plates to achieve a 2:1 effector:target (E:T) ratio. Plates were incubated for 24 hours at 37 C in a 5% C02 incubator. Red fluorescing Dead dye reagent (Biotium cat. #30002) was then added to assay plates according to the manufacturer's instructions and staining was recorded at 24 and 48 hours using a Lionheart Automated Microscope. DLD-1 (Calcein-AM staining) cells co-localized with red-dye (Dead Dye from Biotium) were identified, and the percentage of cytotoxicity was calculated as the population of specific dead-DLD-1 cells (percent of dead DLD-1/Effector cells). All tests were performed in triplicate, and the results were expressed as % cytotoxicity mean±standard deviation (FIG. 13).

The data indicate that 17v7-18v23 (EC₅₀=21 pM) and 17v7-18v9 (EC₅₀=7 pM) mediate potent DLD1 cytotoxicity in a 3D tumor model. The level of cytotoxicity increases from 24 to 48 hours (35-60%). Both TROP2 and PS target antigens appear to be exposed on DLD1 when grown as a 3D tumor. However, tumor cytotoxicity is reduced in the 3D model relative to the 2D model (FIGS. 9 and 11) indicating that the exposure of the target, PS, may be reduced when DLD1 tumor cells are grown in a 3D format. Plasma membrane levels of tumor cell PS can be increased in vivo by treatment with agents that increase tumor stress such as Gemcitabine or radiation (Beck A W, Luster T A, Miller A F, Holloway S E, Conner C R, Barnett C C, Thorpe P E, Fleming J R, Brekken R A. Combination of a monoclonal anti-phosphatidylserine antibody with gemcitabine strongly inhibits the growth and metastasis of orthotopic pancreatic tumors in mice. Int J Cancer. 2006 May 15; 118(10):2639-43; N'Guessan K F, Davis H W, Chu Z, Vallabhapurapu S D, Lewis C S, Franco R S, Olowokure O, Ahmad S A, Yeh J J, Bogdanov V Y, Qi X. Enhanced Efficacy of Combination of Gemcitabine and Phosphatidylserine-Targeted Nanovesicles against Pancreatic Cancer. Mol Ther. 2020 Aug. 5; 28(8):1876-1886; Raoufi Rad N, McRobb L S, Zhao Z, Lee V S, Patel N J, Qureshi A S, Grace M, McHattan J J, Amal Raj J V, Duong H, Kashba S R, Stoodley M A. Phosphatidylserine Translocation after Radiosurgery in an Animal Model of Arteriovenous Malformation. Radiant Res. 2017 June; 187(6):701-707).

While illustrative embodiments have been illustrated and described, it will be appreciated that various changes can be made therein without departing from the spirit and scope of the invention.

TABLE 7
Amino acid sequences

| Construct/ | |
|---|---|
| sequence | SEQ ID NO: |
| CL: RS7scFv-linker-hSP34Vk, CL, linker-hinge, CH2—CH3 (hole) | 1 |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGGGGSGGGGSGGGGSDI
QLTQSPSSLSASVGDRVSITCKASQDVSIAVAWYQQKPGKAPKLLIYSASYRYTGVPDRFSGSGSGTDFTLTI
SSLQPEDFAVYYCQQHYITPLTFGAGTKVEIKASGGGGGQAVVTQEPSLTVSPGGTVTLTCGSSTGAVTTSN
YANWVQQKPGQAFRGLIGGTNKRAPGTPARFSGSLLGGKAALTLSGAQPEDEAEYYCALWYSNLWVFGG
GTKLTVLRTVAAPSVFIFPPSDEQLKSGTASVVCLLNNFYPREAKVQWKVDNALQSGNSQESVTEQDSKDS
TYSLSSTLTLSKADYEKQKVYACEVTHQGLSSPVTKSFLRGECGGPAGGGGTHTCPPCPAPEAAGGPSVFL
FPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVCTLPPSRDELTKNQVSLSCAVKGFYPSDIAVEWES

| | |
|---|---|
| NGQPENNYKTTPPVLDSDGSFFLVSKLTVDKSRWQQGNVFSCSVMHEALHNHYTQKSLSLSPGK | |
| CH1: hSP34Vh, MCH1wt, linker-hinge, CH2—CH3 (knob)-linker-PD1 | 2 |

EVQLVESGGGLVQPGGSLKLSCAASGFTFNTYAMNWVRQAPGKGLEWVGRIRSKYNNYATYYADSVKG
RFTISRDDSKNTLYLQMNSLKTEDTAVYYCVRHGNFGNSYVSWFAYWGQGTLVTVSSGSASAPTLFPLVS
CENSPSDTSSVAVGCLAQDFLPDSITFSWKYKNNSDISSTRGFPSVLRGGKYAATSQVLLPSKDVMQGTDE
HVVCKVQHPNGNKEKNVPLPGGPAGGGGTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGGGGGGGSPDRPWNPPTFSPALLVVTEGDN
ATFTCSFSNTSESFVLNWYRMSPSNQTDKLAAFPEDRSQPGQDSRFRVTQLPNGRDFHMSVVRARRNDSGT
YLCGAISLAPKAQIKESLRAELRVTEP

| | |
|---|---|
| CL: PD1 linker-CL, linker-hinge, CH2—CH3, linker, RS7scFv | 3 |

SPDRPWNPPTFSPALLVVTEGDNATFTCSFSNTSESFVLNWYRMSPSNQTDKLAAFPEDRSQPGQDSRFRVT
QLPNGRDFHMSVVRARRNDSGTYLCGAISLAPKAQIKESLRAELRVTEPASGGGGGRTVAAPSVFIFPPSDE
QLKSGTASVVCLLNNFYPREAKVQWKVDNALQSGNSQESVTEQDSKDSTYSLSSTLTLSKADYEKQKVYA
CEVTHQGLSSPVTKSFLRGECGGPAGGGGTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEK
TISKAKGQPREPQVCTLPPSRDELTKNQVSLSCAVKGFYPSDIAVEWESNGQPENNYKTTPPVLDSDGSFFL
VSKLTVDKSRWQQGNVFSCSVMHEALHNHYTQKSLSLSPGKGGGGGSQVQLGQSGSELKKPGASVKVSC
KASGYTFTSYAMNWVRQAPGQGLKWMGWINTYTGETPYTDDFKGRFAFSLDTSVSTAYLQISSLKADDT
AVYFCARGGFGSSYWYFDVWGQGTLVTVSSGGGGSGGGGSGGGGSDIQLTQSPSSLSASVGDRVSITCKA
SQDVSIAVAWYQQKPGKAPKLLIYSASYRYTGVPDRFSGSGSGTDFTLTISSLQPEDFAVYYCQQHYITPLT
FGAGTKVEIK

| | |
|---|---|
| CH1: TREM2, linker, MCH1wt, linker-hinge, CH2—CH3, SP34scFv | 4 |

HNTTVFQGVAGQSLQVSCPYDSMKHWGRRKAWCRQLGEKGPCQRVVSTHNLWLLSFLRRWNGSTAITD
DTLGGTLTITLRNLQPHDAGLYQCQSLHGSEADTLRKVLVEVLAGGGGGPAGGSASAPTLFPL VSCENSPS
DTSSVAVGCLAQDFLPDSITFSWKYKNNSDISSTRGFPSVLRGGKYAATSQVLLPSKDVMQGTDEHVVCKV
QHPNGNKEKNVPLPGGPAGGGGTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAK
GQPREPQVYTLPPCRDELTKNQVSLWCLVKGFYPSDIAVEWESNGQPENNYKTTPPVLDSDGSFFLYSKLT
VDKSRWQQGNVFSCSVMHEALHNHYTQKSLSLSPGKGGGGGGSEVQLVESGGGLVQPGGSLKLSCAASG
FTFNTYAMNWVRQAPGKGLEWVGRIRSKYNNYATYYADSVKGRFTISRDDSKNTLYLQMNSLKTEDTAV
YYCVRHGNFGNSYVSWFAYWGQGTLVTVSSGGGGSGGGGSGGGGSQAVVTQEPSLTVSPGGTVTLTCGS
STGAVTTSNYANWVQQKPGQAFRGLIGGTNKRAPGTPARFSGSLLGGKAALTLSGAQPEDEAEYYCALW
YSNLWVFGGGTKLTVL

| | |
|---|---|
| CL: hRS7scFv, linker, DR5Vk, CL, linker-hinge, CH2—CH3 | 5 |

QVQLGQSGSELKKPGASVKVSCKASGYTFTSYAMNWVRQAPGQGLKWMGWINTYTGETPYTDDFKGRF
AFSLDTSVSTAYLQISSLKADDTAVYFCARGGFGSSYWYFDVWGQGTL VTVSSGGGGSGGGGSGGGGSDI
QLTQSPSSLSASVGDRVSITCKASQDVSIAVAWYQQKPGKAPKLLIYSASYRYTGVPDRFSGSGSGTDFTLTI
SSLQPEDFAVYYCQQHYITPLTFGAGTKVEIKASGGGGGSSSELTQDPAVSVALGQTVRITCQGDSLRSYYA
SWYQQKPGQAPVLVIYGKNNRPSGIPDRFSGSSSGNTASLTITGAQAEDEADYYCNSRDSSGNHVVFGGGT
KLTVLRTVAAPSVFIFPPSDEQLKSGTASVVCLLNNFYPREAKVQWKVDNALQSGNSQESVTEQDSKDSTY
SLSSTLTLSKADYEKQKVYACEVTHQGLSSPVTKSFLRGECGGPAGGGGGGTHTCPPCPAPEAAGGPSVFL
FPPKPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVCTLPPSRDELTKNQVSLSCAVKGFYPSDIAVEWES
NGQPENNYKTTPPVLDSDGSFFLVSKLTVDKSRWQQGNVFSCSVMHEALHNHYTQKSLSLSPGK

| | |
|---|---|
| CH1: DR5Vh, MCH1wt, linker-hinge, CH2—CH3, linker, CD137L | 6 |
| EVQLVQSGGGVERPGGSLRLSCAASGFTFDDYGMSWVRQAPGKGLEWVSGINWNGGSTGYADSVKGRV | |

TISRDNAKNSLYLQMNSLRAEDTAVYYCAKILGAGRGWYFDLWGKGTTVTVSSGSASAPTLFPLVSCENS
PSDTSSVAVGCLAQDFLPDSITFSWKYKNNSDISSTRGFPSVLRGGKYAATSQVLLPSKDVMQGTDEHVVC
KVQHPNGNKEKNVPLPGGPAGGGGTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQPENNYKTTPPVLDSDGSFFLYSK
LTVDKSRWQQGNVFSCSVMHEALHNHYTQKSLSLSPGKGGGGGGMFAQLVAQNVLLIDGPLSWYSDPGL
AGVSLTGGLSYKEDTKELVVAKAGVYYVFFQLELRRVVAGEGSGSVSLALHLQPLRSAAGAAALALTVDL
PPASSEARNSAFGFQGRLLHLSAGQRLGVHLHTEARARHAWQLTQGATVLGLFRVTP

| | |
|---|---|
| CL: NKG2D, linker, CL, linker-hinge, CH2—CH3 (hole), linker, PD1 | 7 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGGGGGRTVAAPS | |
| VFIFPPSDEQLKSGTASVVCLLNNFYPREAKVQWKVDNALQSGNSQESVTEQDSKDSTYSLSSTLTLSKAD | |
| YEKQKVYACEVTHQGLSSPVTKSFLRGECGGPAGGGGGGTHTCPPCPAPEAAGGPSVFLFPPKPKDTLMIS | |
| RTPEVTCVVVDVSHEDPEVKFNWYVDGVEVHNAKTKPREEQYNSTYRVVSVLTVLHQDWLNGKEYKCK | |

VSNKALPAPIEKTISKAKGQPREPQVCTLPPSRDELTKNQVSLSCAVKGFYPSDIAVEWESNGQPENNYKTT

| | |
|---|---|
| PPVLDSDGSFFLVSKLTVDKSRWQQGNVFSCSVMHEALHNHYTQKSLSLSPGKGGGGGGSPDRPWNPPTF | |
| SPALLVVTEGDNATFTCSFSNTSESFVLNWYRMSPSNQTDKLAAFPEDRSQPGQDSRFRVTQLPNGRDFHM | |
| SVVRARRNDSGTYLCGAISLAPKAQIKESLRAELRVTEP | |
| CH1: NKG2D, linker, MCH1wt, linker-hinge, CH2—CH3, linker CD137L | 8 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGGGGGGSASAPT | |
| LFPLVSCENSPSDTSSVAVGCLAQDFLPDSITFSWKYKNNSDISSTRGFPSVLRGGKYAATSQVLLPSKDVM | |
| QGTDEHVVCKVQHPNGNKEKNVPLPGGPAGGGGTHTCPPCPAPEAAGGPSVFLFPPKPKDTLMISRTPEVT | |
| CVVVDVSHEDPEVKFNWYVDGVEVHNAKTKPREEQYNSTYRVVSVLTVLHQDWLNGKEYKCKVSNKAL | |

PAPIEKTISKAKGQPREPQVYTLPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGKGGGGGGMFAQLVAQNVLLIDGP
LSWYSDPGLAGVSLTGGLSYKEDTKELVVAKAGVYYVFFQLELRRVVAGEGSGSVSLALHLQPLRSAAGA

| | |
|---|---|
| AALALTVDLPPASSEARNSAFGFQGRLLHLSAGQRLGVHLHTEARARHAWQLTQGATVLGLFRVTP | |
| CL: hSP34Vk, CL, linker-hinge, CH2—CH3 (hole), PD1 | 9 |
| QAVVTQEPSLTVSPGGTVTLTCGSSTGAVTTSNYANWVQQKPGQAFRGLIGGTNKRAPGTPARFSGSLLGG | |
| KAALTLSGAQPEDEAEYYCALWYSNLWVFGGGTKLTVLRTVAAPSVFIFPPSDEQLKSGTASVVCLLNNF | |
| YPREAKVQWKVDNALQSGNSQESVTEQDSKDSTYSLSSTLTLSKADYEKQKVYACEVTHQGLSSPVTKSF | |
| LRGECGGPAGGGGTHTCPPCPAPEAAGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISKAKGQPREPQVCT | |
| LPPSRDELTKNQVSLSCAVKGFYPSDIAVEWESNGQPENNYKTTPPVLDSDGSFFLVSKLTVDKSRWQQGN | |
| VFSCSVMHEALHNHYTQKSLSLSPGKGGGGGGSPDRPWNPPTFSPALLVVTEGDNATFTCSFSNTSESFVL | |
| NWYRMSPSNQTDKLAAFPEDRSQPGQDSRFRVTQLPNGRDFHMSVVRARRNDSGTYLCGAISLAPKAQIK | |
| ESLRAELRVTEP | |
| CH1: SP34Vh, MCH1wt, linker-hinge, CH2—CH3 (knob), linker, sc VγVδ | 10 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGSASAPTLFPLVS

CENSPSDTSSVAVGCLAQDFLPDSITFSWKYKNNSDISSTRGFPSVLRGGKYAATSQVLLPSKDVMQGTDE
HVVCKVQHPNGNKEKNVPLPGGPAGGGGTHTCPPCPAPEAAGGPSVFLFPPKPKDTLMISRTPEVTCVVVD

VSHEDPEVKFNWYVDGVEVHNAKTKPREEQYNSTYRVVSVLTVLHQDWLNGKEYKCKVSNKALPAPIEK
TISKAKGQPREPQVYTLPPCRDELTKNQVSLWCLVKGFYPSDIAVEWESNGQPENNYKTTPPVLDSDGSFF

LYSKLTVDKSRWQQGNVFSCSVMHEALHNHYTQKSLSLSPGKGGGGSHLEQPQISSTKTLSKTARLECVVS

GITISATSVYWYRERPGEVIQFLVSISYDGTVRKESGIPSGKFEVDRIPETSTSTLTIHNVEKQDIATYYCALW
DRYYKKLFGSGTTLVVTDGGGGSGGGGSGGGGSQKVTQAQSSVSMPVRKAVTLNCLYETSWWSYYIFW

| | |
|---|---|
| YKQLPSKEMIFLIRQGSDEQNAKSGRYSVNFKKAAKSVALTISALQLEDSAKYFCALGVRAFLRDWGIRVLI | |
| FGKGTRVTVEP | |
| CL: RS7Vk, CL, linker-hinge, CH2—CH3 (hole), PD-1 | 11 |
| DIQLTQSPSSLSASVGDRVSITCKASQDVSIAVAWYQQKPGKAPKLLIYSASYRYTGVPDRFSGSGSGTDFT | |
| LTISSLQPEDFAVYYCQQHYITPLTFGAGTKVEIKRTVAAPSVFIFPPSDEQLKSGTASVVCLLNNFYPREAK | |
| VQWKVDNALQSGNSQESVTEQDSKDSTYSLSSTLTLSKADYEKQKVYACEVTHQGLSSPVTKSFLRGECG | |
| GPAGGGGTHTCPPCPAPELLGGPSVFLFPPKPKDTLMISRTPEVTCVVVDVSHEDPEVKFNWYVDGVEVHN | |
| AKTKPREEQYNSTYRVVSVLTVLHQDWLNGKEYKCKVSNKALPAPIEKTISKAKGQPREPQVCTLPPSRDE | |
| LTKNQVSLSCAVKGFYPSDIAVEWESNGQPENNYKTTPPVLDSDGSFFLVSKLTVDKSRWQQGNVFSCSV | |
| MHEALHNHYTQKSLSLSPGKGGGGGGSPDRPWNPPTFSPALLVVTEGDNATFTCSFSNTSESFVLNWYRM | |
| SPSNQTDKLAAFPEDRSQPGQDSRFRVTQLPNGRDFHMSVVRARRNDSGTYLCGAISLAPKAQIKESLRAE | |
| LRVTEP | |
| CH1: RS7Vh, MCH1wt, linker-hinge, CH2—CH3, linker, b2GP1 | 12 |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGSASAPTLFPLVSCENSPS

| | |
|---|---|
| DTSSVAVGCLAQDFLPDSITFSWKYKNNSDISSTRGFPSVLRGGKYAATSQVLLPSKDVMQGTDEHVVCKV | |
| QHPNGNKEKNVPLPGGPAGGGGTHTCPPCPAPELLGGPSVFLFPPKPKDTLMISRTPEVTCVVVDVSHEDPE | |
| VKFNWYVDGVEVHNAKTKPREEQYNSTYRVVSVLTVLHQDWLNGKEYKCKVSNKALPAPIEKTISKAKG | |
| QPREPQVYTLPPCRDELTKNQVSLWCLVKGFYPSDIAVEWESNGQPENNYKTTPPVLDSDGSFFLYSKLTV | |
| DKSRWQQGNVFSCSVMHEALHNHYTQKSLSLSPGKGGGGGGASCKVPVKKATVVYQGERVKIQEKFKN | |
| GMLHGDKVSFFCKNKEKKCSYTEDAQCIDGTIEVPKCFKEHSSLAFWKTDASDVKPC | |
| CL: hSP34Vk, CL | 13 |
| QAVVTQEPSLTVSPGGTVTLTCGSSTGAVTTSNYANWVQQKPGQAFRGLIGGTNKRAPGTPARFSGSLLGG | |
| KAALTLSGAQPEDEAEYYCALWYSNLWVFGGGTKLTVLRTVAAPSVFIFPPSDEQLKSGTASVVCLLNNF | |
| YPREAKVQWKVDNALQSGNSQESVTEQDSKDSTYSLSSTLTLSKADYEKHKVYACEVTHQGLSSPVTKSF | |
| NRGEC | |
| CH1: hSP34Vh, MCH1, linker-hinge, CH2—CH3 (hole) | 14 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGSASAPTLFPLVS

| | |
|---|---|
| CENSNPSSTVA VGCLAQDFLPDSITFSWKYKNNSDISSTRGFPSVLRGGKYAATSQVLLPSKDVAQGTNEH | |
| VVCKVQHPNGNKEKDVPLPAGGGGGGTHTCPPCPAPEAAGGPSVFLFPPKPKDTLMISRTPEVTCVVVDVS | |
| HEDPEVKFNWYVDGVEVHNAKTKPREEQYNSTYRVVSVLTVLHQDWLNGKEYKCKVSNKALPAPIEKTI | |
| SKAKGQPREPQVCTLPPSRDELTKNQVSLSCAVKGFYPSDIAVEWESNGQPENNYKTTPPVLDSDGSFFLVS | |
| KLTVDKSRWQQGNVFSCSVMHEALHNHYTQKSLSLSPGK | |
| Linker: hRS7scFv, linker-hinge, CH2—CH3 (knob), PD1 | 15 |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGGGGSGGGGSGGGGSDI
QLTQSPSSLSASVGDRVSITCKASQDVSIAVAWYQQKPGKAPKLLIYSASYRYTGVPDRFSGSGSGTDFTLTI

SSLQPEDFAVYYCQQHYITPLTFGAGTKVEIKPAGGGGGGTHTCPPCPAPEAAGGPSVFLFPPKPKDTLMIS
RTPEVTCVVVDVSHEDPEVKFNWYVDGVEVHNAKTKPREEQYNSTYRVVSVLTVLHQDWLNGKEYKCK

VSNKALPAPIEKTISKAKGQPREPQVYTLPPCRDELTKNQVSLWCLVKGFYPSDIAVEWESNGQPENNYKT

| | |
|---|---|
| TPPVLDSDGSFFLYSKLTVDKSRWQQGNVFSCSVMHEALHNHYTQKSLSLSPGKGGGGGGSPDRPWNPPT | |
| FSPALLVVTEGDNATFTCSFSNTSESFVLNWYRMSPSNQTDKLAAFPEDRSQPGQDSRFRVTQLPNGRDFH | |
| MSVVRARRNDSGTYLCGAISLAPKAQIKESLRAELRVTEP | |
| CL: Vg, CL | 16 |
| HLEQPQISSTKTLSKTARLECVVSGITISATSVYWYRERPGEVIQFLVSISYDGTVRKESGIPSGKFEVDRIPET | |
| STSTLTIHNVEKQDIATYYCALWDRYYKKLFGSGTTLVVTDGGGGGRTVAAPSVFIFPPSDEQLKSGTASV | |
| VCLLNNFYPREAKVQWKVDNALQSGNSQESVTEQDSKDSTYSLSSTLTLSKADYEKHKVYACEVTHQGLS | |
| SPVTKSFNRGEC | |
| CH1: Vd, MCH1, linker-hinge, CH2—CH3 (hole) | 17 |
| QKVTQAQSSVSMPVRKAVTLNCLYETSWWSYYIFWYKQLPSKEMIFLIRQGSDEQNAKSGRYSVNFKKAA | |

KSVALTISALQLEDSAKYFCALGVRAFLRDWGIRVLIFGKGTRVTVEPGGGGGGSASAPTLFPLVSCENSPS

| | |
|---|---|
| DTSSVAVGCLAQDFLPDSITFSWKYKNNSDISSTRGFPSVLRGGKYAATSQVLLPSKDVMQGTDEHVVCKV | |
| QHPNGNKEKNVPLPAGGGGGGTHTCPPCPAPEAAGGPSVFLFPPKPKDTLMISRTPEVTCVVVDVSHEDPE | |
| VKFNWYVDGVEVHNAKTKPREEQYNSTYRVVSVLTVLHQDWLNGKEYKCKVSNKALPAPIEKTISKAKG | |
| QPREPQVCTLPPSRDELTKNQVSLSCAVKGFYPSDIAVEWESNGQPENNYKTTPPVLDSDGSFFLVSKLTVD | |
| KSRWQQGNVFSCSVMHEALHNHYTQKSLSLSPGK | |
| Linker: hSP34scFv, linker-hinge, CH2—CH3 (knob)-PD-1 | 18 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGGGGSGGGGSGG

GGSQAVVTQEPSLTVSPGGTVTLTCGSSTGAVTTSNYANWVQQKPGQAFRGLIGGTNKRAPGTPARFSGSL
LGGKAALTLSGAQPEDEAEYYCALWYSNLWVFGGGTKLTVLPAGGGGGGTHTCPPCPAPEAAGGPSVFLF
PPKPKDTLMISRTPEVTCVVVDVSHEDPEVKFNWYVDGVEVHNAKTKPREEQYNSTYRVVSVLTVLHQD

WLNGKEYKCKVSNKALPAPIEKTISKAKGQPREPQVYTLPPCRDELTKNQVSLWCLVKGFYPSDIAVEWES

| | |
|---|---|
| NGQPENNYKTTPPVLDSDGSFFLYSKLTVDKSRWQQGNVFSCSVMHEALHNHYTQKSLSLSPGKGGGGG | |
| GSPDRPWNPPTFSPALLVVTEGDNATFTCSFSNTSESFVLNWYRMSPSNQTDKLAAFPEDRSQPGQDSRFR | |
| VTQLPNGRDFHMSVVRARRNDSGTYLCGAISLAPKAQIKESLRAELRVTEP | |
| CL: NKG2D, CL | 19 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGGGGGRTVAAPS | |
| VFIFPPSDEQLKSGTASVVCLLNNFYPREAKVQWKVDNALQSGNSQESVTEQDSKDSTYSLSSTLTLSKAD | |
| YEKQKVYACEVTHQGLSSPVTKSFLRGEC | |
| CH1: NKG2D, MCH1wt, linker-hinge, CH2—CH3 (hole), TREM2 | 20 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGGGGGGSASAPT | |
| LFPLVSCENSPSDTSSVAVGCLAQDFLPDSITFSWKYKNNSDISSTRGFPSVLRGGKYAATSQVLLPSKDVM | |
| QGTDEHVVCKVQHPNGNKEKNVPLPAGGGGGGTHTCPPCPAPEAAGGPSVFLFPPKPKDTLMISRTPEVTC | |
| VVVDVSHEDPEVKFNWYVDGVEVHNAKTKPREEQYNSTYRVVSVLTVLHQDWLNGKEYKCKVSNKALP | |
| APIEKTISKAKGQPREPQVCTLPPSRDELTKNQVSLSCAVKGFYPSDIAVEWESNGQPENNYKTTPPVLDSD | |
| GSFFLVSKLTVDKSRWQQGNVFSCSVMHEALHNHYTQKSLSLSPGKGGGGGGHNTTVFQGVAGQSLQVS | |
| CPYDSMKHWGRRKAWCRQLGEKGPCQRVVSTHNLWLLSFLRRWNGSTAITDDTLGGTLTITLRNLQPHD | |
| AGLYQCQSLHGSEADTLRKVLVEVLA | |
| Linker: TIM3, linker-hinge, CH2—CH3 (knob)-PD-1 | 21 |
| SEVEYRAEVGQNAYLPCFYTPAAPGNLVPVCWGKGACPVFECGNVVLRTDERDVNYWTSRYWLNGDFR | |

KGDVSLTIENVTLADSGIYCCRIQIPGIMNDEKFNLKLVIKPAGGGGGGTHTCPPCPAPEAAGGPSVFLFPPK

PKDTLMISRTPEVTCVVVDVSHEDPEVKFNWYVDGVEVHNAKTKPREEQYNSTYRVVSVLTVLHQDWLN

GKEYKCKVSNKALPAPIEKTISKAKGQPREPQVYTLPPCRDELTKNQVSLWCLVKGFYPSDIAVEWESNGQ

| | |
|---|---|
| PENNYKTTPPVLDSDGSFFLYSKLTVDKSRWQQGNVFSCSVMHEALHNHYTQKSLSLSPGKGGGGGGSPD | |
| RPWNPPTFSPALLVVTEGDNATFTCSFSNTSESFVLNWYRMSPSNQTDKLAAFPEDRSQPGQDSRFRVTQLP | |
| NGRDFHMSVVRARRNDSGTYLCGAISLAPKAQIKESLRAELRVTEP | |
| CL: RS7Vk, CL | 22 |
| DIQLTQSPSSLSASVGDRVSITCKASQDVSIAVAWYQQKPGKAPKLLIYSASYRYTGVPDRFSGSGSGTDFT | |
| LTISSLQPEDFAVYYCQQHYITPLTFGAGTKVEIKRTVAAPSVFIFPPSDEQLKSGTASVVCLLNNFYPREAK | |
| VQWKVDNALQSGNSQESVTEQDSKDSTYSLSSTLTLSKADYEKQKVYACEVTHQGLSSPVTKSFLRGEC | |
| CH1: RS7Vh, MCH1wt, linker-hinge, CH2—CH3 (hole; LL) | 23 |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGSASAPTLFPLVSCENSPS

DTSSVAVGCLAQDFLPDSITFSWKYKNNSDISSTRGFPSVLRGGKYAATSQVLLPSKDVMQGTDEHVVCKV
QHPNGNKEKNVPLPAGGGGGGTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG

QPREPQVCTLPPSRDELTKNQVSLSCAVKGFYPSDIAVEWESNGQPENNYKTTPPVLDSDGSFFLVSKLTVD

| | |
|---|---|
| KSRWQQGNVFSCSVMHEALHNHYTQKSLSLSPGK | |
| Linker: TIM3, linker-hinge, CH2—CH3 (knob, LL), PD-1 | 24 |
| SEVEYRAEVGQNAYLPCFYTPAAPGNLVPVCWGKGACPVFECGNVVLRTDERDVNYWTSRYWLNGDFR | |
| KGDVSLTIENVTLADSGIYCCRIQIPGIMNDEKFNLKLVIKPAGGGGGGTHTCPPCPAPELLGGPSVFLFPPKP | |
| KDTLMISRTPEVTCVVVDVSHEDPEVKFNWYVDGVEVHNAKTKPREEQYNSTYRVVSVLTVLHQDWLNG | |

KEYKCKVSNKALPAPIEKTISKAKGQPREPQVYTLPPCRDELTKNQVSLWCLVKGFYPSDIAVEWESNGQP

| | |
|---|---|
| ENNYKTTPPVLDSDGSFFLYSKLTVDKSRWQQGNVFSCSVMHEALHNHYTQKSLSLSPGKGGGGGGSPDR | |
| PWNPPTFSPALLVVTEGDNATFTCSFSNTSESFVLNWYRMSPSNQTDKLAAFPEDRSQPGQDSRFRVTQLP | |
| NGRDFHMSVVRARRNDSGTYLCGAISLAPKAQIKESLRAELRVTEP | |
| CH1: Vd, MCH1wt, linker-hinge, CH2—CH3 (hole), PD-1 | 25 |
| QKVTQAQSSVSMPVRKAVTLNCLYETSWWSYYIFWYKQLPSKEMIFLIRQGSDEQNAKSGRYSVNFKKAA | |
| KSVALTISALQLEDSAKYFCALGVRAFLRDWGIRVLIFGKGTRVTVEPGGGGGGSASAPTLFPLVSCENSPS | |
| DTSSVAVGCLAQDFLPDSITFSWKYKNNSDISSTRGFPSVLRGGKYAATSQVLLPSKDVMQGTDEHVVCKV | |

QHPNGNKEKNVPLPAGGGGGGTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKG
QPREPQVCTLPPSRDELTKNQVSLSCAVKGFYPSDIAVEWESNGQPENNYKTTPPVLDSDGSFFLVSKLTVD
KSRWQQGNVFSCSVMHEALHNHYTQKSLSLSPGKGGGGGGSPDRPWNPPTFSPALLVVTEGDNATFTCSF
SNTSESFVLNWYRMSPSNQTDKLAAFPEDRSQPGQDSRFRVTQLPNGRDFHMSVVRARRNDSGTYLCGAI

| | |
|---|---|
| SLAPKAQIKESLRAELRVTEP | |
| CH1(2): SP34Vh, MCH1wt, linker-hinge, CH2—CH3 (knob), TIM3 | 26 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGSASAPTLFPLVS

CENSPSDTSSVAVGCLAQDFLPDSITFSWKYKNNSDISSTRGFPSVLRGGKYAATSQVLLPSKDVMQGTDE
HVVCKVQHPNGNKEKNVPLPGGPAGGGGTHTCPPCPAPEAAGGPSVFLFPPKPKDTLMISRTPEVTCVVVD

VSHEDPEVKFNWYVDGVEVHNAKTKPREEQYNSTYRVVSVLTVLHQDWLNGKEYKCKVSNKALPAPIEK
TISKAKGQPREPQVYTLPPCRDELTKNQVSLWCLVKGFYPSDIAVEWESNGQPENNYKTTPPVLDSDGSFF

| | |
|---|---|
| LYSKLTVDKSRWQQGNVFSCSVMHEALHNHYTQKSLSLSPGGGGGGSSEVEYRAEVGQNAYLPCFYTPA | |
| APGNLVPVCWGKGACPVFECGNVVLRTDERDVNYWTSRYWLNGDFRKGDVSLTIENVTLADSGIYCCRI | |
| QIPGIMNDEKFNLKLVIKP | |
| A1: NKG2D, linker-hinge, CH2, CH3 (hole), linker, CH3 (knob), PD-1 | 27 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGGPAGGGGTHTC | |
| PPCPAPEAAGGPSVFLFPPKPKDTLMISRTPEVTCVVVDVSHEDPEVKFNWYVDGVEVHNAKTKPREEQYN | |
| STYRVVSVLTVLHQDWLNGKEYKCKVSNKALPAPIEKTISKAKGQPREPQVCTLPPSRDELTKNQVSLSCA | |

VKGFYPSDIAVEWESNGQPENNYKTTPPVLDSDGSFFLVSKLTVDKSRWQQGNVFSCSVMHEALHNHYTQ
KSLSLSPGGGGGGGQPREPQVYTLPPCRDELTKNQVSLWCLVKGFYPSDIAVEWESNGQPENNYKTTPPVL

DSDGSFFLYSKLTVDKSRWQQGNVFSCSVMHEALHNHYTQKSLSLSPGKGGGGGGSPDRPWNPPTFSPAL

LVVTEGDNATFTCSFSNTSESFVLNWYRMSPSNQTDKLAAFPEDRSQPGQDSRFRVTQLPNGRDFHMSVV

| | |
|---|---|
| RARRNDSGTYLCGAISLAPKAQIKESLRAELRVTEP | |
| A2: NKG2D, linker-hinge, CH2, CH3 (knob), linker, CH3 (hole), linker, | 28 |
| DR5scFv | |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGGPAGGGGGGT | |
| HTCPPCPAPEAAGGPSVFLFPPKPKDTLMISRTPEVTCVVVDVSHEDPEVKFNWYVDGVEVHNAKTKPREE | |
| QYNSTYRVVSVLTVLHQDWLNGKEYKCKVSNKALPAPIEKTISKAKGQPREPQVYTLPPCRDELTKNQVS | |
| LWCLVKGFYPSDIAVEWESNGQPENNYKTTPPVLDSDGSFFLYSKLTVDKSRWQQGNVFSCSVMHEALHN | |

HYTQKSLSLSPGGGGGGGQPREPQVCTLPPSRDELTKNQVSLSCAVKGFYPSDIAVEWESNGQPENNYKTT
PPVLDSDGSFFLVSKLTVDKSRWQQGNVFSCSVMHEALHNHYTQKSLSLSPGKGGGGGGEVQLVQSGGG

VERPGGSLRLSCAASGFTFDDYGMSWVRQAPGKGLEWVSGINWNGGSTGYADSVKGRVTISRDNAKNSL

YLQMNSLRAEDTAVYYCAKILGAGRGWYFDLWGKGTTVTVSSGGGGSGGGGSGGGGSSSELTQDPAVSV
ALGQTVRITCQGDSLRSYYASWYQQKPGQAPVLVIYGKNNRPSGIPDRFSGSSSGNTASLTITGAQAEDEA

| | |
|---|---|
| DYYCNSRDSSGNHVVFGGGTKLTVL | |
| B1: SP34Vk, linker, CH3 (knob), linker, CH2, CH3 (hole), PD-1 | 29 |
| QAVVTQEPSLTVSPGGTVTLTCGSSTGA VTTSNYANWVQQKPGQAFRGLIGGTNKRAPGTPARFSGSLLGG | |
| KAALTLSGAQPEDEAEYYCALWYSNLWVFGGGTKLTVLGGGGGGQPREPQVYTLPPCRDELTKNQVSLW | |
| CLVKGFYPSDIAVEWESNGQPENNYKTTPPVLDSDGSFFLYSKLTVDKSRWQQGNVFSCSVMHEALHNHY | |
| TQKSLSLSPGGGGGGPSVFLFPPKPKDTLMISRTPEVTCVVVDVSHEDPEVKFNWYVDGVEVHNAKTKPRE | |
| EQYNSTYRVVSVLTVLHQDWLNGKEYKCKVSNKALPAPIEKTISKAKGQPREPQVCTLPPSRDELTKNQVS | |

LSCAVKGFYPSDIAVEWESNGQPENNYKTTPPVLDSDGSFFLVSKLTVDKSRWQQGNVFSCSVMHEALHN

| | |
|---|---|
| HYTQKSLSLSPGKGGGGGGSPDRPWNPPTFSPALLVVTEGDNATFTCSFSNTSESFVLNWYRMSPSNQTDK | |
| LAAFPEDRSQPGQDSRFRVTQLPNGRDFHMSVVRARRNDSGTYLCGAISLAPKAQIKESLRAELRVTEP | |
| B1: SP34Vh, linker, CH3 (hole), CH2, CH3 (knob), linker, RS7scFv | 30 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGGGGGGQPREPQ
VCTLPPSRDELTKNQVSLSCAVKGFYPSDIAVEWESNGQPENNYKTTPPVLDSDGSFFLVSKLTVDKSRWQ

QGNVFSCSVMHEALHNHYTQKSLSLSPGGGGGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPR

EPQVYTLPPCRDELTKNQVSLWCLVKGFYPSDIAVEWESNGQPENNYKTTPPVLDSDGSFFLYSKLTVDKS

RWQQGNVFSCSVMHEALHNHYTQKSLSLSPGKGGGGGQVQLGQSGSELKKPGASVKVSCKASGYTFTSY

AMNWVRQAPGQGLKWMGWINTYTGETPYTDDFKGRFAFSLDTSVSTAYLQISSLKADDTAVYFCARGGF
GSSYWYFDVWGQGTLVTVSSGGGGSGGGGSGGGGSDIQLTQSPSSLSASVGDRVSITCKASQDVSIAVAW

| | |
|---|---|
| YQQKPGKAPKLLIYSASYRYTGVPDRFSGSGSGTDFTLTISSLQPEDFAVYYCQQHYITPLTFGAGTKVEIK | |
| C1: IL15a, linker, CH3 (knob), linker-hinge (LL), CH2, CH3 (hole), linker, | 31 |
| b2GP1-D5 | |
| QITCPPPMSVEHADIWVKSYSLYSRERYICNSGFKRKAGTSSLTECVLNKATNVAHWTTPSLKCIRDPALGG | |
| GGGQPREPQVYTLPPCRDELTKNQVSLWCLVKGFYPSDIAVEWESNGQPENNYKTTPPVLDSDGSFFLYSK | |

LTVDKSRWQQGNVFSCSVMHEALHNHYTQKSLSLSPGGAPGGGTHTCPPCPAPELLGGPSVFLFPPKPKDT
LMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKSLSLSPGKGGGGGGASCKVPVK
KATVVYQGERVKIQEKFKNGMLHGDK VSFFCKNKEKKCSYTEDAQCIDGTIEVPKCFKEHSSLAFWKTDA

| | |
|---|---|
| SDVKPC | |
| C2: IL15, linker, CH3 (hole), linker-hinge (LL), CH2, CH3 (knob), linker, | 32 |
| RS7scFv | |
| NWVNVISDLKKIEDLIQSMHIDATLYTESDVHPSCKVTAMKCFLLELQVISLESGDASIHDTVENLIILANNS | |
| LSSNGNVTESGCKECEELEEKNIKEFLQSFVHIVQMFINGGGGGGQPREPQVCTLPPSRDELTKNQVSLSCA | |

VKGFYPSDIAVEWESNGQPENNYKTTPPVLDSDGSFFLVSKLTVDKSRWQQGNVFSCSVMHEALHNHYTQ

KSLSLSPGGAPGGGTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYT

LPPCRDELTKNQVSLWCLVKGFYPSDIAVEWESNGQPENNYKTTPPVLDSDGSFFLYSKLTVDKSRWQQG

NVFSCSVMHEALHNHYTQKSLSLSPGKGGGGGQVQLGQSGSELKKPGASVKVSCKASGYTFTSYAMNWV

RQAPGQGLKWMGWINTYTGETPYTDDFKGRFAFSLDTSVSTAYLQISSLKADDTAVYFCARGGFGSSYWY

| | |
|---|---|
| FDVWGQGTLVTVSSGGGGSGGGGSGGGGSDIQLTQSPSSLSASVGDRVSITCKASQDVSIAVAWYQQKPG | |
| KAPKLLIYSASYRYTGVPDRFSGSGSGTDFTLTISSLQPEDFAVYYCQQHYITPLTFGAGTKVEIK | |
| CL: RS7Vk, CL, linker, CD8 stalk & tm, P2A, signal, PD-1 tm decoy | 33 |
| DIQLTQSPSSLSASVGDRVSITCKASQDVSIAVAWYQQKPGKAPKLLIYSASYRYTGVPDRFSGSGSGTDFT | |
| LTISSLQPEDFAVYYCQQHYITPLTFGAGTKVEIKRTVAAPSVFIFPPSDEQLKSGTASVVCLLNNFYPREAK | |
| VQWKVDNALQSGNSQESVTEQDSKDSTYSLSSTLTLSKADYEKQKVYACEVTHQGLSSPVTKSFLRGECG | |

GPAGPTTTPAPRPPTPAPTIASQPLSLRPEACRPAAGGAVHTRGLDFACDIYIWAPLAGTCGVLLLSLVITLY
CKRGRAGSGATNFSLLKQAGDVEENPGPMQIPQAPWPVVWAVLQLGWRPGWFLDSPDRPWNPPTFSPAL

| | |
|---|---|
| LVVTEGDNATFTCSFSNTSESFVLNWYRMSPSNQTDKLAAFPEDRSQPGQDCRFRVTQLPNGRDFHMSVV | |
| RARRNDSGTYLCGAISLAPKAQIKESLRAELRVTERRAEVPTAHPSPSPRPAGQFQTLVVGVVGGLLGSLVL | |
| LVWVLAVICSRAP | |
| CH1: RS7Vh, MCH1wt, CD8 stalk & tm, CD137, CD3z | 34 |
| QVQLGQSGSELKKPGASVKVSCKASGYTFTSYAMNWVRQAPGQGLKWMGWINTYTGETPYTDDFKGRF | |
| AFSLDTSVSTAYLQISSLKADDTAVYFCARGGFGSSYWYFDVWGQGTLVTVSSGSASAPTLFPLVSCENSPS | |
| DTSSVAVGCLAQDFLPDSITFSWKYKNNSDISSTRGFPSVLRGGKYAATSQVLLPSKDVMQGTDEHVVCKV | |
| QHPNGNKEKNVPLGGPAGPTTTPAPRPPTPAPTIASQPLSLRPEACRPAAGGAVHTRGLDFACDIYIWAPLA | |

GTCGVLLLSLVITLYCKRGRKKLLYIFKQPFMRPVQTTQEEDGCSCRFPEEEEGGCELRVKFSRSADAPAYQ
QGQNQLYNELNLGRREEYDVLDKRRGRDPEMGGKPQRRKNPQEGLYNELQKDKMAEAYSEIGMKGERR

| | |
|---|---|
| RGKGHDGLYQGLSTATKDTYDALHMQALPPR | |
| CL: RS7Vk, linker, CL, CD8 stalk & tm, P2A, CD137Ltm | 35 |
| DIQLTQSPSSLSASVGDRVSITCKASQDVSIAVAWYQQKPGKAPKLLIYSASYRYTGVPDRFSGSGSGTDFT | |
| LTISSLQPEDFAVYYCQQHYITPLTFGAGTKVEIKRTVAAPSVFIFPPSDEQLKSGTASVVCLLNNFYPREAK | |
| VQWKVDNALQSGNSQESVTEQDSKDSTYSLSSTLTLSKADYEKQKVYACEVTHQGLSSPVTKSFLRGECG | |

GPAGPTTTPAPRPPTPAPTIASQPLSLRPEACRPAAGGAVHTRGLDFACDIYIWAPLAGTCGVLLLSLVITLY

| | |
|---|---|
| CKRGRAGSGATNFSLLKQAGDVEENPGPMEYASDASLDPEAPWPPAPRARACRVLPWALVAGLLLLLLLA | |
| AACAVFLACPWAVSGARASPGSAASPRLREGPELSPDDPAGLLDLRQGMFAQLVAQNVLLIDGPLSWYSD | |
| PGLAGVSLTGGLSYKEDTKELVVAKAGVYYVFFQLELRRVVAGEGSGSVSLALHLQPLRSAAGAAALALT | |
| VDLPPASSEARNSAFGFQGRLLHLSAGQRLGVHLHTEARARHAWQLTQGATVLGLFRVTP | |
| CH1: RS7Vh, linker, MCH1wt, CD8 stalk & tm, CD137, CD3z, PA2, PD- | 36 |
| 1tm decoy | |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGSASAPTLFPLVSCENSPS

DTSSVAVGCLAQDFLPDSITFSWKYKNNSDISSTRGFPSVLRGGKYAATSQVLLPSKDVMQGTDEHVVCKV
QHPNGNKEKNVPLGGPAGPTTTPAPRPPTPAPTIASQPLSLRPEACRPAAGGAVHTRGLDFACDIYIWAPLA
GTCGVLLLSLVITLYCRVKFSRSADAPAYQQGQNQLYNELNLGRREEYDVLDKRRGRDPEMGGKPQRRK
NPQEGLYNELQKDKMAEAYSEIGMKGERRRGKGHDGLYQGLSTATKDTYDALHMQALPPRGSGATNFSL

LKQAGDVEENPGPMQIPQAPWPVVWAVLQLGWRPGWFLDSPDRPWNPPTFSPALLVVTEGDNATFTCSFS

| | |
|---|---|
| NTSESFVLNWYRMSPSNQTDKLAAFPEDRSQPGQDCRFRVTQLPNGRDFHMSVVRARRNDSGTYLCGAIS | |
| LAPKAQIKESLRAELRVTERRAEVPTAHPSPSPRPAGQFQTLVVGVVGGLLGSLVLLVWVLAVICSRAP | |
| CL: RS7scFv, linker, CL, CD8 stalk & tm, PA2, CD137L | 37 |
| QVQLGQSGSELKKPGASVKVSCKASGYTFTSYAMNWVRQAPGQGLKWMGWINTYTGETPYTDDFKGRF | |
| AFSLDTSVSTAYLQISSLKADDTAVYFCARGGFGSSYWYFDVWGQGTLVTVSSGGGGSGGGGSGGGGSDI | |

QLTQSPSSLSASVGDRVSITCKASQDVSIAVAWYQQKPGKAPKLLIYSASYRYTGVPDRFSGSGSGTDFTLTI

SSLQPEDFA VYYCQQHYITPLTFGAGTKVEIKGGGGGRTVAAPSVFIFPPSDEQLKSGTASVVCLLNNFYPR
EAKVQWKVDNALQSGNSQESVTEQDSKDSTYSLSSTLTLSKADYEKQKVYACEVTHQGLSSPVTKSFLRG

ECGGPAGPTTTPAPRPPTPAPTIASQPLSLRPEACRPAAGGAVHTRGLDFACDIYIWAPLAGTCGVLLLSLVI

| | |
|---|---|
| TLYCKRGRAGSGATNFSLLKQAGDVEENPGPMEYASDASLDPEAPWPPAPRARACRVLPWALVAGLLLLL | |
| LLAAACAVFLACPWAVSGARASPGSAASPRLREGPELSPDDPAGLLDLRQGMFAQLVAQNVLLIDGPLSW | |
| YSDPGLAGVSLTGGLSYKEDTKELVVAKAGVYYVFFQLELRRVVAGEGSGSVSLALHLQPLRSAAGAAAL | |
| ALTVDLPPASSEARNSAFGFQGRLLHLSAGQRLGVHLHTEARARHAWQLTQGATVLGLFRVTP | |
| CH1: TIM3, linker, MCH1wt, CD8 stalk & tm, CD137, CD3z | 38 |
| SEVEYRAEVGQNAYLPCFYTPAAPGNLVPVCWGKGACPVFECGNVVLRTDERDVNYWTSRYWLNGDFR | |

KGDVSLTIENVTLADSGIYCCRIQIPGIMNDEKFNLKLVIKPGGGGGGSASAPTLFPLVSCENSPSDTSSVAV

GCLAQDFLPDSITFSWKYKNNSDISSTRGFPSVLRGGKYAATSQVLLPSKDVMQGTDEHVVCKVQHPNGN

KEKNVPLGGPAGPTTTPAPRPPTPAPTIASQPLSLRPEACRPAAGGAVHTRGLDFACDIYIWAPLAGTCGVL

| | |
|---|---|
| LLSLVITLYCKRGRKKLLYIFKQPFMRPVQTTQEEDGCSCRFPEEEEGGCELRVKFSRSADAPAYQQGQNQ | |
| LYNELNLGRREEYDVLDKRRGRDPEMGGKPQRRKNPQEGLYNELQKDKMAEAYSEIGMKGERRRGKGH | |
| DGLYQGLSTATKDTYDALHMQALPPR | |
| CL: Vg, CL, CD8 stalk, CD8tm, PA2, TIGIT tm (decoy) | 39 |
| HLEQPQISSTKTLSKTARLECVVSGITISATSVYWYRERPGEVIQFLVSISYDGTVRKESGIPSGKFEVDRIPET | |
| STSTLTIHNVEKQDIATYYCALWDRYYKKLFGSGTTLVVTDGGGGGRTVAAPSVFIFPPSDEQLKSGTASV | |
| VCLLNNFYPREAKVQWKVDNALQSGNSQESVTEQDSKDSTYSLSSTLTLSKADYEKHKVYACEVTHQGLS | |

SPVTKSFNRGECGGPAGPTTTPAPRPPTPAPTIASQPLSLRPEACRPAAGGAVHTRGLDFACDIYIWAPLAGT

CGVLLLSL VITLYCKRGRAGSGATNFSLLKQAGDVEENPGPMRWCLLLIWAQGLRQAPLASGMMTGTIET
TGNISAEKGGSIILQCHLSSTTAQVTQVNWEQQDQLLAICNADLGWHISPSFKDRVAPGPGLGLTLQSLTVN

DTGEYFCIYHTYPDGTYTGRIFLEVLESSVAEHGARFQIPLLGAMAATLVVICTAVIVVVALTRKK

| | |
|---|---|
| CH1: Vd, MCH1, CD8 stalk, CD8tm, CD137, CD3z, PA2, CD137L | 40 |
| QKVTQAQSSVSMPVRKAVTLNCLYETSWWSYYIFWYKQLPSKEMIFLIRQGSDEQNAKSGRYSVNFKKAA | |

KSVALTISALQLEDSAKYFCALGVRAFLRDWGIRVLIFGKGTRVTVEPGGGGGGSASAPTLFPL VSCENSPS

DTSSVAVGCLAQDFLPDSITFSWKYKNNSDISSTRGFPSVLRGGKYAATSQVLLPSKDVMQGTDEHVVCKV

QHPNGNKEKNVPLGGPAGPTTTPAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELRVKFSRSADAPAYQ

QGQNQLYNELNLGRREEYDVLDKRRGRDPEMGGKPQRRKNPQEGLYNELQKDKMAEAYSEIGMKGERR
RGKGHDGLYQGLSTATKDTYDALHMQALPPRGSGATNFSLLKQAGDVEENPGPMEYASDASLDPEAPWP

PAPRARACRVLPWALVAGLLLLLLLAAACAVFLACPWAVSGARASPGSAASPRLREGPELSPDDPAGLLD

| | |
|---|---|
| LRQGMFAQL VAQNVLLIDGPLSWYSDPGLAGVSLTGGLSYKEDTKELVVAKAGVYYVFFQLELRRVVAG | |
| EGSGSVSLALHLQPLRSAAGAAALALTVDLPPASSEARNSAFGFQGRLLHLSAGQRLGVHLHTEARARHA | |
| WQLTQGATVLGLFRVTP | |
| CL: Vg, CL, CD8-stalk & tm, CD137, CD3z, PA2 CXCL10 | 41 |

HLEQPQISSTKTLSKTARLECVVSGITISATSVYWYRERPGEVIQFLVSISYDGTVRKESGIPSGKFEVDRIPET

STSTLTIHNVEKQDIATYYCALWDRYYKKLFGSGTTLVVTDGGGGGRTVAAPSVFIFPPSDEQLKSGTASV
VCLLNNFYPREAKVQWKVDNALQSGNSQESVTEQDSKDSTYSLSSTLTLSKADYEKQKVYACEVTHQGLS

SPVTKSFLRGECGGPAGPTTTPAPRPPTPAPTIASQPLSLRPEACRPAAGGAVHTRGLDFACDIYIWAPLAGT
CGVLLLSLVITLYCKRGRAGSGATNFSLLKQAGDVEENPGPMEFGLSWVFLVALLRGVQCAPARSPSPSTQ

| | |
|---|---|
| PWEHVNAIQEARRLLNLSRDTAAEMNETVEVISEMFDLQEPTCLQTRLELYKQGLRGSLTKLKGPLTMMA | |
| SHYKQHCPPTPETSCATQIITFESFKENLKDFLLVIPFDCWEPVQE | |
| CH1: Vd, MCH1wt, CD8-stalk & tm, CD18, PA2, PD-1 tm (decoy) | 42 |
| QKVTQAQSSVSMPVRKAVTLNCLYETSWWSYYIFWYKQLPSKEMIFLIRQGSDEQNAKSGRYSVNFKKAA | |

KSVALTISALQLEDSAKYFCALGVRAFLRDWGIRVLIFGKGTRVTVEPGGGGGGSASAPTLFPLVSCENSPS

DTSSVAVGCLAQDFLPDSITFSWKYKNNSDISSTRGFPSVLRGGKYAATSQVLLPSKDVMQGTDEHVVCKV

QHPNGNKEKNVPLGGPAGPTTTPAPRPPTPAPTIASQPLSLRPEACRPAAGGAVHTRGLDFACDIYIWAPLA

GTCGVLLLSLVITLYCKALIHLSDLREYRRFEKEKLKSQWNNDNPLFKSATTTVMNPKFAESGSGATNFSLL

KQAGDVEENPGPMEYASDASLDPEAPWPPAPRARACRVLPWALVAGLLLLLLLAAACAVFLACPWAVSG

| | |
|---|---|
| ARASPGSAASPRLREGPELSPDDPAGLLDLRQGMFAQLVAQNVLLIDGPLSWYSDPGLAGVSLTGGLSYKE | |
| DTKELVVAKAGVYYVFFQLELRRVVAGEGSGSVSLALHLQPLRSAAGAAALALTVDLPPASSEARNSAFG | |
| FQGRLLHLSAGQRLGVHLHTEARARHAWQLTQGATVLGLFRVTP | |
| CL: hRS7Vk, CL, CD8-stalk & tm, CD11b, CD3z | 43 |
| DIQLTQSPSSLSASVGDRVSITCKASQDVSIAVAWYQQKPGKAPKLLIYSASYRYTGVPDRFSGSGSGTDFT | |

LTISSLQPEDFAVYYCQQHYITPLTFGAGTKVEIKRTVAAPSVFIFPPSDEQLKSGTASVVCLLNNFYPREAK

VQWKVDNALQSGNSQESVTEQDSKDSTYSLSSTLTLSKADYEKQKVYACEVTHQGLSSPVTKSFLRGECG

GPAGPTTTPAPRPPTPAPTIASQPLSLRPEACRPAAGGAVHTRGLDFACDIYIWAPLAGTCGVLLLSLVITLY
CKLGFFKRQYKDMMSEGGPPGAEPQGGSVKFSRSADAPAYQQGQNQLYNELNLGRREEYDVLDKRRGRD
PEMGGKPQRRKNPQEGLYNELQKDKAEAYSEIGMKGERRRGKGHDGLYQGLSTATKDTYDALHMQALP

| | |
|---|---|
| PR | |
| CH1: hRS7Vh, MCH1, CD8-stalk & tm, CD18, CD137 | 44 |
| QVQLGQSGSELKKPGASVKVSCKASGYTFTSYAMNWVRQAPGQGLKWMGWINTYTGETPYTDDFKGRF | |
| AFSLDTSVSTAYLQISSLKADDTAVYFCARGGFGSSYWYFDVWGQGTLVTVSSGSASAPTLFPLVSCENSPS | |
| DTSSVAVGCLAQDFLPDSITFSWKYKNNSDISSTRGFPSVLRGGKYAATSQVLLPSKDVMQGTDEHVVCKV | |
| QHPNGNKEKNVPLGGPAGPTTTPAPRPPTPAPTIASQPLSLRPEACRPAAGGAVHTRGLDFACDIYIWAPLA | |
| GTCGVLLLSLVITLYCKALIHLSDLREYRRFEKEKLKSQWNNDNPLFKSATTTVMNPKFAESGGSRGRKKL | |
| LYIFKQPFMRPVQTTQEEDGCSCRFPEEEEGGCEL | |
| CL: hRS7Vk, CL, CD8-stalk & tm, CD137, CD3, PA2, CXCL10 | 45 |
| DIQLTQSPSSLSASVGDRVSITCKASQDVSIAVAWYQQKPGKAPKLLIYSASYRYTGVPDRFSGSGSGTDFT | |

LTISSLQPEDFAVYYCQQHYITPLTFGAGTKVEIKRTVAAPSVFIFPPSDEQLKSGTASVVCLLNNFYPREAK

VQWKVDNALQSGNSQESVTEQDSKDSTYSLSSTLTLSKADYEKQKVYACEVTHQGLSSPVTKSFLRGECG

GPAGPTTTPAPRPPTPAPTIASQPLSLRPEACRPAAGGAVHTRGLDFACDIYIWAPLAGTCGVLLLSLVITLY

CKRGRKKLLYIFKQPFMRPVQTTQEEDGCSCRFPEEEEGGCELRVKFSRSADAPAYQQGQNQLYNELNLG
RREEYDVLDKRRGRDPEMGGKPQRRKNPQEGLYNELQKDKMAEAYSEIGMKGERRRGKGHDGLYQGLS

TATKDTYDALHMQALPPRGSGATNFSLLKQAGDVEENPGPMEFGLSWVFLVALLRGVQCAPARSPSPSTQ

| | |
|---|---|
| PWEHVNAIQEARRLLNLSRDTAAEMNETVEVISEMFDLQEPTCLQTRLELYKQGLRGSLTKLKGPLTMMA | |
| SHYKQHCPPTPETSCATQIITFESFKENLKDFLL VIPFDCWEPVQE | |
| CH1: hRS7Vh, MCH1, CD8-stalk & tm, LRPIt | 46 |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGSASAPTLFPLVSCENSPS
DTSSVAVGCLAQDFLPDSITFSWKYKNNSDISSTRGFPSVLRGGKYAATSQVLLPSKDVMQGTDEHVVCKV
QHPNGNKEKNVPLGGPAGPTTTPAPRPPTPAPTIASQPLSLRPEACRPAAGGAVHTRGLDFACDIYIWAPLA

| | |
|---|---|
| GTCGVLLLSLVITLYCKRRVQGAKGFQHQRMTNGAMNVEIGNPTYKMYEGGEPDDVGGLLDADFALDPD | |
| KPTNFTNPVYATLYMGGHGSRHSLASTDEKRELLGRGPEDEIGDPLA | |
| CL: TIM4, CL, CD8-stalk & tm, LRP1t | 47 |
| ETVVTEVLGHRVTLPCLYSSWSHNSNSMCWGKDQCPYSGCKEALIRTDGMRVTSRKSAKYRLQGTIPRGD | |
| VSLTILNPSESDSGVYCCRIEVPGWFNDVKINVRLNLQRAGGGGGRTVAAPSVFIFPPSDEQLKSGTASVVC | |
| LLNNFYPREAKVQWKVDNALQSGNSQESVTEQDSKDSTYSLSSTLTLSKADYEKQKVYACEVTHQGLSSP | |

VTKSFLRGECGGPAGPTTTPAPRPPTPAPTIASQPLSLRPEACRPAAGGAVHTRGLDFACDIYIWAPLAGTCG
VLLLSLVITLYCKRRVQGAKGFQHQRMTNGAMNVEIGNPTYKMYEGGEPDDVGGLLDADFALDPDKPTN

| | |
|---|---|
| FTNPVYATLYMGGHGSRHSLASTDEKRELLGRGPEDEIGDPLA | |
| CH1: RS7scFv, MCH1, CD8-stalk & tm, CD137, CD3z | 48 |
| QVQLGQSGSELKKPGASVKVSCKASGYTFTSYAMNWVRQAPGQGLKWMGWINTYTGETPYTDDFKGRF | |

AFSLDTSVSTAYLQISSLKADDTAVYFCARGGFGSSYWYFDVWGQGTLVTVSSGGGGSGGGGSGGGGSDI
QLTQSPSSLSASVGDRVSITCKASQDVSIAVAWYQQKPGKAPKLLIYSASYRYTGVPDRFSGSGSGTDFTLTI
SSLQPEDFAVYYCQQHYITPLTFGAGTKVEIKGGGGGGSASAPTLFPLVSCENSPSDTSSVAVGCLAQDFLP

| | |
|---|---|
| DSITFSWKYKNNSDISSTRGFPSVLRGGKYAATSQVLLPSKDVMQGTDEHVVCKVQHPNGNKEKNVPLGG | |
| PAGPTTTPAPRPPTPAPTIASQPLSLRPEACRPAAGGAVHTRGLDFACDIYIWAPLAGTCGVLLLSLVITLYC | |
| KRGRKKLLYIFKQPFMRPVQTTQEEDGCSCRFPEEEEGGCELRVKFSRSADAPAYQQGQNQLYNELNLGR | |
| REEYDVLDKRRGRDPEMGGKPQRRKNPQEGLYNELQKDKMAEAYSEIGMKGERRRGKGHDGLYQGLST | |
| ATKDTYDALHMQALPPR | |
| CL: TREM2, linker, TREM2, Linker, CL, CD8-stalk & tm | 49 |
| HNTTVFQGVAGQSLQVSCPYDSMKHWGRRKAWCRQLGEKGPCQRVVSTHNLWLLSFLRRWNGSTAITD | |

DTLGGTLTITLRNLQPHDAGLYQCQSLHGSEADTLRKVLVEVLAGGGGGHNTTVFQGVAGQSLQVSCPYD

SMKHWGRRKAWCRQLGEKGPCQRVVSTHNLWLLSFLRRWNGSTAITDDTLGGTLTITLRNLQPHDAGLY
QCQSLHGSEADTLRKVLVEVLAGAPGGGRTVAAPSVFIFPPSDEQLKSGTASVVCLLNNFYPREAKVQWK
VDNALQSGNSQESVTEQDSKDSTYSLSSTLTLSKADYEKQKVYACEVTHQGLSSPVTKSFLRGECGGPAGP

TTTPAPRPPTPAPTIASQPLSLRPEACRPAAGGAVHTRGLDFACDIYIWAPLAGTCGVLLLSLVITLYCKRR

| | |
|---|---|
| CH1: TREM2, linker, MCH1, CD8-stalk & tm, LRP1t | 50 |
| HNTTVFQGVAGQSLQVSCPYDSMKHWGRRKAWCRQLGEKGPCQRVVSTHNLWLLSFLRRWNGSTAITD | |

DTLGGTLTITLRNLQPHDAGLYQCQSLHGSEADTLRKVLVEVLAPAGGGGGSASAPTLFPLVSCENSPSDTS

| | |
|---|---|
| SVAVGCLAQDFLPDSITFSWKYKNNSDISSTRGFPSVLRGGKYAATSQVLLPSKDVMQGTDEHVVCKVQH | |
| PNGNKEKNVPLGGPAGPTTTPAPRPPTPAPTIASQPLSLRPEACRPAAGGAVHTRGLDFACDIYIWAPLAGT | |
| CGVLLLSL VITLYCKRR VQGAKGFQHQRMTNGAMNVEIGNPTYKMYEGGEPDDVGGLLDADFALDPDKP | |
| TNFTNPVYATLYMGGHGSRHSLASTDEKRELLGRGPEDEIGDPLA | |
| P01871.4 | 51 |
| GSASAPTLFPLVSCENSPSDTSSVAVGCLAQDFLPDSITFSWKYKNNSDISSTRGFPSVLRGGKYAATSQVLL | |
| PSKDVMQGTDEHVVCKVQHPNGNKEKNV | |
| 2AGJ | 52 |
| GSASAPTLFPLVSCENSSPSSTVAVGCLAQDFLPDSITFSWKYKNNSDISSTRGFPS | |
| VLRGGKYAATSQVLLPSKDVMQGTDEHVVCKVQHPNGNKEKDV | |
| 1DEE | 53 |
| GSASAPTLFPLVSCENSNPSSTVAVGCLAQDFLPDSITFSWKYKNNSDISSTRGFPSVLRGGKYAATSQVLLP | |
| SKDVAQGTNEHVVCKVQHPNGNKEKDV | |
| 2J6E | 54 |
| GSASAPTLFPLVSCDTSSVAVGCLAQDFLPDSITFSWKYKNNSDISSTRGFPSVL | |
| RGGKYAATSQVLLPSKDVMQGTDEHVVCKVQHPNGNKEKNV | |
| 1st 3′UTR | 55 |
| CTTTCTTGCTGTCCAATTTCTATTAAAGGTTCCTTTGTTCCGTAAGTCCAACTACTAAACTGGGGGATAT | |
| TATGAAGGGCCTTGAGCATCTGGATTCTGCCTAATAAAAAACATTTATTTTCATTGCAAT | |
| 20v8: L: hTVkb-CL-tIg hinge-CH2—CH3-linker-CD137L | 56 |

DIQLTQSPSSLSASVGDRVSITCKASQDVSIAVAWYQQKPGKAPKLLIYSASYRTTGVPDRFSGSGSGTDFTL
TISSLQPEDFAYYCQQHYITPLTFGAGTKVEIKRTVAAPSVFIFPPSDEQLKSGTASVVCLLNNFYPREAKV
QWKVDNALQSGNSQESVTEQDSKDSTYSLSSTLTLSKADYEKQKVYACEVTHQGLSSPVTKSFLRGECGG
PAGGGGGTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVCTLPPSRD
ELTKNQVSLSCAVKGFYPSDIAVEWESNGQPENNYKTTPPVLDSDGSFFLVSKLTVDKSRWQQGNVFSCSV
MHEALHNHYTQKSLSLSPGKGGGGGGMFAQLVAQNVLLIDGPLSWYSDPGLAGVSLTGGLSYKEDTKEL
VVAKAGVYYVFFQLELRRVVAGEGSGSVSLALHLQPLRSAAGAAALALTVDLPPASSEARNSAFGFQGRL

| | |
|---|---|
| LHLSAGQRLGVHLHTEARARHAWQLTQGATVLGLFRVTP | |
| 18v22: H: hTVhb-CH1-tIg hinge-CH2—CH3(knob) | 57 |
| QVQLGQSGSELKKPGASVKVSCKASGYTFTNYGMNWVKQAPGQGLKWMGWINTYTGEPTYTDDFKGRF | |

AFSLDTSVSTAYLQISSLKADDTAVYFCARGGFGSSYWYFDVWGQGSLVTVSSGSASAPTLFPL VSCENSPS

DTSSVAVGCLAQDFLPDSITFSWKYKNNSDISSTRGFPSVLRGGKYAATSQVLLPSKDVMQGTDEHVVCKV
QHPNGNKEKNVPLPGGPAGGGGGTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKA

KGQPREPQVYTLPPCRDELTKNQVSLWCLVKGFYPSDIAVEWESNGQPENNYKTTPPVLDSDGSFFLYSKL

| | |
|---|---|
| TVDKSRWQQGNVFSCSVMHEALHNHYTQKSLSLSPGK | |
| 18v26: L: hTVhb-CH1-tIg hinge-CH2-CH3(knob)-linker-CD137L domain | 58 |
| QVQLGQSGSELKKPGASVKVSCKASGYTFTNYGMNWVKQAPGQGLKWMGWINTYTGEPTYTDDFKGRF | |

AFSLDTSVSTAYLQISSLKADDTAVYFCARGGFGSSYWYFDVWGQGSLVTVSSGSASAPTLFPLVSCENSPS

DTSSVAVGCLAQDFLPDSITFSWKYKNNSDISSTRGFPSVLRGGKYAATSQVLLPSKDVMQGTDEHVVCKV
QHPNGNKEKNVPLPGGPAGGGGGTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKA

KGQPREPQVYTLPPCRDELTKNQVSLWCLVKGFYPSDIAVEWESNGQPENNYKTTPPVLDSDGSFFLYSKL

| | |
|---|---|
| TVDKSRWQQGNVFSCSVMHEALHNHYTQKSLSLSPGKGGGGGGMFAQLVAQNVLLIDGPLSWYSDPGLA | |
| GVSLTGGLSYKEDTKELVVAKAGVYYVFFQLELRRVVAGEGSGSVSLALHLQPLRSAAGAAALALTVDLP | |
| PASSEARNSAFGFQGRLLHLSAGQRLGVHLHTEARARHAWQLTQGATVLGLFRVTP | |
| L: 17v9: h3Vk-CL-link-tIg-hinge-G1Fc-linker-hTscFv | 59 |
| QAVVTQEPSLTVSPGGTVTLTCGSSTGAVTTSNYANWVQEKPGQAFRGLIGGTNKRAPGTPARFSGSLLGG | |
| KAALTLSGAQPEDEAEYYCALWYSNLWVFGCGTKLTVLRTVAAPSVFIFPPSDEQLKSGTASVVCLLNNFY | |
| PREAKVQWKVDNALQSGNSQESVTEQDSKDSTYSLSSTLTLSKADYEKHKVYACEVTHQGLSSPVTKSFN | |
| RGECGGPAGGGGGTHTCPPCPAPEAAGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISKAKGQPREPQVCT | |

LPPSRDELTKNQVSLSCAVKGFYPSDIAVEWESNGQPENNYKTTPPVLDSDGSFFLVSKLTVDKSRWQQGN

VFSCSVMHEALHNHYTQKSLSLSPGKGGGGGGQVQLGQSGSELKKPGASVKVSCKASGYTFTSYAMNWV

RQAPGQGLKWMGWINTYTGETPYTDDFKGRFAFSLDTSVSTAYLQISSLKADDTAVYFCARGGFGSSYWY

| | |
|---|---|
| FDVWGQGTLVTVSSGGGGSGGGGSGGGGSDIQLTQSPSSLSASVGDRVSITCKASQDVSIAVAWYQQKPG | |
| KAPKLLIYSASYRYTGVPDRFSGSGSGTDFTLTISSLQPEDFAVYYCQQHYITPLTFGAGTKVEIK | |
| H: 18: h3Vh-MCH1-linker-tIg hinge-G1Fc | 60 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGSASAPTLFPLVS

CENSPSDTSSVAVGCLAQDFLPDSITFSWKYKNNSDISSTRGFPSVLRGGKYAATSQVLLPSKDVMQGTDE
HVVCKVQHPNGNKEKNVPLPAGGGGGTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTI

SKAKGQPREPQVYTLPPCRDELTKNQVSLWCLVKGFYPSDIAVEWESNGQPENNYKTTPPVLDSDGSFFLY

| | |
|---|---|
| SKLTVDKSRWQQGNVFSCSVMHEALHNHYTQKSLSLSPGK | |
| L: 17v17: hTVkb-CL-linker-tIg hinge-G1Fc-linker-b2GP1-D5 | 61 |
| DIQLTQSPSSLSASVGDRVSITCKASQDVSIAVAWYQQKPGKAPKLLIYSASYRTTGVPDRFSGSGSGTDFTL | |
| TISSLQPEDFAVYYCQQHYITPLTFGAGTKVEIKRTVAAPSVFIFPPSDEQLKSGTASVVCLLNNFYPREAKV | |
| QWKVDNALQSGNSQESVTEQDSKDSTYSLSSTLTLSKADYEKQKVYACEVTHQGLSSPVTKSFLRGECGG | |
| PAGGGGGTHTCPPCPAPEAAGGPSVFLFPPKPKDTLMISRTPEVTCVVVDVSHEDPEVKFNWYVDGVEVH | |
| NAKTKPREEQYNSTYRVVSVLTVLHQDWLNGKEYKCKVSNKALPAPIEKTISKAKGQPREPQVCTLPPSRD | |

ELTKNQVSLSCAVKGFYPSDIAVEWESNGQPENNYKTTPPVLDSDGSFFLVSKLTVDKSRWQQGNVFSCSV

| | |
|---|---|
| MHEALHNHYTQKSLSLSPGKGGGGGGASCKVPVKKATVVYQGERVKIQEKFKNGMLHGDKVSFFCKNK | |
| EKKCSYTEDAQCIDGTIEVPKCFKEHSSLAFWKTDASDVKPC | |
| H: 18v23: hTVhb-MCH1-linker-tIg hinge-G1Fc-linker-h3scFv | 62 |
| QVQLGQSGSELKKPGASVKVSCKASGYTFTNYGMNWVKQAPGQGLKWMGWINTYTGEPTYTDDFKGRF | |

AFSLDTSVSTAYLQISSLKADDTAVYFCARGGFGSSYWYFDVWGQGSLVTVSSGSASAPTLFPLVSCENSPS

DTSSVAVGCLAQDFLPDSITFSWKYKNNSDISSTRGFPSVLRGGKYAATSQVLLPSKDVMQGTDEHVVCKV
QHPNGNKEKNVPLPGGPAGGGGGTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKA

KGQPREPQVYTLPPCRDELTKNQVSLWCLVKGFYPSDIAVEWESNGQPENNYKTTPPVLDSDGSFFLYSKL

| | |
|---|---|
| TVDKSRWQQGNVFSCSVMHEALHNHYTQKSLSLSPGKPAGGGGGSQAVVTQEPSLTVSPGGTVTLTCGSS | |
| TGAVTTSNYANWVQQKPGQAFRGLIGGTNKRAPGTPARFSGSLLGGKAALTLSGAQPEDEAEYYCALWY | |
| SNLWVFGGGTKLTVLGGGGSGGGGSGGGGSEVQLVESGGGLVQPGGSLKLSCAASGFTFNTYAMNWVR | |
| QAPGKGLEWVGRIRSKYNNYATYYADSVKGRFTISRDDSKNTLYLQMNSLKTEDTAVYYCVRHGNFGNS | |
| YVSWFAYWGQGTLVTVSS | |
| L: 17v7: h3Vk-CL-linker-tIg hinge-G1Fc-b2GP1-D5 | 63 |
| QAVVTQEPSLTVSPGGTVTLTCGSSTGAVTTSNYANWVQEKPGQAFRGLIGGTNKRAPGTPARFSGSLLGG | |
| KAALTLSGAQPEDEAEYYCALWYSNLWVFGCGTKLTVLRTVAAPSVFIFPPSDEQLKSGTASVVCLLNNFY | |
| PREAKVQWKVDNALQSGNSQESVTEQDSKDSTYSLSSTLTLSKADYEKHKVYACEVTHQGLSSPVTKSFN | |
| RGECGGPAGGGGGTHTCPPCPAPEAAGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISKAKGQPREPQVCT | |

LPPSRDELTKNQVSLSCAVKGFYPSDIAVEWESNGQPENNYKTTPPVLDSDGSFFLVSKLTVDKSRWQQGN

| | |
|---|---|
| VFSCSVMHEALHNHYTQKSLSLSPGKGGGGGGASCKVPVKKATVVYQGERVKIQEKFKNGMLHGDKVSF | |
| FCKNKEKKCSYTEDAQCIDGTIEVPKCFKEHSSLAFWKTDASDVKPC | |
| H: 18v9b: h3Vh-MCH1-linker-tIg hinge-G1Fc-b2GP1-D5 | 64 |
| EVQLVESGGGLVQPGGSLKLSCAASGFTFNTYAMNWVRQAPGKGLEWVGRIRSKYNNYATYYADSVKG | |
| RFTISRDDSKNTLYLQMNSLKTEDTAVYYCVRHGNFGNSYVSWFAYWGQGTLVTVSSGSASAPTLFPLVS | |
| CENSPSDTSSVAVGCLAQDFLPDSITFSWKYKNNSDISSTRGFPSVLRGGKYAATSQVLLPSKDVMQGTDE | |
| HVVCKVQHPNGNKEKNVPLPAGGGGGTHTCPPCPAPEAAGGPSVFLFPPKPKDTLMISRTPEVTCVVVDVS | |
| HEDPEVKFNWYVDGVEVHNAKTKPREEQYNSTYRVVSVLTVLHQDWLNGKEYKCKVSNKALPAPIEKTI | |

SKAKGQPREPQVYTLPPCRDELTKNQVSLWCLVKGFYPSDIAVEWESNGQPENNYKTTPPVLDSDGSFFLY

| | |
|---|---|
| SKLTVDKSRWQQGNVFSCSVMHEALHNHYTQKSLSLSPGKGGGGGGASCKVPVKKATVVYQGERVKIQE | |
| KFKNGMLHGDKVSFFCKNKEKKCSYTEDAQCIDGTIEVPKCFKEHSSLAFWKTDASDVKPC | |
| H: 18v25: hTVhb-MCH1-linker-tIg hinge-G1Fc-linker-b2GP1-D5 | 65 |
| QVQLGQSGSELKKPGASVKVSCKASGYTFTNYGMNWVKQAPGQGLKWMGWINTYTGEPTYTDDFKGRF | |

AFSLDTSVSTAYLQISSLKADDTAVYFCARGGFGSSYWYFDVWGQGSLVTVSSGSASAPTLFPLVSCENSPS

DTSSVAVGCLAQDFLPDSITFSWKYKNNSDISSTRGFPSVLRGGKYAATSQVLLPSKDVMQGTDEHVVCKV
QHPNGNKEKNVPLPGGPAGGGGGTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKA

KGQPREPQVYTLPPCRDELTKNQVSLWCLVKGFYPSDIAVEWESNGQPENNYKTTPPVLDSDGSFFLYSKL

| | |
|---|---|
| TVDKSRWQQGNVFSCSVMHEALHNHYTQKSLSLSPGKGGGGGGASCKVPVKKATVVYQGERVKIQEKFK | |
| NGMLHGDKVSFFCKNKEKKCSYTEDAQCIDGTIEVPKCFKEHSSLAFWKTDASDVKPC | |
| L: 17v19b: hTVkb-CL-linker-tIg hinge-G1Fc-linker-TRAIL domain | 66 |

DIQLTQSPSSLSASVGDRVSITCKASQDVSIAVAWYQQKPGKAPKLLIYSASYRTTGVPDRFSGSGSGTDFTL
TISSLQPEDFAVYYCQQHYITPLTFGAGTKVEIKRTVAAPSVFIFPPSDEQLKSGTASVVCLLNNFYPREAKV

QWKVDNALQSGNSQESVTEQDSKDSTYSLSSTLTLSKADYEKQKVYACEVTHQGLSSPVTKSFLRGECGG
PAGGGGGTHTCPPCPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVCTLPPSRD

ELTKNQVSLSCAVKGFYPSDIAVEWESNGQPENNYKTTPPVLDSDGSFFLVSKLTVDKSRWQQGNVFSCSV

| | |
|---|---|
| MHEALHNHYTQKSLSLSPGKGGGGSGQRVAAHITGTRGRSNTLSSPNSKNEKALGRKINSWESSRSGHSFL | |
| SNLHLRNGELVIHEKGFYYIYSQTYFRFQEEIKENTKNDKQMVQYIYKYTSYPDPILLMKSARNSCWSKDA | |
| EYGLYSIYQGGIFELKENDRIFVSVTNEHLIDMDHEASFFGAFLV | |
| L: 17v29: h3Vk-CL-link-hinge-G1Fc-linker-NKG2D domain | 67 |
| QAVVTQEPSLTVSPGGTVTLTCGSSTGAVTTSNYANWVQEKPGQAFRGLIGGTNKRAPGTPARFSGSLLGG | |
| KAALTLSGAQPEDEAEYYCALWYSNLWVFGCGTKLTVLRTVAAPSVFIFPPSDEQLKSGTASVVCLLNNFY | |
| PREAKVQWKVDNALQSGNSQESVTEQDSKDSTYSLSSTLTLSKADYEKHKVYACEVTHQGLSSPVTKSFN | |
| RGECGGPAGGGGGTHTCPPCPAPEAAGGPSVFLFPPKPKDTLMISRTPEVTCVVVDVSHEDPEVKFNWYVD | |
| GVEVHNAKTKPREEQYNSTYRVVSVLTVLHQDWLNGKEYKCKVSNKALPAPIEKTISKAKGQPREPQVCT | |

LPPSRDELTKNQVSLSCAVKGFYPSDIAVEWESNGQPENNYKTTPPVLDSDGSFFLVSKLTVDKSRWQQGN
VFSCSVMHEALHNHYTQKSLSLSPGKGAPGGGGSAVFLNSLFNQEVQIPLTESYCGPCPKNWICYKNNCYQ
FFDESKNWYESQASCMSQNASLLKVYSKEDQDLLKLVKSYHWMGLVHIPTNGSWQWEDGSILSPNLLTIIE

| | |
|---|---|
| MQKGDCALYASSFKGYIENCSTPNTYICMQRTV | |
| H: 18v42: h3Vh-MCH1wt-link-hinge-G1Fc-linker-NKG2D domain | 68 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGSASAPTLFPLVS

CENSPSDTSSVAVGCLAQDFLPDSITFSWKYKNNSDISSTRGFPSVLRGGKYAATSQVLLPSKDVMQGTDE
HVVCKVQHPNGNKEKNVPLPAGGGGGTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIEKTI

SKAKGQPREPQVYTLPPCRDELTKNQVSLWCLVKGFYPSDIAVEWESNGQPENNYKTTPPVLDSDGSFFLY
SKLTVDKSRWQQGNVFSCSVMHEALHNHYTQKSLSLSPGKGAPGGGGSAVFLNSLFNQEVQIPLTESYCG

| | |
|---|---|
| PCPKNWICYKNNCYQFFDESKNWYESQASCMSQNASLLKVYSKEDQDLLKLVKSYHWMGLVHIPTNGSW | |
| QWEDGSILSPNLLTIIEMQKGDCALYASSFKGYIENCSTPNTYICMQRTV | |
| L: 13: NKG2D-linker-hinge-G1Fc | 69 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVPAGGGGGTHTCP | |
| PCPAPEAAGGPSVFLFPPKPKDTLMISRTPEVTCVVVDVSHEDPEVKFNWYVDGVEVHNAKTKPREEQYNS | |
| TYRVVSVLTVLHQDWLNGKEYKCKVSNKALPAPIEKTISKAKGQPREPQVCTLPPSRDELTKNQVSLSCAV | |

KGFYPSDIAVEWESNGQPENNYKTTPPVLDSDGSFFLVSKLTVDKSRWQQGNVFSCSVMHEALHNHYTQK

| | |
|---|---|
| SLSLSPGK | |
| 14: h3scFv-NKG2D-linker-hinge-G1Fc | 70 |
| EVQLVESGGGLVQPGGSLKLSCAASGFTFNTYAMNWVRQAPGKGLEWVGRIRSKYNNYATYYADSVKG | |

RFTISRDDSKNTLYLQMNSLKTEDTAVYYCVRHGNFGNSYVSWFAYWGQGTLVTVSSGGGGSGGGGSGG
GGSQAVVTQEPSLTVSPGGTVTLTCGSSTGAVTTSNYANWVQQKPGQAFRGLIGGTNKRAPGTPARFSGSL

LGGKAALTLSGAQPEDEAEYYCALWYSNLWVFGGGTKLTVLASGGGGGPLTESYCGPCPKNWICYKNNC

YQFFDESKNWYESQASCMSQNASLLKVYSKEDQDLLKLVKSYHWMGLVHIPTNGSWQWEDGSILSPNLL

TIIEMQKGDCALYASSFKGYIENCSTPNTYICMQRTVPAGGGGGTHTCPPCPAPEAAGGPSVFLFPPKPKDT
LMISRTPEVTCVVVDVSHEDPEVKFNWYVDGVEVHNAKTKPREEQYNSTYRVVSVLTVLHQDWLNGKEY

KCKVSNKALPAPIEKTISKAKGQPREPQVYTLPPCRDELTKNQVSLWCLVKGFYPSDIAVEWESNGQPENN

| | |
|---|---|
| YKTTPPVLDSDGSFFLYSKLTVDKSRWQQGNVFSCSVMHEALHNHYTQKSLSLSPGK | |
| NKG2D-linker-G4Fc-linker-b2GP1 | 71 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGAPGGGGGPPCP | |
| PCPAPEAAGASSVFLFPPKPKDTLMISRTPEVTCVVVDVSQEDPEVQFNWYVDGVEVHNAKTKPREEQFNS | |
| TYRVVSVLTVLHQDWLNGKEYKCKVSNKGLPSSIEKTISKAKGQPREPQVYTLPPSQEEMTKNQVSLTCLV | |

KGFYPSDIAVEWESNGQPENNYKTTPPVLDSDGSFFLYSRLTVDKSRWQEGNVFSCSVMHEALHNHYTQK

| | |
|---|---|
| SLSLSPGKGGGGGGASCKVPVKKATVVYQGERVKIQEKFKNGMLHGDKVSFFCKNKEKKCSYTEDAQCI | |
| DGTIEVPKCFKEHSSLAFWKTDASDVKPC | |
| NKG2D-linker-G4Fc-linker-CD137L | 72 |
| QIPLTESYCGPCPKNWICYKNNCYQFFDESKNWYESQASCMSQNASLLKVYSKEDQDLLKLVKSYHWMG | |
| LVHIPTNGSWQWEDGSILSPNLLTIIEMQKGDCALYASSFKGYIENCSTPNTYICMQRTVGAPGGGGGPPCP | |
| PCPAPEAAGASSVFLFPPKPKDTLMISRTPEVTCVVVDVSQEDPEVQFNWYVDGVEVHNAKTKPREEQFNS | |
| TYRVVSVLTVLHQDWLNGKEYKCKVSNKGLPSSIEKTISKAKGQPREPQVYTLPPSQEEMTKNQVSLTCLV | |

KGFYPSDIAVEWESNGQPENNYKTTPPVLDSDGSFFLYSRLTVDKSRWQEGNVFSCSVMHEALHNHYTQK

| | |
|---|---|
| SLSLSPGKGGGGGGMFAQLVAQNVLLIDGPLSWYSDPGLAGVSLTGGLSYKEDTKELVVAKAGVYYVFF | |
| QLELRRVVAGEGSGSVSLALHLQPLRSAAGAAALALTVDLPPASSEARNSAFGFQGRLLHLSAGQRLGVHL | |
| HTEARARHAWQLTQGATVLGLFRVTP | |
| CD137L-linker-G4Fc-linker-b2GP1 | 73 |
| QGMFAQLVAQNVLLIDGPLSWYSDPGLAGVSLTGGLSYKEDTKELVVAKAGVYYVFFQLELRRVVAGEG | |
| SGSVSLALHLQPLRSAAGAAALALTVDLPPASSEARNSAFGFQGRLLHLSAGQRLGVHLHTEARARHAWQ | |
| LTQGATVLGLFRVTPAGGGGSGGPPCPPCPAPEAAGASSVFLFPPKPKDTLMISRTPEVTCVVVDVSQEDPE | |
| VQFNWYVDGVEVHNAKTKPREEQFNSTYRVVSVLTVLHQDWLNGKEYKCKVSNKGLPSSIEKTISKAKG | |
| QPREPQVYTLPPSQEEMTKNQVSLTCLVKGFYPSDIAVEWESNGQPENNYKTTPPVLDSDGSFFLYSRLTV | |
| DKSRWQEGNVFSCSVMHEALHNHYTQKSLSLSPGKGGGGGGASCKVPVKKATVVYQGERVKIQEKFKNG | |
| MLHGDKVSFFCKNKEKKCSYTEDAQCIDGTIEVPKCFKEHSSLAFWKTDASDVKPC | |
| TRAIL-linker-G4Fc-linker-b2GP1 | 74 |

QRVAAHITGTRGRSNTLSSPNSKNEKALGRKINSWESSRSGHSFLSNLHLRNGELVIHEKGFYYIYSQTYFRF
QEEIKENTKNDKQMVQYIYKYTSYPDPILLMKSARNSCWSKDAEYGLYSIYQGGIFELKENDRIFVSVTNE
HLIDMDHEASFFGAFLVGGGGSGGRVAAHITGTRGRSNTLSSPNSKNEKALGRKINSWESSRSGHSFLSNLH
LRNGELVIHEKGFYYIYSQTYFRFQEEIKENTKNDKQMVQYIYKYTSYPDPILLMKSARNSCWSKDAEYGL
YSIYQGGIFELKENDRIFVSVTNEHLIDMDHEASFFGAFLVGGGGSGGRVAAHITGTRGRSNTLSSPNSKNE
KALGRKINSWESSRSGHSFLSNLHLRNGELVIHEKGFYYIYSQTYFRFQEEIKENTKNDKQMVQYIYKYTSY
PDPILLMKSARNSCWSKDAEYGLYSIYQGGIFELKENDRIFVSVTNEHLIDMDHEASFFGAFLVGAPGGGGG
PPCPPCPAPEAAGASSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSVMHEALHNH
YTQKSLSLSPGKGGGGGGASCKVPVKKATVVYQGERVKIQEKFKNGMLHGDKVSFFCKNKEKKCSYTED
AQCIDGTIEVPKCFKEHSSLAFWKTDASDVKPC

| | |
|---|---|
| L Chain: h133Vk-CL | 75 |
| EIVLTQSPATLSLSPGERATLSCRASQSIGTSIHWYQQKPGQAPRLLIKYASESISGIPARFSGSGSGTDFTLTIS | |
| SLEPEDFAVYYCQQSYIWPTTFGQGTKLEIKRTVAAPSVFIFPPSDEQLKSGTASVVCLLNNFYPREAKVQW | |
| KVDNALQSGNSQESVTEQDSKDSTYSLSSTLTLSKADYEKHKVYACEVTHQGLSSPVTKSFNRGEC | |
| H Chain: h133Vh-CH1-G4Fc-linker-CD137L domain | 76 |
| QVQLVQSGAEVKKPGASVKVSCKASGYTFTNYWLGWVRQAPGQGLEWMGNIFPGGDYTNYNENFKGRV | |

TMTRDTSISTAYMELSRLRSDDTAVYYCARSMDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKK

VESKYGPPCPPCPAPEAAGASSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQEGNVFSCSVMH
EALHNHYTQKSLSLSLPKGGGGGGMFAQLVAQNVLLIDGPLSWYSDPGLAGVSLTGGLSYKEDTKELVVA

KAGVYYVFFQLELRRVVAGEGSGSVSLALHLQPLRSAAGAAALALTVDLPPASSEARNSAFGFQGRLLHLS

| | |
|---|---|
| AGQRLGVHLHTEARARHAWQLTQGATVLGLFRVTP | |
| H Chains: h133 Vh-CH1-Ig hinge-CH2—CH3(hole)-linker-TRAIL-linker- | 77 |
| TRAIL | |
| QVQLVQSGAEVKKPGASVKVSCKASGYTFTNYWLGWVRQAPGQGLEWMGNIFPGGDYTNYNENFKGRV | |

TMTRDTSISTAYMELSRLRSDDTAVYYCARSMDYWGQGTLVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKR

VEPKSCDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVCTLPPSRD

ELTKNQVSLSCAVKGFYPSDIAVEWESNGQPENNYKTTPPVLDSDGSFFLVSKLTVDKSRWQQGNVFSCSV
MHEALHNHYTQKSLSLSPGKGGGGSGQRVAAHITGTRGRSNTLSSPNSKNEKALGRKINSWESSRSGHSFL
SNLHLRNGELVIHEKGFYYIYSQTYFRFQEEIKENTKNDKQMVQYIYKYTSYPDPILLMKSARNSCWSKDA
EYGLYSIYQGGIFELKENDRIFVSVTNEHLIDMDHEASFFGAFLVGGGGSGGQRVAAHITGTRGRSNTLSSP
NSKNEKALGRKINSWESSRSGHSFLSNLHLRNGELVIHEKGFYYIYSQTYFRFQEEIKENTKNDKQMVQYIY

| | |
|---|---|
| KYTSYPDPILLMKSARNSCWSKDAEYGLYSIYQGGIFELKENDRIFVSVTNEHLIDMDHEASFFGAFLV | |
| H Chains: h133Vh-CH1-Ig hinge-CH2—CH3(knob)-linker-TRAIL | 78 |
| QVQLVQSGAEVKKPGASVKVSCKASGYTFTNYWLGWVRQAPGQGLEWMGNIFPGGDYTNYNENFKGRV | |

TMTRDTSISTAYMELSRLRSDDTAVYYCARSMDYWGQGTLVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKR

VEPKSCDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPPCR
DELTKNQVSLWCLVKGFYPSDIAVEWESNGQPENNYKTTPPVLDSDGSFFLYSKLTVDKSRWQQGNVFSC
SVMHEALHNHYTQKSLSLSPGKGGGGSGQRVAAHITGTRGRSNTLSSPNSKNEKALGRKINSWESSRSGHS
FLSNLHLRNGELVIHEKGFYYIYSQTYFRFQEEIKENTKNDKQMVQYIYKYTSYPDPILLMKSARNSCWSK

DAEYGLYSIYQGGIFELKENDRIFVSVTNEHLIDMDHEASFFGAFLVGGGGSGGQRVAAHITGTRGRSNTLS
SPNSKNEKALGRKINSWESSRSGHSFLSNLHLRNGELVIHEKGFYYIYSQTYFRFQEEIKENTKNDKQMVQY

| | |
|---|---|
| IYKYTSYPDPILLMKSARNSCWSKDAEYGLYSIYQGGIFELKENDRIFVSVTNEHLIDMDHEASFFGAFLV | |
| CD40L-linker-CD40L-linker-CD40L-linker-tIg hinge-G4Fc- | 79 |
| linker-b2GP1 | |
| QIAAHVISEASSKTTSVLQWAEKGYYTMSNNLVTLENGKQLTVKRQGLYYIYAQVTFCSNREASSQAPFIA | |
| SLCLKSPGRFERILLRAANTHSSAKPCGQQSIHLGGVFELQPGASVFVNVTDPSQVSHGTGFTSFGLLKLGG | |
| GGSGGIAAHVISEASSKTTSVLQWAEKGYYTMSNNLVTLENGKQLTVKRQGLYYIYAQVTFCSNREASSQ | |
| APFIASLCLKSPGRFERILLRAANTHSSAKPCGQQSIHLGGVFELQPGASVFVNVTDPSQVSHGTGFTSFGLL | |

KLGGGGSGGIAAHVISEASSKTTSVLQWAEKGYYTMSNNLVTLENGKQLTVKRQGLYYIYAQVTFCSNRE

| | |
|---|---|
| ASSQAPFIASLCLKSPGRFERILLRAANTHSSAKPCGQQSIHLGGVFELQPGASVFVNVTDPSQVSHGTGFTS | |
| FGLLKLGGAPAGGGGGGPPCPPCPAPEAAGASSVFLFPPKPKDTLMISRTPEVTCVVVDVSQEDPEVQFNW | |
| YVDGVEVHNAKTKPREEQFNSTYRVVSVLTVLHQDWLNGKEYKCKVSNKGLPSSIEKTISKAKGQPREPQ | |
| VYTLPPSQEEMTKNQVSLTCLVKGFYPSDIAVEWESNGQPENNYKTTPPVLDSDGSFFLYSRLTVDKSRWQ | |
| EGNVFSCSVMHEALHNHYTQKSLSLSLGKGGGGGGASCKVPVKKATVVYQGERVKIQEKFKNGMLHGD | |
| KVSFFCKNKEKKCSYTEDAQCIDGTIEVPKCFKEHSSLAFWKTDASDVKPC | |
| L: hTVk-CL-truncated Ig hinge-CD137L domain | 80 |
| DIQLTQSPSSLSASVGDRVSITCKASQDVSIAVAWYQQKPGKAPKLLIYSASYRTTGVPDRFSGSGSGTDFTL | |

TISSLQPEDFAVYYCQQHYITPLTFGAGTKVEIKRTVAAPSVFIFPPSDEQLKSGTASVVCLLNNFYPREAKV

| | |
|---|---|
| QWKVDNALQSGNSQESVTEQDSKDSTYSLSSTLTLSKADYEKHKVYACEVTHQGLSSPVTKSFNRGECGG | |
| GTHTCPPCPAPEAAGGPGGGMFAQLVAQNVLLIDGPLSWYSDPGLAGVSLTGGLSYKEDTKELVVAKAGV | |
| YYVFFQLELRRVVAGEGSGSVSLALHLQPLRSAAGAAALALTVDLPPASSEARNSAFGFQGRLLHLSAGQR | |
| LGVHLHTEARARHAWQLTQGATVLGLFRVTP | |
| H: hTVh-CH1-truncated Ig hinge-CD137L domain | 81 |
| QVQLGQSGSELKKPGASVKVSCKASGYTFTNYGMNWVKQAPGQGLKWMGWINTYTGEPTYTDDFKGRF | |

AFSLDTSVSTAYLQISSLKADDTAVYFCARGGFGSSYWYFDVWGQGSLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS

| | |
|---|---|
| NTKVDKKVGGGTHTCPPCPAPEAAGGPGGGMFAQLVAQNVLLIDGPLSWYSDPGLAGVSLTGGLSYKED | |
| TKELVVAKAGVYYVFFQLELRRVVAGEGSGSVSLALHLQPLRSAAGAAALALTVDLPPASSEARNSAFGF | |
| QGRLLHLSAGQRLGVHLHTEARARHAWQLTQGATVLGLFRVTP | |
| L: h133 Vk-truncated Ig hinge-CD137L domain | 82 |
| EIVLTQSPATLSLSPGERATLSCRASQSIGTSIHWYQQKPGQAPRLLIKYASESISGIPARFSGSGSGTDFTLTIS | |
| SLEPEDFAVYYCQQSYIWPTTFGQGTKLEIKGGGTHTCPPCPAPEAAGGPGGGMFAQLVAQNVLLIDGPLS | |
| WYSDPGLAGVSLTGGLSYKEDTKELVVAKAGVYYVFFQLELRRVVAGEGSGSVSLALHLQPLRSAAGAA | |
| ALALTVDLPPASSEARNSAFGFQGRLLHLSAGQRLGVHLHTEARARHAWQLTQGATVLGLFRVTP | |
| H: h133Vh-truncated Ig hinge-CD137L domain | 83 |
| QVQLVQSGAEVKKPGASVKVSCKASGYTFTNYWLGWVRQAPGQGLEWMGNIFPGGDYTNYNENFKGRV | |
| TMTRDTSISTAYMELSRLRSDDTAVYYCARSMDYWGQGTLVTVSSGGGTHTCPPCPAPEAAGGPGGGMF | |
| AQLVAQNVLLIDGPLSWYSDPGLAGVSLTGGLSYKEDTKELVVAKAGVYYVFFQLELRRVVAGEGSGSVS | |
| LALHLQPLRSAAGAAALALTVDLPPASSEARNSAFGFQGRLLHLSAGQRLGVHLHTEARARHAWQLTQGA | |
| TVLGLFRVTP | |
| h133Vh-Vk (scFv)-linker-CD137L domain-linker-CD137L domain | 84 |
| QVQLVQSGAEVKKPGASVKVSCKASGYTFTNYWLGWVRQAPGQGLEWMGNIFPGGDYTNYNENFKGRV | |
| TMTRDTSISTAYMELSRLRSDDTAVYYCARSMDYWGQGTLVTVSSGGGGSGGGGSGGGGSEIVLTQSPAT | |

LSLSPGERATLSCRASQSIGTSIHWYQQKPGQAPRLLIKYASESISGIPARFSGSGSGTDFTLTISSLEPEDFAV

| | |
|---|---|
| YYCQQSYIWPTTFGQGTKLEIKGGGGASGMFAQLVAQNVLLIDGPLSWYSDPGLAGVSLTGGLSYKEDTK | |
| ELVVAKAGVYYVFFQLELRRVVAGEGSGSVSLALHLQPLRSAAGAAALALTVDLPPASSEARNSAFGFQG | |
| RLLHLSAGQRLGVHLHTEARARHAWQLTQGATVLGLFRVTPGGGGSGGGMFAQLVAQNVLLIDGPLSWY | |
| SDPGLAGVSLTGGLSYKEDTKELVVAKAGVYYVFFQLELRRVVAGEGSGSVSLALHLQPLRSAAGAAALA | |
| LTVDLPPASSEARNSAFGFQGRLLHLSAGQRLGVHLHTEARARHAWQLTQGATVLGLFRVTP | |
| h133scFv -linker-CD137L-linker-CD137L-linker-b2GP1 | 85 |
| QVQLVQSGAEVKKPGASVKVSCKASGYTFTNYWLGWVRQAPGQGLEWMGNIFPGGDYTNYNENFKGRV | |

TMTRDTSISTAYMELSRLRSDDTAVYYCARSMDYWGQGTLVTVSSGGGGSGGGGSGGGGSEIVLTQSPAT
LSLSPGERATLSCRASQSIGTSIHWYQQKPGQAPRLLIKYASESISGIPARFSGSGSGTDFTLTISSLEPEDFAV

| | |
|---|---|
| YYCQQSYIWPTTFGQGTKLEIKGGGGASGMFAQLVAQNVLLIDGPLSWYSDPGLAGVSLTGGLSYKEDTK | |
| ELVVAKAGVYYVFFQLELRRVVAGEGSGSVSLALHLQPLRSAAGAAALALTVDLPPASSEARNSAFGFQG | |
| RLLHLSAGQRLGVHLHTEARARHAWQLTQGATVLGLFRVTPGGGGSGGGMFAQLVAQNVLLIDGPLSWY | |
| SDPGLAGVSLTGGLSYKEDTKELVVAKAGVYYVFFQLELRRVVAGEGSGSVSLALHLQPLRSAAGAAALA | |
| LTVDLPPASSEARNSAFGFQGRLLHLSAGQRLGVHLHTEARARHAWQLTQGATVLGLFRVTPGGGGGGAS | |
| CKVPVKKATVVYQGERVKIQEKFKNGMLHGDKVSFFCKNKEKKCSYTEDAQCIDGTIEVPKCFKEHSSLA | |
| FWKTDASDVKPC | |
| Tim4-linker-CD137L-linker-CD137L | 86 |
| ETVVTEVLGHRVTLPCLYSSWSHNSNSMCWGKDQCPYSGCKEALIRTDGMRVTSRKSAKYRLQGTIPRGD | |
| VSLTILNPSESDSGVYCCRIEVPGWFNDVKINVRLNLQGGGGGSGGMFAQLVAQNVLLIDGPLSWYSDPGL | |
| AGVSLTGGLSYKEDTKELVVAKAGVYYVFFQLELRRVVAGEGSGSVSLALHLQPLRSAAGAAALALTVDL | |
| PPASSEARNSAFGFQGRLLHLSAGQRLGVHLHTEARARHAWQLTQGATVLGLFRVTPGGGGSGGGMFAQ | |
| LVAQNVLLIDGPLSWYSDPGLAGVSLTGGLSYKEDTKELVVAKAGVYYVFFQLELRRVVAGEGSGSVSLA | |
| LHLQPLRSAAGAAALALTVDLPPASSEARNSAFGFQGRLLHLSAGQRLGVHLHTEARARHAWQLTQGATV | |
| LGLFRVTP | |
| TRAIL-linker-TRAIL-linker-TRAIL-linker-b2GP1-linker b2GP1 | 87 |

QRVAAHITGTRGRSNTLSSPNSKNEKALGRKINSWESSRSGHSFLSNLHLRNGEL VIHEKGFYYIYSQTYFRF
QEEIKENTKNDKQMVQYIYKYTSYPDPILLMKSARNSCWSKDAEYGLYSIYQGGIFELKENDRIFVSVTNE
HLIDMDHEASFFGAFLVGGGGSGGQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYPDPILLMKSARNSCWSKDAEYG
LYSIYQGGIFELKENDRIFVSVTNEHLIDMDHEASFFGAFLVGGGGSGGQRVAAHITGTRGRSNTLSSPNSK
NEKALGRKINSWESSRSGHSFLSNLHLRNGELVIHEKGFYYIYSQTYFRFQEEIKENTKNDKQMVQYIYKYT
SYPDPILLMKSARNSCWSKDAEYGLYSIYQGGIFELKENDRIFVSVTNEHLIDMDHEASFFGAFLVGGGGGG
ASCKVPVKKATVVYQGERVKIQEKFKNGMLHGDKVSFFCKNKEKKCSYTEDAQCIDGTIEVPKCFKEHSS
LAFWKTDASDVKPCGGGGGGASCKVPVKKATVVYQGERVKIQEKFKNGMLHGDKVSFFCKNKEKKCSY
TEDAQCIDGTIEVPKCFKEHSSLAFWKTDASDVKPC

What is claimed is:

1. The antibody-derived recombinant antigen binding molecule of claim 4 further comprising a scaffold with at least a first subunit having an N-terminus and a C-terminus, at least a second subunit having an N-terminus and a C-terminus, at least a third subunit having an N-terminus and a C-terminus, and at least a fourth subunit having an N-terminus and a C-terminus; and
the first subunit, comprising from the N-terminus to the C-terminus, a first antigen binding domain (A) and a CL light chain constant region of an Igκ or an Igλ;
the second subunit, comprising from the N-terminus to the C-terminus, a second antigen binding domain (B), a CH1 heavy chain constant region, an immunoglobulin hinge region, a heavy chain Fc domain from an IgG, and a third antigen binding domain (C);
the third subunit, comprising from the N-terminus to the C-terminus, a fourth antigen binding domain (D), a CL light chain constant region of an Igκ or an Igλ, an immunoglobulin hinge region, a heavy chain Fc domain from an IgG, and a fifth antigen binding domain (E); and
the fourth subunit, comprising from the N-terminus to the C-terminus, a sixth antigen binding domain (F) and a CH1 heavy chain constant region;
wherein high-affinity binding between the CL light chain constant region in the first subunit and the CH1 heavy chain constant region in the second subunit and a second high-affinity binding between the CL light chain constant region in the third subunit and the CH1 heavy chain constant region in the fourth subunit drive heterodimerization of the molecule.

2. The antibody-derived recombinant antigen binding molecule of claim 1, wherein the scaffold comprises a first subunit having an N-terminus and a C-terminus, a second subunit having an N-terminus and a C-terminus, and a third subunit having an N-terminus and a C-terminus; wherein
the first subunit, comprising from the N-terminus to the C-terminus, a first antigen binding domain (A) and a CL light chain constant region;
the second subunit, comprising from the N-terminus to the C-terminus, a second antigen binding domain (B), a CH1 heavy chain constant region, an immunoglobulin hinge region, a heavy chain Fc domain from an IgG, and a third antigen binding domain (C); and
the third subunit, comprising from the N-terminus to the C-terminus, a fourth antigen binding domain (D), an immunoglobulin hinge region, a heavy chain Fc domain from an IgG, and a fifth antigen binding domain (E);
wherein high-affinity binding between the CL light chain constant region in the first subunit and the CH1 heavy chain constant region in the second subunit drive heterodimerization of the molecule.

3. The antibody-derived recombinant antigen binding molecule of claim 1, wherein the scaffold comprises a first subunit having an N-terminus and a C-terminus and a second subunit having an N-terminus and a C-terminus; wherein
the first subunit, comprising from the N-terminus to the C-terminus, a first antigen binding domain (A), a CL light chain constant region, an immunoglobulin hinge region, a heavy chain Fc domain from an IgG, and a second antigen binding domain (B);
the second subunit, comprising from the N-terminus to the C-terminus, a third antigen binding domain (C), a CH1 heavy chain constant region, an immunoglobulin hinge region, a heavy chain Fc domain from an IgG, and a fourth antigen binding domain (D); and
wherein high-affinity binding between the CL light chain constant region in the first subunit and the CH1 heavy chain constant region in the second subunit drive heterodimerization of the molecule.

4. An antibody-derived recombinant antigen binding molecule comprising a scaffold with a first subunit having an N-terminus and a C-terminus and a second subunit having an N-terminus and a C-terminus; and
the first subunit, comprising from the N-terminus to the C-terminus, a first antigen binding domain (A) and a CH1 heavy chain constant region; and
the second subunit, comprising from the N-terminus to the C-terminus, a second antigen binding domain (B) and a CL light chain constant region of an Igκ or an Igλ;
wherein the first subunit and the second subunit are linked to a chimeric antigen receptor, wherein the chimeric antigen receptor comprises a hinge region, a transmembrane domain, and at least one intracellular signaling domain; and
wherein the high-affinity binding between the CH1 heavy chain constant region in the first subunit and the CL light chain constant region in the first subunit drive heterodimerization of the molecule.

5. The antibody-derived recombinant antigen binding molecule of claim 4, wherein the chimeric antigen receptor is a phagocytic chimeric antigen receptor.

6. The antibody-derived recombinant antigen binding molecule of claim 4, wherein the CH1 heavy chain constant region is selected from the group of an IgM, an IgA, an IgG₂, or an IgG₃.

7. The antibody-derived recombinant antigen binding molecule of claim 5, wherein the antigen binding domain is a Fv, a F(ab′)₂, a Fab, a scFv, a VHH based single antigen binding domain, or natural protein domains.

8. The antibody-derived recombinant antigen binding molecule of claim 7, wherein the natural protein domains comprise (i) the immune checkpoint protein PD1, (ii) the cytokine IL15 or IL 15-alpha, or (iii) the natural killer receptor NKG2D, NKp44, NKp46, NKp30, or NKRP1A.

9. (canceled)

10. (canceled)

11. (canceled)

12. The antibody-derived recombinant antigen binding molecule of claim 5, wherein the N-terminus of at least one antigen binding domain is linked to an antigen binding molecule, wherein an immunoglobulin hinge region links the N-terminus of the antigen binding domain to the antigen binding molecule.

13. The antibody-derived recombinant antigen binding molecule of claim 12, wherein the antigen binding molecule is an IL15 alpha receptor, IL15 receptor, or NKG2D dimer.

14. (canceled)

15. (canceled)

16. The antibody-derived recombinant antigen binding molecule of claim 1, wherein the Fc domain comprises either a disulfide bridge or a knob-into-hole modification, or the Fc domain comprises both a disulfide bridge and a knob-into-hole modification.

17. The antibody-derived recombinant antigen binding molecule of claim 1, wherein the Fc domain comprises a CH2 domain of an IgG and one or more CH3 domain of an IgG.

18. (canceled)

19. The antibody-derived recombinant antigen binding molecule of claim 17, wherein the C-terminus of at least one CH3 domain is linked to an antigen binding molecule.

20. The antibody-derived recombinant antigen binding molecule of claim 17, wherein the antigen binding molecule is PD-1.

21. The antibody-derived recombinant antigen binding molecule of claim 1, wherein at least one antigen binding domain is specific for binding to a tumor associated antigen, a tumor specific antigen, a leukocyte antigen, an effector cell receptor, a cytokine receptor, a Toll-like receptor (TLR), a phagocytic receptor, or an immune checkpoint protein.

22. The antibody-derived recombinant antigen binding molecule of claim 21, wherein the tumor associated antigen is Trop2, Her2, CD19, CD22, MR1, CD1, MICA/B, ULBP, CLEC2D, or NKp44L.

23. The antibody-derived recombinant antigen binding molecule of claim 21, wherein the effector cell receptor is TCRs, NK receptors, DR5, phagocytic receptors, or TLRs.

24. The antibody-derived recombinant antigen binding molecule of claim 21, wherein the cytokine receptor is IL15, IL12, IL7, PD-1, PD-L1, TIGIT, or CTLA-4.

25. (canceled)

26. The antibody-derived recombinant antigen binding molecule of claim 1, wherein the heterodimerization is further stabilized by (i) modifying the interaction between the CL light chain constant region and the CH1 heavy chain constant region, (ii) the addition of a disulfide bridge to the Fc domain, (iii) the addition of a knob-into-hole modification.

27. (canceled)

28. (canceled)

29. The antibody-derived recombinant antigen binding molecule of claim 1, wherein the immunoglobulin hinge region (i) is 3-9 amino acids in length, (ii) comprises glycine amino acids, and/or (iii) comprises proline amino acids.

30. (canceled)

31. (canceled)

32. The antibody-derived recombinant antigen binding molecule of claim 4, wherein the transmembrane domain is from a CD 28 T cell.

33. (canceled)

34. The antibody-derived recombinant antigen binding molecule of claim 4, wherein the intracellular signaling domain is selected from the group consisting of CD137, CD3ζ, CD40, ICOS, CD2, DAP10, DAP12, SIRPa, CR1, CD11b+CD18, aV+b3/b5, MER tyrosine kinase, AXL tyrosine kinase, and scavenger Rcs.

35. (canceled)

37. The antibody-derived recombinant antigen binding molecule of claim 4 further comprising a scaffold with at least a first subunit having an N-terminus and a C-terminus, at least a second subunit having an N-terminus and a C-terminus; and at least one subunit of a tumor necrosis factor superfamily (TNFSF) ligand;
the first subunit, comprising from the N-terminus to the C-terminus, a first antigen binding domain, a CH1 heavy chain constant region, an immunoglobulin hinge region, a heavy chain Fc domain from an IgG, and a second antigen binding domain; and
the second subunit, comprising from the N-terminus to the C-terminus, a third antigen binding domain, a CL light chain constant region of an Igκ or an Igλ, an immunoglobulin hinge region, a heavy chain Fc domain from an IgG, and a fourth antigen binding domain; and
wherein the TNFSF ligand is linked to a terminal end of the first and/or second subunit.

38. The antibody-derived recombinant antigen binding molecule of claim 37, comprising one or more subunit of the TNFSF ligand.

39. (canceled)

40. The antibody-derived recombinant antigen binding molecule of claim 37 comprising an antigen binding domain selected from the group of Fab, Fv, scFv, scFv and a receptor, a receptor, and two or more receptors.
